SEQUENCE LISTING (35 sequences)

SEQ ID NO: 1 - DNA, 70 bp, Artificial Sequence
  Description of Artificial Sequence Synthetic oligonucleotide
gcaaatgggcggtaggcgtgtacggtgggaggtttatataagcagagctcgtttagtgaaccgtcagatc

SEQ ID NO: 2 - DNA, 380 bp, Artificial Sequence
  Description of Artificial Sequence Synthetic polynucleotide
cattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 3 - DNA, 559 bp, Artificial Sequence
  Description of Artificial Sequence Synthetic polynucleotide
cgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccctt

SEQ ID NO: 4 - DNA, 511 bp, Artificial Sequence
  Description of Artificial Sequence Synthetic polynucleotide
taccgggtaggggaggcgcttttcccaaggcagtctggagcatgcgctttagcagccccgctggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccatcta

SEQ ID NO: 5 - DNA, 153 bp, Artificial Sequence
  Description of Artificial Sequence Synthetic polynucleotide
aactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatc

SEQ ID NO: 6 - DNA, 546 bp, Artificial Sequence
  Description of Artificial Sequence Synthetic polynucleotide
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 7 - DNA, 2559 bp, Homo sapiens
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 8 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 9 - DNA, 2628 bp, Homo sapiens
atggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 10 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 11 - DNA, 2619 bp, Homo sapiens
atggccacagcttgtaaaagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 12 - protein, 872 aa, Homo sapiens
MATACKRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 13 - DNA, 2619 bp, Artificial Sequence
  Description of Artificial Sequence Synthetic polynucleotide
atgtacaggatgcaactcctgtcttgcattgcactaagtcttgcacttgtcacaaacagtatgaagcgagcagctgcaaagcatctaatagaacgctactaccaccagttaactgagggctgtggaaatgaagcctgcacgaatgagttttgtgcttcctgtccaacttttcttcgtatggataataatacagcagctattaaagccctcgagctttataagattaatgcaaaactctgtgatcctcatccctccaagaaaggagcaagctcagcttaccttgagaactcgacaggtgcccccaacaacacctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatacgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtacaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgataactctacaaaattgttttggtttaatccatcttcttttgaaactgagggtcagtttactctgattggcatagtactgggtctggctatttacaataacactatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagaataatacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 14 - protein, 872 aa, Artificial Sequence
  Description of Artificial Sequence Synthetic polypeptide
MYRMQLLSCIALSLALVTNSMKRAAAKHLIERYYHQLTEGCGNEACTNEFCASCPTFLRMDNNTAAIKALELYKINAKLCDPHPSKKGASSAYLENSTGAPNNTCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNTQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSTNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDNSTKLFWFNPSSFETEGQFTLIGIVLGLAIYNNTILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQNNTGTDRAPVGGLGKLKMIIAKNGPDTERLPTSHTCFNVLLLPEYSSKEKLKERLLKAITYAKGFGML

SEQ ID NO: 15 - DNA, 2688 bp, Artificial Sequence
  Description of Artificial Sequence Synthetic polynucleotide
atgtacaggatgcaactcctgtcttgcattgcactaagtcttgcacttgtcacaaacagtatggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatacagcagctattaaagccctcgagctttataagattaatgcaaaactctgtgatcctcatccctccaagaaaggagcaagctcagcttaccttgagaactcgacaggtgcccccaacaacacctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatacgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtacaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgataactctacaaaattgttttggtttaatccatcttcttttgaaactgagggtcagtttactctgattggcatagtactgggtctggctatttacaataacactatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagaataatacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 16 - protein, 895 aa, Artificial Sequence
  Description of Artificial Sequence Synthetic polypeptide
MYRMQLLSCIALSLALVTNSMEKLHQCYWKSGEPQSDDIEASRMKRAAAKHLIERYYHQLTEGCGNEACTNEFCASCPTFLRMDNNTAAIKALELYKINAKLCDPHPSKKGASSAYLENSTGAPNNTCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNTQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSTNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDNSTKLFWFNPSSFETEGQFTLIGIVLGLAIYNNTILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQNNTGTDRAPVGGLGKLKMIIAKNGPDTERLPTSHTCFNVLLLPEYSSKEKLKERLLKAITYAKGFGML

SEQ ID NO: 17 - DNA, 2679 bp, Artificial Sequence
  Description of Artificial Sequence Synthetic polynucleotide
atgtacaggatgcaactcctgtcttgcattgcactaagtcttgcacttgtcacaaacagtatggccacagcttgtaaaagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatacagcagctattaaagccctcgagctttataagattaatgcaaaactctgtgatcctcatccctccaagaaaggagcaagctcagcttaccttgagaactcgacaggtgcccccaacaacacctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatacgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtacaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgataactctacaaaattgttttggtttaatccatcttcttttgaaactgagggtcagtttactctgattggcatagtactgggtctggctatttacaataacactatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagaataatacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaa

SEQ ID NO: 18 - protein, 892 aa, Artificial Sequence
  Description of Artificial Sequence Synthetic polypeptide
MYRMQLLSCIALSLALVTNSMATACKRSGEPQSDDIEASRMKRAAAKHLIERYYHQLTEGCGNEACTNEFCASCPTFLRMDNNTAAIKALELYKINAKLCDPHPSKKGASSAYLENSTGAPNNTCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNTQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSTNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDNSTKLFWFNPSSFETEGQFTLIGIVLGLAIYNNTILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQNNTGTDRAPVGGLGKLKMIIAKNGPDTERLPTSHTCFNVLLLPEYSSKEKLKERLLKAITYAKGFGML

SEQ ID NO: 19 - DNA, 2613 bp, Mus sp.
atggccacagcttgtaaaagatcaccaggagaatcccagtctgaggacattgaagctagccgaatgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgatgaagctacgaaattattttggtttaatccatcttcttttgaaactgagggtcagtttactctgattggcatagtcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagtttacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaa

SEQ ID NO: 20 - protein, 870 aa, Mus sp.
MATACKRSPGESQSEDIEASRMKRAAAKHLIERYYHQLTEGCGNEACTNEFCASCPTFLRMDNNAAAIKALELYKINAKLCDPHPSKKGASSAYLENSKGASNNSEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNVQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSRNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATKLFWFNPSSFETEGQFTLIGIVLGLAIYNNCILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 21 - DNA, 2613 bp, Mus sp.; identical to SEQ ID NO: 19

SEQ ID NO: 22 - protein, 870 aa, Mus sp.; identical to SEQ ID NO: 20

SEQ ID NO: 23 - DNA, 2550 bp, Mus sp.
atgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgatgaagctacgaaattattttggtttaatccatcttcttttgaaactgagggtcagtttactctgattggcatagtcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagtttacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaa

SEQ ID NO: 24 - protein, 849 aa, Mus sp.
MKRAAAKHLIERYYHQLTEGCGNEACTNEFCASCPTFLRMDNNAAAIKALELYKINAKLCDPHPSKKGASSAYLENSKGASNNSEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNVQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSRNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDEATKLFWFNPSSFETEGQFTLIGIVLGLAIYNNCILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 25 - DNA, 2673 bp, Artificial Sequence
  Description of Artificial Sequence Synthetic polynucleotide
atgtacaggatgcaactcctgtcttgcattgcactaagtcttgcacttgtcacaaacagtatggccacagcttgtaaaagatcaccaggagaatcccagtctgaggacattgaagctagccgaatgaagcgagcagctgcaaagcatctaatagaacgctactaccatcagttaactgagggctgtggaaatgaggcctgcacgaatgagttttgtgcttcctgtccaacttttcttcgtatggataacaatacagcagctattaaagcccttgagctttataaaattaatgcaaaactctgtgatcctcatccctccaagaaaggagcaagctcagcttaccttgagaactcaacaggtgcatctaacaacacagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatacacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagcacaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgataataatacgaaattattttggtttaatccatcttcttttgaaactgagggtcagtttactctgattggcatagtcctgggtctggctatttacaataatactatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagaatacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaa

SEQ ID NO: 26 - protein, 890 aa, Artificial Sequence
  Description of Artificial Sequence Synthetic polypeptide
MYRMQLLSCIALSLALVTNSMATACKRSPGESQSEDIEASRMKRAAAKHLIERYYHQLTEGCGNEACTNEFCASCPTFLRMDNNTAAIKALELYKINAKLCDPHPSKKGASSAYLENSTGASNNTEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNTQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSTNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDNNTKLFWFNPSSFETEGQFTLIGIVLGLAIYNNTILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQNTTGTDRAPVGGLGKLKMIIAKNGPDTERLPTSHTCFNVLLLPEYSSKEKLKERLLKAITYAKGFGML

SEQ ID NO: 27 - DNA, 2673 bp, Artificial Sequence; identical to SEQ ID NO: 25
  Description of Artificial Sequence Synthetic polynucleotide

SEQ ID NO: 28 - protein, 890 aa, Artificial Sequence; identical to SEQ ID NO: 26
  Description of Artificial Sequence Synthetic polypeptide

SEQ ID NO: 29 - DNA, 2610 bp, Artificial Sequence
  Description of Artificial Sequence Synthetic polynucleotide
atgtacaggatgcaactcctgtcttgcattgcactaagtcttgcacttgtcacaaacagtatgaagcgagcagctgcaaagcatctaatagaacgctactaccatcagttaactgagggctgtggaaatgaggcctgcacgaatgagttttgtgcttcctgtccaacttttcttcgtatggataacaatacagcagctattaaagcccttgagctttataaaattaatgcaaaactctgtgatcctcatccctccaagaaaggagcaagctcagcttaccttgagaactcaacaggtgcatctaacaacacagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatacacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagcacaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtagatgagggaggcgtttccaaagagttttttcagttggttgtggaggaaatttttaatccagatattggtatgttcacatatgataataatacgaaattattttggtttaatccatcttcttttgaaactgagggtcagtttactctgattggcatagtcctgggtctggctatttacaataatactatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagaatacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaa

SEQ ID NO: 30 - protein, 869 aa, Artificial Sequence
  Description of Artificial Sequence Synthetic polypeptide
MYRMQLLSCIALSLALVTNSMKRAAAKHLIERYYHQLTEGCGNEACTNEFCASCPTFLRMDNNTAAIKALELYKINAKLCDPHPSKKGASSAYLENSTGASNNTEIKMNKKEGKDFKDVIYLTEEKVYEIYEFCRESEDYSPLIRVIGRIFSSAEALVLSFRKVKQHTKEELKSLQEKDEDKDEDEKEKAACSAAAMEEDSEASSSRMGDSSQGDNNTQKLGPDDVTVDIDAIRRVYSSLLANEKLETAFLNALVYLSPNVECDLTYHNVYTRDPNYLNLFIIVMENSNLHSPEYLEMALPLFCKAMCKLPLEAQGKLIRLWSKYSADQIRRMMETFQQLITYKVISNEFNSTNLVNDDDAIVAASKCLKMVYYANVVGGDVDTNHNEEDDEEPIPESSELTLQELLGDERRNKKGPRVDPLETELGVKTLDCRKPLISFEEFINEPLNDVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDNNTKLFWFNPSSFETEGQFTLIGIVLGLAIYNNTILDVHFPMVVYRKLMGKKGTFRDLGDSHPVLYQSLKDLLEYEGSVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRESVVIREFWEIVHSFTDEQKRLFLQNTTGTDRAPVGGLGKLKMIIAKNGPDTERLPTSHTCFNVLLLPEYSSKEKLKERLLKAITYAKGFGML

SEQ ID NO: 31 - DNA, 2640 bp, Homo sapiens
ctggtggagaccagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaa

SEQ ID NO: 32 - protein, 870 aa, Homo sapiens
LVETRSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 33 - protein, 9 aa, Homo sapiens
NKTKQNKTK

SEQ ID NO: 34 - DNA, 523 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggtttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 35 - DNA, 6612 bp, Artificial Sequence
  Description of Artificial Sequence Synthetic polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggtttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgcccttgttaacagatcccccgggtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

(19) United States
(12) Patent Application Publication
(10) Pub. No.: US 2022/0305098 A1
(43) Pub. Date: Sep. 29, 2022

(54) UBE3A FOR THE TREATMENT OF ANGELMAN SYNDROME

(71) Applicant: The Regents of the University of California, Oakland, CA (US)

(72) Inventor: Joseph Anderson, Davis, CA (US)

(73) Assignee: The Regents of the University of California, Oakland, CA (US)

(21) Appl. No.: 17/636,839

(22) PCT Filed: Aug. 21, 2020
(86) PCT No.: PCT/US20/47505

§ 371 Date: Feb. 18, 2022

Related U.S. Application Data
(60) Provisional application No. 62/890,364, filed on Aug. 22, 2019.
(60) Provisional application No. 62/945,062, filed on Dec. 6, 2019.

(51) Int. Cl. A61K 38/53; A61P 25/28; C12N 5/0789; C12N 15/86; C12N 9/10

(52) U.S. Cl. CPC A61K 38/53; A61P 25/28; C12N 5/0647; C12N 15/86; C12N 9/104; C12Y 203/02; C12N 2740/15043

(57) ABSTRACT

Angelman syndrome is a genetic neurological disorder with characteristics including delayed development, intellectual disability, severe speech impairment, and problems with movement and balance. Provided herein are polynucleotides, vectors, polypeptides, cells, compositions, kits and methods to treat Angelman syndrome.

### CROSS-REFERENCE TO RELATED APPLICATIONS

This application claims priority under 35 U.S.C. § 119(e) of U.S. Provisional Application Nos. 62/890,364 and 62/945,062, filed Aug. 22, 2019 and Dec. 6, 2019, respectively, the contents of each of which is incorporated by reference in its entireties into the present application.

### SEQUENCE LISTING

The instant application contains a Sequence Listing which has been submitted electronically in ASCII format and is hereby incorporated by reference in its entirety. Said ASCII copy, created on Sep. 21, 2020, is named “060933_1080_SL.txt” and is 158,469 bytes in size.

### BACKGROUND

Angelman syndrome (AS) is a genetic neurological disorder with characteristics including delayed development, intellectual disability, severe speech impairment, and problems with movement and balance. Most patients have recurrent seizures and a smaller head size. Most patients display delayed development and other common symptoms appear in early childhood. Children with AS typically have a happy and excitable demeanor. Other symptoms include hyperactivity, a short attention span, and a fascination with water. As patients get older, people with Angelman syndrome become less excitable and sleeping problems improve. However, these patients continue to have intellectual disability, speech impairment, and seizures for the rest of their lives.

AS is caused by the loss of function of a gene called UBE3A. People inherit one copy of the UBE3A gene (“Ube3a”) from each parent. Both copies of Ube3a are active in many of the body's tissues. In the brain, however, only the maternal copy is active. This parent-specific activation of a gene is caused by a process called genomic imprinting. If the maternal copy of UBE3A is lost because of a deletion or mutation, a person will lack expression of Ube3a in some parts of the brain. No effective therapies are available to treat AS. This disclosure provides a gene therapy to replace or diminish the symptoms and causes of AS.

### SUMMARY OF THE DISCLOSURE

Thus, in one aspect, provided herein is a recombinant polynucleotide encoding a Ubiquitin Protein Ligase E3A (Ube3a) protein having one or more naturally occurring or non-occurring glycosylation sites, for example, for use in gene therapy and research. In one aspect, the Ube3a protein has one or more naturally occurring or non-naturally occurring glycosylation sites, or two or more naturally occurring or non-naturally occurring glycosylation sites, or three or more naturally occurring or non-naturally occurring glycosylation sites. In another aspect, the Ube3a protein has four or more naturally occurring or non-naturally occurring glycosylation sites. In another aspect, the Ube3a protein has five or more naturally occurring or non-naturally occurring glycosylation sites. In another aspect, the Ube3a protein has six or more naturally occurring or non-naturally occurring glycosylation sites. In one aspect, the Ube3a protein has seven or more, or eight or more naturally occurring or non-naturally occurring glycosylation sites. In one aspect, the Ube3a protein has naturally occurring and non-naturally occurring glycosylation sites. In one aspect, the Ube3a protein is non-naturally occurring as disclosed herein, or an equivalent or complement thereof. In one aspect, the Ube3a protein is naturally occurring but contains one or more naturally occurring or non-naturally occurring glycosylation sites. In another aspect, the protein is naturally occurring but in each aspect, the protein has one or more non-naturally occurring glycosylation sites. In one aspect, the protein is a non-naturally occurring Ube3a protein or polypeptide having one or more non-naturally occurring glycosylation sites. Additionally or alternatively, one or more of the glycosylation sites are non-naturally occurring.

Also provided are Ube3a proteins having one or more glycosylation sites, or two or more glycosylation sites, or three or more glycosylation sites. In another aspect, the Ube3a protein has four or more glycosylation sites. In another aspect, the Ube3a protein has five or more glycosylation sites. In another aspect, the Ube3a protein has six or more glycosylation sites. In one aspect, the Ube3a protein has seven or more, or eight or more glycosylation sites. In one aspect, the protein is non-naturally occurring and contains one or more glycosylation sites. In another aspect, the protein is naturally occurring but in each aspect, the protein has one or more glycosylation sites. In one aspect, the protein is a non-naturally occurring Ube3a protein or polypeptide having one or more non-naturally occurring glycosylation sites. Additionally or alternatively, one or more of the glycosylation sites are non-naturally occurring. In a further aspect, the proteins further comprise a cell penetrating domain. In yet a further aspect, the proteins comprise a secretion signal. Additionally or alternatively, the protein further comprises a detectable or purification marker.

In one aspect, the protein is encoded by a polynucleotide provided herein or its complement or equivalent thereof, for example, in the Sequence Listing section of this document as well as equivalents of each thereof. In one aspect, the polynucleotides also are non-naturally occurring and may optionally comprise a polynucleotide encoding a cell penetrating domain.

In a further aspect, the polynucleotide further comprises a promoter operatively linked to the polynucleotide. Non-limiting examples of such include pol II promoters selected from the group of an MNDU3 promoter, a CMV promoter, a PGK promoter, and an EF1alpha promoter. The promoter can be operatively linked to the coding polynucleotides to drive expression in a suitable host system. In a further aspect, the polynucleotide further comprises a polynucleotide encoding a secretion signal located 5′ to the polynucleotide encoding the modified Ube3a protein. Non-limiting examples of secretion signals include a single chain fragment variable secretion signal, a twin-arginine transport protein secretion signal, an IL-4 secretion signal, an IL-2 secretion signal and an IL-10 secretion signal. An exemplary secretion signal polynucleotide is provided below along with equivalents thereof. The polynucleotides can further comprise a polynucleotide that is, or encodes a detectable or purification marker.

Also provided are vectors comprising the recombinant polynucleotides as described herein. The vectors include vectors for expression in prokaryotic and eukaryotic host cell systems, e.g., a plasmid, or a viral vector such as, baculovirus, a retroviral vector, an adenoviral vector, an AAV vector, or a lentiviral vector. Exemplary vector maps are shown in FIGS. 3A to 3D. In some embodiments, the recombinant polynucleotide is flanked by inverted terminal repeats (ITRs) of viral vectors, e.g., adenoviral or lentiviral vectors.

Non-viral vectors may include a plasmid that comprises a heterologous polynucleotide capable of being delivered to a target cell, either in vitro, in vivo or ex-vivo. The heterologous polynucleotide can comprise the mutated Ube3a gene (such as a recombinant polynucleotide as disclosed herein) and can be operably linked to one or more regulatory elements and may control the transcription of the mutated Ube3a gene. As used herein, a vector need not be capable of replication in the ultimate target cell or subject. The term vector may include expression vector and cloning vector. In one aspect, the vector is the pCCLc plasmid vector.

The polynucleotides and vectors can be contained in a host cell system for delivery or expression of the polynucleotides. The cell can be a prokaryotic or a eukaryotic cell. In one aspect, the host cell is a mammalian cell, e.g., a canine, feline, bovine, equine, murine, rat or human. The mammalian cell can be selected from a stem cell, e.g., an induced pluripotent stem cell (iPSC), an embryonic stem cell, an adult or somatic stem cell. In one aspect, the stem cell is a mesenchymal stem cell such as for example, a hematopoietic stem cell or a neuronal stem cell. In another aspect, the stem cell is a mesenchymal stem cell optionally identified by expressing the CD34+ marker.

Also provided are populations of the cells, that can be heterologous (of different species or having different vectors and polynucleotides) or substantially homogeneous as well as clonal.

Further provided are compositions comprising one or more of the polynucleotides, protein or polypeptide, vectors, host cells or populations as described herein, and a carrier. In one aspect, the carrier is a pharmaceutically acceptable carrier.

Also provided is a viral packaging system comprising: (a) the viral vector as described herein; (b) a packaging plasmid; and (c) an envelope plasmid. In a further aspect, the system further comprises (d) a packaging cell line, such as, for example, the HEK-293 cell line. The packaging system can be used to transduce a packaging cell line under conditions suitable to package the viral vector.

Also provided is a method to express a secreted modified Ube3a protein comprising, or alternatively consisting essentially of, or yet further consisting of, growing the host cell as described herein under conditions that allow for the expression of the Ube3a protein. The method can be practiced in vitro, ex vivo or in vivo. Further provided is a method to express modified Ube3a in a subject, comprising or alternatively consisting essentially of, or yet further consisting of, administering an effective amount of the vector or host cell as described herein to the subject, thereby expressing modified Ube3a in the subject. In one aspect, the subject is a mammal, e.g., a human patient. In a further aspect, the subject is deficient or carries a defective Ube3a gene. In another aspect, the mammal is asymptomatic for Angelman syndrome. In another aspect, the subject is a fetus, an infant or a pre-pubescent subject that is or is not, symptomatic for Angelman syndrome (which is also referred to herein as AS). In an additional aspect, the subject is an adult, optionally, an adult human, further optionally, an adult human greater than 18 years of age.

Further provided is a method to treat Angelman syndrome in a subject carrying a defective Ube3a gene or allele, the method comprising, or alternatively consisting essentially of, or yet further consisting of, administering, for example an effective amount of, one or more of: the polynucleotide, the vector, the host cell, or the Ube3a protein or polypeptide as described herein to the subject, thereby treating Angelman syndrome. In a further aspect, the subject is deficient or carries a defective Ube3A gene. In one aspect, the subject is a mammal, e.g. a human patient. In one aspect, the mammal is symptomatic for Angelman syndrome. In another aspect, the mammal is asymptomatic for Angelman syndrome. In another aspect, the subject is a fetus, an infant or a pre-pubescent subject that is or is not, symptomatic for AS. In an additional aspect, the subject is an adult, optionally, an adult human, further optionally, an adult human greater than 18 years of age.

Further provided is a kit comprising one or more of: the polynucleotide, the protein or polypeptide, the vector, the host cell, or the population as described herein and optionally, instructions for use.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1F show schematic of Ube3a expressing lentiviral vectors. FIG. 1A depicts a self-inactivating lentiviral vector, CCLc-X backbone was used. It is a parental vector, without the transgene expressing Ube3a polynucleotide. An exemplified sequence of a CCLc-MNDU3-X vector is provided as SEQ ID NO: 35. FIG. 1B depicts an EGFP vector used as an empty vector control. FIG. 1C shows that the modified mouse Ube3a isoform 3 was cloned under the control of the MNDU3 promoter. EGFP was cloned downstream under the control of a PGK promoter. FIG. 1D shows that the modified human isoform 1 was cloned under the control of the MNDU3 promoter. EGFP was cloned downstream under the control of a PGK promoter. FIG. 1E shows that the modified human isoform 1 was cloned under the control of the MNDU3 promoter without the EGFP reporter gene. FIG. 1F shows the exemplary vector of FIG. 1E and notes the secretion signal (ss) therein.

FIG. 2 shows CFU assay of Ube3a vector transduced human CD34+ HPC. Human CD34+ HPC were left nontransduced (NT, left column of each bar group) or transduced with either the EGFP-alone control vector (EGFP, middle column of each bar group) or the Ube3a vector (Ube3a, right column of each bar group). Cells were then cultured in methylcellulose media for 12 days when specific colonies (BFU-E, GM, and GEMM) were counted.

FIGS. 3A-3B provide overexpression and ubiquitination activity of the Ube3a lentivector. Human CD34+ HPC were transduced with the Ube3a lentivector (Ube3a) and derived into mature macrophages. Cell extracts were then generated and evaluated for (FIG. 3A) overexpression of Ube3a by Western blot and (FIG. 3B) ubiquitination activity of the Ube3a S5a target protein. Control nontransduced (NT) and EGFP vector alone (EGFP) transduced cells were used as controls. FIG. 3B further shows that different mutated forms of Ube3a ubiquitinate their S5A target. “AS Native” denotes using the nonsecreted wild type form of Ube3a which is naturally found in cells as the testing sample. “AS WT” denotes using the wild type form of Ube3a with the addition of the IL-2 secretion signal as the testing sample. “AS4” denotes using a mutated form of Ube3a with 4× mutated sites to create 4× potential glycosylation sites in addition to the IL-2 secretion signal as the testing sample. “AS8” denotes using a mutated form of Ube3a with 8× mutated sites to create 8× potential glycosylation sites in addition to the IL-2 secretion signal as a testing sample. “Ubiquitinated S5A” denotes bands of the ubiquitinated form of S5A which increases in molecular weight with increased ubiquitination.

FIG. 4 illustrates an exemplary study design for correction of phenotypes in newborn mice.

FIGS. 5A-5H show that locomotor ability, balance, motor coordination and gait are measured by a tailored motor behavioral battery. Rigorous assessment of motor translational phenotypes using four standard motor behavioral tests in treated and untreated Ube3a mice that were irradiated and transplanted as pups with either non-transduced (NT Het, open dots in FIG. 5B and dots having solid left halves in other panels) or the Ube3a lentivector transduced (Ube3a Het, dashed line in FIG. 5B or open dots in other panels) human CD34+ HSC. Wild type mice (WT, solid black dots) were used as a control. Eight weeks post-transplant, mice were subjected to open field locomotion (FIG. 5A, horizontal; FIG. 5B, vertical, and FIG. 5C, total activity), balance beam (FIG. 5D and FIG. 5E), rotarod (FIG. 5F), and treadmill walking and DigiGait analysis (FIG. 5G and FIG. 511). In all tests, Ube3a-deficient mice transplanted with the Ube3a vector transduced human CD34+ HSC (Ube3a Het) exhibited wildtype values of performance. Open field activity was increased by both the total distance and horizontal activity metrics. Beams decrease in width and are more difficult to cross going from Rod #1 to Rod #2 to Rod #3. Latency to fall from the rotarod was significantly improved in the Ube3a-deficient mice transplanted with Ube3a vector transduced human CD34+ HSC (Ube3a Het) compared to the nontransplanted (NT Het) cells controls. In AS patients, standard AS mice and the novel Ube3a mice also exhibit unusually wide stances. DigiGait analysis showed a narrowing of these wide stances in the treated group. *=p<0.05.

FIGS. 6A-6B provide results obtained from the novel object recognition assay. Eleven weeks post-transplant, mice were assessed for learning and memory abilities using the novel object recognition test. FIG. 6A provides evaluation of time sniffing the novel object and the familiar object. FIG. 6B provides results of familiarization test with two identical (familiar) objects. Newborn BGU mice were transplanted with either non-transduced (NT-HET) or Ube3a vector transduced (Ube3a-HET) human CD34+ HSC. Wild type mice (WT) and nontransplanted BGU mice (HET) were used as controls. Ube3a-HET exhibited intact object recognition similar to WT while, NT-HETS did not spend more time with the novel object, exhibiting a lack of recognition memory. * p<0.05, novel versus familiar.

FIGS. 7A-7B show rescue of elevated delta power detected in Ube3a-HET mice (FIG. 7A) and in comparison to an EEG of human (FIG. 7B, reproduced from Anderson, B C M, 2017). Surface EEG was collected in mice via a wireless telemetric device and analyzed for spectral power differences. Delta power peaks are displayed in HET and NT-HET animals that are not present in WT or Ube3a-HET treated animals. The elevated delta phenotype observed in HET animals recapitulates elevated delta seen clinically and re-expression of UBE3A resulted in rescue to WT levels. *p<0.0001.

FIG. 8 shows expression of Ube3a in the brains of Ube3a vector transduced cell transplanted mice. WT, Ube3a−/+ BGU (HET), and Ube3a vector transduced cell transplanted Ube3a−/+(Ube3a-HET) mice were euthanized and analyzed for Ube3a expression using a DAB peroxidase substrate and an anti-Ube3a antibody. Bright field immunohistochemistry stained slides were scanned and relative Ube3a expression was measured. *p=0.0126.

FIGS. 9A-9C show engraftment and development of human T cells in NRG mice. Human CD34+ HSC were left nontransduced or transduced with either the EGFP control (EGFP) or Ube3a expressing (hAS8-EGFP) lentiviral vector. Cells were transplanted into 2-5-day-old NRG mice. At 16 weeks post-transplant, mice were euthanized and human T cells were analyzed for CD3, CD4 and CD8 expression in the (FIG. 9A) peripheral blood, (FIG. 9B) spleen, and (FIG. 9C) thymus.

FIGS. 10A-10B show engraftment and development of human B cells in NRG mice: Human CD34+ HSC were left nontransduced or transduced with either the EGFP control (EGFP) or Ube3a expressing (hAS8-EGFP) lentiviral vector. Cells were transplanted into 2-5-day-old NRG mice. At 16 weeks post-transplant, mice were euthanized and human B cells were analyzed for CD45 and CD19 in the (FIG. 10A) spleen and (FIG. 10B) bone marrow.

FIG. 11 shows engraftment and development of human macrophages and CD34+ cells in the bone marrow of NRG mice. Human CD34+ HSC were left nontransduced or transduced with either the EGFP control (EGFP) or Ube3a expressing (hAS8-EGFP) lentiviral vector. Cells were transplanted into 2-5-day-old NRG mice. At 16 weeks post-transplant, mice were euthanized and human macrophages and CD34+ cells were analyzed for engraftment in the bone marrow.

FIG. 12 illustrates an exemplary study design for correction of phenotypes in adult mice.

FIGS. 13A-13G show that locomotor ability, balance, motor coordination, and gait are measured by a tailored motor behavioral battery with adult BGU mice transplanted with Ube3a lentiviral vector transduced human CD34+ HSC. Rigorous assessment of motor translational phenotypes using four standard motor behavioral tests in treated and untreated Ube3a mice that were irradiated and transplanted as adults with either nontransduced (NT-HET) or the Ube3a lentivector (Ube3a-HET) transduced human CD34+ HSC. Four to six week old mice were conditioned with busulfan and transplanted intravenously. Six weeks later mice were subjected to open field locomotion (FIG. 13A, horizontal; FIG. 13B, vertical, and FIG. 13C, total activity), balance beam (FIG. 13D and FIG. 13E), rotarod (FIG. 13F) and treadmill walking (FIG. 13G). In all tests, Ube3a-deficient mice transplanted with the Ube3a vector transduced human CD34+ HSC (Ube3a-HET) exhibited wildtype values of performance. Open field activity was increased by both the total distance and horizontal activity metrics. Beams decrease in width and are more difficult to cross going from Rod #1 to Rod #2 to Rod #3. Wild type mice (WT) were used as a control. Latency to fall from the rotarod was significantly improved in the Ube3a-deficient mice transplanted with Ube3a vector transduced human CD34+ HSC (Ube3a-HET) compared to the non-transduced (NT-HET) cells and non-transplanted (HET) controls. In AS patients, standard AS mice and the novel Ube3a mice also exhibit unusually wide stances. DigiGait analysis showed a narrowing of these wide stances in the treated group. *=p<0.05.

FIGS. 14A-14B show novel object recognition assay with BGU mice transplanted with Ube3a lentiviral vector transduced human CD34+ HSC. Six weeks post-transplant in adult mice, subjects were assessed for learning and memory abilities using the novel object recognition test. FIG. 14A shows evaluation of time sniffing the novel object and the familiar object. FIG. 14B provides results of familiarization test with two identical (familiar) objects. Ube3a HET mice were transplanted with either nontransduced (NT Het) or Ube3a vector transduced (Ube3a Het) human CD34+ HSC or left nontransplanted (HET). Wild type mice (WT) were used as a control. Transplanted HETs (Ube3a-HET) exhibited intact object recognition similar to WT while, NT-HETs and HETs did not spend more time with the novel object, exhibiting a lack of recognition memory. * p<0.05, novel versus familiar.

FIG. 15 shows expression of Ube3a in the brains of Ube3a−/+ adult mice. WT, Ube3a−/+ BGU (HET), and Ube3a−/+ mice transplanted with nontransduced (NT-HET) or Ube3a vector transduced (Ube3a-HET) cells were euthanized and analyzed for Ube3a expression using a DAB peroxidase substrate and an anti-Ube3a antibody. Bright field immunohistochemistry stained slides were scanned and Ube3a positive cells were counted. *p=0.0058.

FIGS. 16A-16B provide examples of several glycosylation sites based on a nucleotide fragment (as reproduced in SEQ ID NO: 31) of Homo sapiens ubiquitin protein ligase E3A (UBE3A), transcript variant 5, having an NCBI Reference No. of NM_001354506, and its translated amino acid sequences (SEQ ID NOs: 32 and 33). The amino acid sequence numbering is based on SEQ ID NO: 8, which is a fragment of SEQ ID NO: 32. Non-capitalized amino acid residues provide N-glycosylation sites predicated by an online tool named NetNGlyC. Bold, italic and shaded amino acid residues provide examples of sites containing an S or a T where mutation of the second amino acid on the N-terminal end to an N would result in a potential glycosylation site. Underlined amino acid residues provide examples of sites which contain an N where mutation of the second amino acid on the C terminal end to an S/T would result in a potential glycosylation site. Boxed amino acid residues indicate some exemplified glycosylation sites that could be created by mutation as identified herein. The coding sequence (CDS) of the nucleotide fragment starts with a start codon (marked as shaded ATG) and ends with a stop codon (marked as shaded TAA). The rest shaded nucleotide residues provide potential mutation site to create a glycosylation site. FIG. 16B further illustrates how the mutation can be performed to form a glycosylation site in two fragments of the amino acid sequence shown in FIG. 16A. The fragment on the top is amino acid (aa) 201 to aa 220 of SEQ ID NO: 32, i.e., aa 183 to aa 202 of SEQ ID NO: 8, while the fragment on the bottom is aa 341 to aa 360 of SEQ ID NO: 32, i.e., aa 323 to aa342 of SEQ ID NO: 8.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST. In some embodiments, the polynucleotide as disclosed herein is a RNA. In some embodiments, the polynucleotide as disclosed herein is a DNA. In some embodiments, the polynucleotide as disclosed herein is a hybrid of DNA and RNA.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, and/or a reverse-complement reference, optionally under conditions of high stringency.

Additionally or alternatively, an equivalent nucleic acid, polynucleotide or oligonucleotide is one having at least 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement. In one aspect, the equivalent must encode functional protein that optionally can be identified through one or more assays described herein. In another aspect, an equivalent has at least the 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement, with the proviso that one or more mutated polynucleotides identified herein having one or more non-naturally occurring glycosylation sites are not mutated from the corresponding mutated polynucleotides in the disclosed sequences. For example, an equivalent polynucleotide of modified human isoform #1 would have modifications of the nucleotides positions with the exception of one or more nucleotides at positions selected from 190, 293, 310, 661, 662, 1066, 1067, 1771, 1773, 1870, 1871, 2413, 2414, 2417, 2418, as shown in the below sequence listing. In addition or alternatively, the equivalent of a polynucleotide would encode a protein or polypeptide of the same or similar function as the reference or parent polynucleotide.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term equivalent and biological equivalent are used interchangeably, for example when referring to a protein or polypeptide as a reference. In some embodiments, an equivalent protein or polypeptide is one having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to a polypeptide or protein as disclosed herein. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to polypeptide or protein encoded by an equivalent polynucleotide as noted herein. In addition or alternatively, the equivalent of a polynucleotide would encode a protein or polypeptide of the same or similar function as the reference or parent polynucleotide.

In some embodiments, the equivalent is a functional protein that optionally can be identified through one or more assays described herein. In another aspect, an equivalent has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. Some embodiments are with the proviso that one or more amino acids identified herein as mutated to a possible glycosylation site are mutated from a polypeptide or protein in the disclosed sequences. Some embodiments are with the proviso that one or more amino acids identified herein as mutated to a possible glycosylation site are not mutated from a polypeptide or protein in the disclosed sequences. For example, an equivalent polypeptide of modified human isoform #1 would have modifications of the amino acids positions with the exception of one or more of the amino acids at positions selected from 64, 98, 104, 221, 356, 591, 624, 805 and 806, as shown sequences as disclosed herein.

In some embodiments, the equivalent protein or polypeptide performs functions similar to a wildtype and/or at a similar level compared to a wildtype. For example, a biological equivalent of Ube3a protein or polypeptide may have similar functions compared to a wild type Ube3a protein and/or any one or more of the functions of the biological equivalent of Ube3a protein or polypeptide at a similar level (such as having similar activity) compared to a wildtype Ube3a protein or polypeptide. In further embodiments, for example, the equivalent's function is at a level of at least about 50%, at least about 60%, at least about 70%, at least about 80%, at least about 90%, at least about 95%, at least about 100%, at least about 1.5 folds, at least about 2 folds, at least about 3 folds, at least about 5 folds, at least about 10 folds of those wild type ones. A non-limiting example of such functions include a ubiquitination activity, such as ubiquitinating a Ube3a target protein S5a. In some embodiments, the equivalent has a ubiquitination activity that is at least about 50%, at least about 60%, at least about 70%, at least about 80%, at least about 90%, at least about 95%, at least about 100%, at least about 1.5 folds, at least about 2 folds, at least about 3 folds, at least about 5 folds, at least about 10 folds of the wild type one. Exemplified methods of evaluating such activity are known in the art and several are exemplified in the Experimental Methods.

In some embodiments, a wildtype polynucleotide, polypeptide or protein (which is also referred to herein as a wildtype) means a naturally occurring polynucleotide, polypeptide or protein. In further embodiments, a wildtype Ube3a protein or polypeptide comprises, or alternatively consists essentially of, or yet further consists of a sequence selected from any one or more of SEQ ID NOs: 8, 10, 12, 20, 22 and/or 24, or a natural variant thereof. Variable such natural variants are listed on www.uniprot.org/uniprot/Q05086 and www.uniprotorg/uniprot/008759, each of which is incorporated herein by its entirety. In some embodiments, a wildtype Ube3a protein or polypeptide is an isoform comprising, or alternatively consisting essentially of, or yet further consisting of a sequence selected from any one or more of SEQ ID NOs: 8, 10, 12, 20, 22 and/or 24. In further embodiments, a wildtype Ube3a polynucleotide comprises, or alternatively consists essentially of, or yet further consists of a sequence selected from any one or more of SEQ ID NOs: 7, 9, 11, 19, 21 and/or 23, or a natural variant thereof. Variable such natural variants are listed on www.genecards.org/cgi-bin/carddisp.pl?gene=UBE3A as transcripts or variants, which is incorporated herein by its entirety. In some embodiments, a wildtype Ube3a polynucleotide is an isoform comprising, or alternatively consisting essentially of, or yet further consisting of a sequence selected from any one or more of SEQ ID NOs: 7, 9, 11, 19, 21 and/or 23.

As used herein, a natural variant refers to a mutant that is naturally generated (for example, incidentally generated) instead of being generated by artificial means.

In some embodiments, a natural variant is functional, for example, performing functions similar to a wildtype and/or at a similar level compared to a wildtype. For example, a natural variant of Ube3a protein or polypeptide may have similar functions compared to a wild type Ube3a protein and/or any one or more of the functions of the natural variant of Ube3a protein or polypeptide at a similar level (such as having similar activity) compared to a wildtype Ube3a protein or polypeptide. In further embodiments, for example, the natural variant's function is at a level of at least about 50%, at least about 60%, at least about 70%, at least about 80%, at least about 90%, at least about 95%, at least about 100%, at least about 1.5 folds, at least about 2 folds, at least about 3 folds, at least about 5 folds, at least about 10 folds of those wild type ones. A non-limiting example of such functions include a ubiquitination activity, such as ubiquitinating a Ube3a target protein S5a. In some embodiments, the natural variant has a ubiquitination activity that is at least about 50%, at least about 60%, at least about 70%, at least about 80%, at least about 90%, at least about 95%, at least about 100%, at least about 1.5 folds, at least about 2 folds, at least about 3 folds, at least about 5 folds, at least about 10 folds of the wildtype one. Exemplified methods of evaluating such activity can be found in the Experimental Methods.

In some embodiments, a natural variant is not functional, therefore referred to herein as a defective variant, gene or allele. For example, such defective gene or allele encodes a defective protein variant not performing certain functions of a wildtype and/or at a substantially reduced level compared to a wildtype. In some embodiment, the defective protein variant's function is at a level of less than about 50%, less than about 25%, less than about 20%, less than about 10%, less than about 5%, less than about 2%, or less than about 1% of those wild type ones. A non-limiting example of such functions include a ubiquitination activity, such as ubiquitinating a Ube3a target protein S5a. In some embodiments, the defective variant has a ubiquitination activity that is less than about 50%, less than about 25%, less than about 20%, less than about 10%, less than about 5%, less than about 2%, or less than about 1% of those wild type ones. Exemplified methods of evaluating such activity can be found in the Experimental Methods.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “probe” when used in the context of polynucleotide manipulation refers to an oligonucleotide that is provided as a reagent to detect a target potentially present in a sample of interest by hybridizing with the target. Usually, a probe will comprise a detectable label or marker or a means by which a label or marker can be attached, either before or subsequent to the hybridization reaction. Alternatively, a “probe” can be a biological compound such as a polypeptide, antibody, or fragments thereof that is capable of binding to the target potentially present in a sample of interest.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

Unmodified cells are sometimes referred to as “source cells” or “source stem cells”. The cells may be prokaryotic or eukaryotic, and include but are not limited to bacterial cells, yeast cells, plant cells, insect cells, animal cells, and mammalian cells, e.g., felines, canines, equines, murines, rats, simians, bovines, porcines and humans.

In one embodiment, an “immature cell” refers to a cell which does not possess the desired (adult) phenotype or genotype. For example, in one embodiment, a mature cell is a cell that is being replaced. The immature cell can be subjected to techniques including physical, biological, or chemical processes which changes, initiates a change, or alters the phenotype or genotype of the cell into a “mature cell.” A “mature cell” refers to a cell which possess the desired phenotype or genotype.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

As used herein, an “antibody” includes whole antibodies and any antigen-binding fragment or a single chain thereof. Thus, the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present disclosure. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and CH, domains; a F(ab′)2 fragment, a bivalent fragment comprising two Fab fragments linked by a disulfide bridge at the hinge region; a Fd fragment consisting of the V_{H} and CH, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “antibody variant” intends to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this disclosure. Derivatives include, but are not limited to, for example, bispecific, multi specific, heterospecific, tri specific, tetraspecific, multi specific antibodies, diabodies, chimeric, recombinant and humanized.

As used herein, “Ube3a” refers to gene encoding a protein called ubiquitin protein ligase E3A. In some embodiments, the abbreviation of Ube3a refers to a protein or a polypeptide. In some embodiments, the abbreviation of Ube3a refers to a polynucleotide. Ubiquitin protein ligases are enzymes that target other proteins to be broken down (degraded) within cells. These enzymes attach a small molecule called ubiquitin to proteins that should be degraded. Cellular structures called proteasomes recognize and digest these ubiquitin-tagged proteins. Protein degradation is a normal process that removes damaged or unnecessary proteins and helps maintain the normal functions of cells. See ghr.nlm.nih.gov/gene/UBE3A, last accessed on May 23, 2019.

Studies suggest that ubiquitin protein ligase E3A plays a critical role in the normal development and function of the nervous system. Studies suggest that it helps control (regulate) the balance of protein synthesis and degradation (proteostasis) at the junctions between nerve cells (synapses) where cell-to-cell communication takes place. Regulation of proteostasis is important for the synapses to change and adapt over time in response to experience, a characteristic called synaptic plasticity. Synaptic plasticity is critical for learning and memory. There are 3 isoforms of the gene that vary at their 5′ ends (see NCBI NM_0013545606; NM_000462.5; and NM001354505). Yamamoto et al. (1997) Genomics April 15; 41(2):263-266 describes the genomic structure of the coding region of E6-AP and an analysis of a set of five E6-AP mRNAs with the potential to encode three protein isoforms of the E6-AP protein (isoforms I, II, and III) that differ at their extreme amino-termini.

As used herein, an N-linked glycosylation refers to attachment of an oligosaccharide or a glycan, which is a carbohydrate consisting of several sugar molecules, to a nitrogen atom, such as the amide nitrogen of an asparagine (Asn, N) residue of a protein or a polypeptide.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters. The sequence of the MNDU3 promoter and the sequence of an exemplary CMV promoter are provided below.

A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors. Other pol II promoter may be selected, such as from those cell specific promoters (including but not limited to a CD14 promoter, a CD3 promoter, a CD19 promoter), any blood cell lineage promoters (including but not limited to a promoter of any one of CD2, CD11b, CD11c, CD16, CD24, CD56, CD66b and CD235), and/or any other promoters that can direct protein expression in a human cell.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

A signal peptide, as used herein, refers to (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined toward the secretory pathway. In one embodiment, the signal peptide is a secretary signal.

A secretary signal intends a secretory signal peptide that allows the export of a protein from the cytosol into the secretory pathway. Proteins can exhibit differential levels of successful secretion and often certain signal peptides can cause lower or higher levels when partnered with specific proteins. In eukaryotes, the signal peptide is a hydrophobic string of amino acids that is recognized by the signal recognition particle (SRP) in the cytosol of eukaryotic cells. After the signal peptide is produced from an mRNA-ribosome complex, the SRP binds the peptide and stops protein translation. The SRP then shuttles the mRNA/ribosome complex to the rough endoplasmic reticulum where the protein is translated into the lumen of the endoplasmic reticulum. The signal peptide is then cleaved off the protein to produce either a soluble, or membrane tagged (if a transmembrane region is also present), protein in the endoplasmic reticulum. These are known in the art, and commercially available from vendors, e.g., Oxford Genetics.

Cell penetrating peptides or cell penetrating domains (CPPs) or cell penetrating domains, as used herein, refer to short peptides that facilitate cellular uptake of various molecular cargos (from small chemical molecules to nanosize particles and large fragments of DNA). A “cargo”, such as a modified protein as disclosed herein, is associated with the peptides either through chemical linkage via covalent bonds or through non-covalent interactions. The function of the CPPs are to deliver the cargo into target cells, a process that commonly occurs through endocytosis with the cargo delivered to the endosomes of living mammalian cells. In some embodiments, the target cell is a neuron. CPPs typically have an amino acid composition containing either a high relative abundance of positively charged amino acids such as lysine or arginine, or have sequences that contain an alternating pattern of polar/charged amino acids and non-polar, hydrophobic amino acids. It was previously reported that the human immunodeficiency virus transactivator of transcription (HIV-TAT) protein can be delivered to cells using a CPP.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the disclosure, is a chemically modified CPP with 2 to 35 amino acids, preferably 5 to 25 amino acids, more preferably 10 to 25 amino acids, or even more preferably 15 to 25 amino acids.

CPPs can be linked to a protein recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al. (2006) Bioconjugate Chem. 17:597-602).

CPPs also include cationic conjugates which also may be used to facilitate delivery of the proteins into the cells or tissue of interest. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids; more preferably 5 to 10 cationic amino acids or other cationic subunits.

Recombinant proteins anchoring CPP to the proteins can be generated to be used for delivery to cells or tissue.

As used herein, a cleavable peptide, which is also referred to as a cleavable linker, means a peptide that can be cleaved, for example, by an enzyme. One translated polypeptide comprising such cleavable paptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

The term “stem cell” refers to a cell that is in an undifferentiated or partially differentiated state and has the capacity for self-renewal and/or to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural or neuronal stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Embryoid bodies or EBs” are three-dimensional (3D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, the term “pluripotent gene or marker” intends an expressed gene or protein that has been correlated with an immature or undifferentiated phenotype, e.g., Oct 3/4, Sox2, Nanog, c-Myc and LIN-28. Methods to identify such are known in the art and systems to identify such are commercially available from, for example, EMD Millipore (MILLIPLEX® Map Kit).

The term “phenotype” refers to a description of an individual's trait or characteristic that is measurable and that is expressed only in a subset of individuals within a population. In one aspect of the disclosure, an individual's phenotype includes the phenotype of a single cell, a substantially homogeneous population of cells, a population of differentiated cells, or a tissue comprised of a population of cells.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A population of cells intends a collection of more than one cell that is identical (clonal) or non-identical in phenotype and/or genotype. The population can be purified, highly purified, substantially homogenous or heterogeneous as described herein.

The terms effective period (or time) and effective conditions refer to a period of time or other controllable conditions (e.g., temperature, humidity for in vitro methods), necessary or preferred for an agent or composition to achieve its intended result, e.g., the differentiation or dedifferentiation of cells to a pre-determined cell type.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., Angelman syndrome. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis or prevention.

In one embodiment, the term “disease” or “disorder” as used herein refers to a disease associated with a defective Ube3a variant or gene, such as Angelman syndrome and/or Prader-Willi syndrome, a status of being diagnosed with such disease, a status of being suspect of having such disease, or a status of at high risk of having such disease.

“Administration” or “delivery” of a cell or vector or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

An “enriched population” of cells intends a substantially homogenous population of cells having certain defined characteristics. The cells are greater than 70%, or alternatively greater than 75%, or alternatively greater than 80%, or alternatively greater than 85%, or alternatively greater than 90%, or alternatively greater than 95%, or alternatively greater than 98% identical in the defined characteristics.

The term “suffering” as it related to the term “treatment” refers to a patient or individual who has been diagnosed with or is predisposed to Angelman syndrome. A patient may also be referred to being “at risk of suffering” from a disease because they carry one or more genetic mutations. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

### Descriptive Embodiments

### Polynucleotides and Polypeptides

This disclosure provides a polynucleotide encoding a Ubiquitin Protein Ligase E3A (Ube3a) protein, polypeptide, or a biological equivalent thereof. In some embodiments, the polynucleotide is recombinant and/or isolated. In some embodiments, the Ube3a protein, polypeptide, or a biological equivalent thereof comprises one or more glycosylation sites.

Also provided is the Ube3a protein, polypeptide, or a biological equivalent thereof comprising one or more glycosylation sites. In some embodiments, the Ube3a protein, polypeptide, or a biological equivalent thereof is isolated, engineered and/or recombinant.

In some embodiments, the Ube3a protein, polypeptide, or a biological equivalent thereof is not a wildtype Ube3a protein, such as those comprising a sequence selected from SEQ ID NOs: 8, 10, 12, 20, 22 or 24, or any natural variant thereof. This Ube3a protein, polypeptide, or a biological equivalent thereof is also referred to herein as a modified Ube3a protein.

In some embodiments, the Ube3a protein, polypeptide, or a biological equivalent thereof comprises two or more glycosylation sites, or three or more glycosylation sites. In some embodiments, the Ube3a protein, polypeptide, or a biological equivalent thereof comprises four or more glycosylation sites, or five or more glycosylation sites. In some embodiments, the Ube3a protein, polypeptide, or a biological equivalent thereof at least 4 or at least 5 glycosylation sites. In some embodiments, the Ube3a protein, polypeptide, or a biological equivalent thereof comprises 6 or more, or 7 or more glycosylation sites. In some embodiments, the Ube3a protein, polypeptide, or a biological equivalent thereof comprises 8 or more glycosylation sites.

In some embodiments, any one or more of the glycosylation sites may be naturally occurring. In some embodiments, any one or more of the glycosylation sites may be non-naturally occurring. In a further embodiment, at least one of the glycosylation sites is non-naturally occurring. Additionally or alternatively, any one or any two or all three amino acid residue(s) in at least one of the glycosylation sites is or are mutated compared to a wildtype Ube3a polypeptide or protein, thereby constituting the glycosylation site.

The recombinant and/or isolated polynucleotides or Ube3a proteins, polypeptides, or biological equivalents thereof can be naturally occurring or be created, such as by mutation of an open reading frame of a wild-type polynucleotide, to include one or multiple glycosylation site(s) and/or modification(s) of a naturally occurring glycosylation site to a non-naturally occurring sequence. In some embodiments, the Ube3a protein, polypeptide, or a biological equivalent thereof comprises one or more non-naturally occurring glycosylation sites. In some embodiments, a non-naturally occurring Ube3a protein, polypeptide, or a biological equivalent thereof is also referred to herein as a modified Ube3a protein or a modified protein.

In some embodiments of any disclosure herein, the Ube3a proteins, polypeptides, or biological equivalents thereof comprises at least one non-naturally occurring glycosylation site. Such non-naturally occurring glycosylation site does not render the Ube3a proteins, polypeptides, or biological equivalents thereof unfunctional. For example, as shown in Experiment No. 1, they are still capable of ubiquitinating S5a. Further, as shown in the Experimental Methods, they are capable of effectively treating AS.

Examples of such Ube3a proteins, polypeptides, or biological equivalents thereof are provided below where the motif “NXT/S” identifies the glycosylation site where X is any amino acid residue. In some embodiments, a glycosylation site comprises, or alternatively consists essentially of, or yet further consists of a consensus sequence of NXaaT/S (i.e., NXaaT and/or NXaaS) where Xaa is any amino acid residue. In some embodiments, a glycosylation site comprises, or alternatively consists essentially of, or yet further consists of a consensus sequence of NXaaT/S (i.e., NXaaT and/or NXaaS) where Xaa is any amino acid residue except proline (P). In some embodiments, the glycosylation is N-linked.

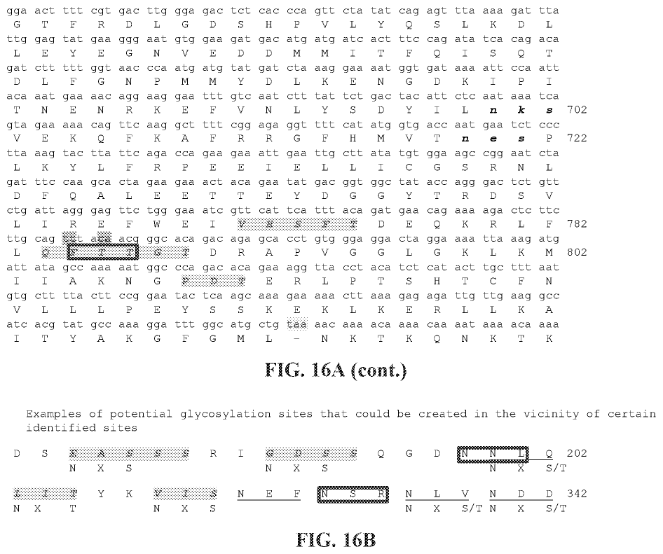

In some embodiments, a starting or reference Ube3a protein or polypeptide, such as a wild type as disclosed herein, an isoform thereof, a natural variant thereof, or a non-natural variant thereof, may be mutated to have at least one non-naturally occurring glycosylation site and one or more optional additional mutated residues that do not constitute a glycosylation site, thus resulting in an engineered and/or recombinant Ube3a protein, polypeptide or a biological equivalent thereof. In further embodiments, the starting or reference Ube3a protein or polypeptide may have an amino acid residue at any position mutated optionally to N with the proviso that the second amino acid residue on its C terminus side is a T or an S. Additionally or alternatively, the starting or reference Ube3a protein or polypeptide may have an amino acid residue at any position mutated optionally to S to T with the proviso that the second amino acid residue on its N terminus side is an N. In some embodiments, any part of the starting or reference Ube3a protein or polypeptide having a sequence of Xaa1Xaa2Xaa3 may be engineered to be NXaaT/S (i.e., NXaaT and/or NXaaS), thus resulting in a recombinant Ube3a protein, polypeptide or a biological equivalent thereof as disclosed herein, wherein Xaa1, Xaa2, Xaa3 or Xaa can be any amino acid residue. In a further embodiment, either or both of Xaa2 and Xaa is or are not P. In one embodiments, the Xaa3 is S or T and optionally the Xaa1 is not N. In another embodiment, Xaa1 is N and optionally the Xaa3 is neither S nor T. In some embodiments, Xaa1 is not N and Xaa3 is neither S nor T. Exemplified glycosylation sites and/or their positions can be found in FIG. 16A, FIG. 16B, and SEQ ID NO: 32.

In some embodiments, a recombinant Ube3a protein, polypeptide or a biological equivalent thereof can be engineered and/or produced by mutating one or more of nucleotide residues of the coding sequence of a starting or reference Ube3a protein or polypeptide. In further embodiments, such mutated nucleotide residues encode a glycosylation site. Some exemplified nucleotide mutations can be found in FIG. 16A, FIG. 16B, and SEQ ID NOs: 13, 15, 17, 25, 27, 29 and 31.

In some embodiments, the glycosylation sites are at amino acid (aa) positions of the polypeptide, protein or the equivalent thereof corresponding to one or more of those selected from the following:

aa62 to aa64 of SEQ ID NO: 14, aa96 to aa98 of SEQ ID NO: 14, aa102 to aa104 of SEQ ID NO: 14, aa219 to aa221 of SEQ ID NO: 14, aa354 to aa356 of SEQ ID NO: 14, aa591 to aa 593 of SEQ ID NO: 14, aa622 to aa624 of SEQ ID NO: 14, aa 805 to aa 807 of SEQ ID NO: 14, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa85 to aa87 of SEQ ID NO: 16, aa119 to aa121 of SEQ ID NO: 16, aa125 to aa127 of SEQ ID NO: 16, aa242 to aa244 of SEQ ID NO: 16, aa377 to aa379 of SEQ ID NO: 16, aa614 to aa616 of SEQ ID NO: 16, aa645 to aa647 of SEQ ID NO: 16, aa828 to aa830 of SEQ ID NO: 16, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa82 to aa84 of SEQ ID NO: 18, aa116 to aa118 of SEQ ID NO: 18, aa 122 to aa124 of SEQ ID NO: 18, aa239 to aa241 of SEQ ID NO: 18, aa374 to aa376 of SEQ ID NO: 18, aa611 to aa613 of SEQ ID NO: 18, aa642 to aa644 of SEQ ID NO: 18, aa825 to aa827 of SEQ ID NO: 18, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa83 to aa85 of SEQ ID NO: 26 or 28, aa117 to aa119 of SEQ ID NO: 26 or 28, aa123 to aa125 of SEQ ID NO: 26 or 28, aa 237 to aa239 of SEQ ID NO: 26 or 28, aa372 to aa374 of SEQ ID NO: 26 or 28, aa609 to aa 611 of SEQ ID NO: 26 or 28, aa640 to aa642 of SEQ ID NO: 26 or 28, aa823 to aa825 of SEQ ID NO: 26 or 28, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa62 to aa64 of SEQ ID NO: 30, aa96 to aa98 of SEQ ID NO: 30, aa102 to aa104 of SEQ ID NO: 30, aa216 to aa218 of SEQ ID NO: 30, aa351 to aa353 of SEQ ID NO: 30, aa588 to aa590 of SEQ ID NO: 30, aa619 to aa621 of SEQ ID NO: 30, or aa802 to aa804 of SEQ ID NO: 30, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids.

In some embodiments of any disclosure herein, the shifted position is also referred to herein as a position in the vicinity of the reference position. In further embodiments, the reference position is any one of those identified herein. In some embodiments, the shifted position still consists of 3 or 2 or 1 amino acid residue(s). For example, a position shifting the reference position of aa62 to aa64 of SEQ ID NO: 14 to the C terminus by 1 amino acid, would be a position corresponding to aa63 to aa65 of SEQ ID NO: 14. In some embodiments, the Ube3a polypeptide, protein or biological equivalent thereof comprising one or more of glycosylation sites (for example, the modified Ube3a protein as used herein) comprises, or alternatively consists essentially of, or yet further consists of a sequence of any one or more of SEQ ID NOs: 14, 16, 18, 26, 28, 30 or a fragment thereof. In some embodiments, the Ube3a polypeptide, protein or biological equivalent thereof comprises the one or more of glycosylation sites as identified, but is a variant of a sequence of any one or more of SEQ ID NOs: 14, 16, 18, 26, 28, 30 or a fragment thereof. In some embodiments, the Ube3a polypeptide, protein or biological equivalent thereof further comprises one or more different amino acid residue(s) at a position corresponding to any one or more of SEQ ID NOs: 14, 16, 18, 26, 28, 30 where the position is not in a glycosylation site as disclosed herein. One example is, the Ube3a polypeptide, protein or biological equivalent thereof may be created based on a Ube3a natural variant by mutating the variant's one or more amino acid residue(s) to form a glycosylation site as disclosed herein.

In some embodiments, the Ube3a polypeptide, protein or the biological equivalent thereof comprises eight glycosylation sites at an amino acid (aa) positions corresponding to the eight aa positions as identified in any one of the following (a) to (e):

(a) aa62 to aa64 of SEQ ID NO: 14, aa96 to aa98 of SEQ ID NO: 14, aa102 to aa104 of SEQ ID NO: 14, aa219 to aa221 of SEQ ID NO: 14, aa354 to aa356 of SEQ ID NO: 14, aa591 to aa 593 of SEQ ID NO: 14, aa622 to aa624 of SEQ ID NO: 14, aa 805 to aa 807 of SEQ ID NO: 14, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(b) aa85 to aa87 of SEQ ID NO: 16, aa119 to aa121 of SEQ ID NO: 16, aa125 to aa127 of SEQ ID NO: 16, aa242 to aa244 of SEQ ID NO: 16, aa377 to aa379 of SEQ ID NO: 16, aa614 to aa616 of SEQ ID NO: 16, aa645 to aa647 of SEQ ID NO: 16, aa828 to aa830 of SEQ ID NO: 16, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(c) aa82 to aa84 of SEQ ID NO: 18, aa116 to aa118 of SEQ ID NO: 18, aa 122 to aa124 of SEQ ID NO: 18, aa239 to aa241 of SEQ ID NO: 18, aa374 to aa376 of SEQ ID NO: 18, aa611 to aa613 of SEQ ID NO: 18, aa642 to aa644 of SEQ ID NO: 18, aa825 to aa827 of SEQ ID NO: 18, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(d) aa83 to aa85 of SEQ ID NO: 26 or 28, aa117 to aa119 of SEQ ID NO: 26 or 28, aa123 to aa125 of SEQ ID NO: 26 or 28, aa 237 to aa239 of SEQ ID NO: 26 or 28, aa372 to aa374 of SEQ ID NO: 26 or 28, aa609 to aa 611 of SEQ ID NO: 26 or 28, aa640 to aa642 of SEQ ID NO: 26 or 28, aa823 to aa825 of SEQ ID NO: 26 or 28, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids; and

(e) aa62 to aa64 of SEQ ID NO: 30, aa96 to aa98 of SEQ ID NO: 30, aa102 to aa104 of SEQ ID NO: 30, aa216 to aa218 of SEQ ID NO: 30, aa351 to aa353 of SEQ ID NO: 30, aa588 to aa590 of SEQ ID NO: 30, aa619 to aa621 of SEQ ID NO: 30, or aa802 to aa804 of SEQ ID NO: 30, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids.

In some embodiments, the Ube3a polypeptide, protein or the biological equivalent thereof comprises one or more of mutated amino acid residue(s) at aa position(s) corresponding to one or more of those selected from the following, thereby forming one or more of glycosylation site(s):

aa64 of SEQ ID NO: 14, aa98 of SEQ ID NO: 14, aa104 of SEQ ID NO: 14, aa221 of SEQ ID NO: 14, aa356 of SEQ ID NO: 14, aa591 of SEQ ID NO: 14, aa624 of SEQ ID NO: 14, aa 805 of SEQ ID NO: 14, aa 806 of SEQ ID NO: 14, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa87 of SEQ ID NO: 16, aa121 of SEQ ID NO: 16, aa127 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, aa829 of SEQ ID NO: 16, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa84 of SEQ ID NO: 18, aa118 of SEQ ID NO: 18, aa124 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, aa826 of SEQ ID NO: 18, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa85 of SEQ ID NO: 26 or 28, aa119 of SEQ ID NO: 26 or 28, aa125 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, aa823 of SEQ ID NO: 26 or 28, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

aa64 of SEQ ID NO: 30, aa98 of SEQ ID NO: 30, aa104 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, or aa802 of SEQ ID NO: 30, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids.

In some embodiments, the Ube3a polypeptide, protein or the biological equivalent thereof comprises nine mutated amino acid residues at aa positions corresponding to those identified in any one of the following (a) to (e), thereby forming eight glycosylation sites:

(a) aa64 of SEQ ID NO: 14, aa98 of SEQ ID NO: 14, aa104 of SEQ ID NO: 14, aa221 of SEQ ID NO: 14, aa356 of SEQ ID NO: 14, aa591 of SEQ ID NO: 14, aa624 of SEQ ID NO: 14, aa 805 of SEQ ID NO: 14, and aa 806 of SEQ ID NO: 14, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(b) aa87 of SEQ ID NO: 16, aa121 of SEQ ID NO: 16, aa127 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, and aa829 of SEQ ID NO: 16, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(c) aa84 of SEQ ID NO: 18, aa118 of SEQ ID NO: 18, aa124 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, and aa826 of SEQ ID NO: 18, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(d) aa85 of SEQ ID NO: 26 or 28, aa119 of SEQ ID NO: 26 or 28, aa125 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, and aa823 of SEQ ID NO: 26 or 28, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids; or

(e) aa64 of SEQ ID NO: 30, aa98 of SEQ ID NO: 30, aa104 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, and aa802 of SEQ ID NO: 30, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids.

As illustrated in FIG. 16A, an online tool NetNGlyC was used to predict potential glycosylation sites. Four N-glycosylation sites were identified, among which only the site starting at amino acid 82 of SEQ ID NO: 8 is at a position corresponding to a glycosylation site created by the mutation as discussed in (a) to (c). Additionally, NetNGlyC does not reveal any mutations as disclosed herein.

In some embodiments, the Ube3a polypeptide, protein or the biological equivalent thereof comprises eight mutated amino acid residues at aa positions corresponding to those identified in any one of the following (a′) to (e′), thereby forming seven glycosylation sites:

(a′) aa64 of SEQ ID NO: 14, aa98 of SEQ ID NO: 14, aa221 of SEQ ID NO: 14, aa356 of SEQ ID NO: 14, aa591 of SEQ ID NO: 14, aa624 of SEQ ID NO: 14, aa 805 of SEQ ID NO: 14, and aa 806 of SEQ ID NO: 14, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(b′) aa87 of SEQ ID NO: 16, aa121 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, and aa829 of SEQ ID NO: 16, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(c′) aa84 of SEQ ID NO: 18, aa118 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, and aa826 of SEQ ID NO: 18, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids;

(d′) aa85 of SEQ ID NO: 26 or 28, aa119 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, and aa823 of SEQ ID NO: 26 or 28, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids; and

(e′) aa64 of SEQ ID NO: 30, aa98 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, and aa802 of SEQ ID NO: 30, or a position shifting any one of the identified position herein to the C terminus or the N terminus on the polypeptide, protein or the equivalent by about 1 amino acid, about 2 amino acids, about 3 amino acids, about 4 amino acids, about 5 amino acids, about 6 amino acids, about 7 amino acids, about 8 amino acids, about 9 amino acids, or about 10 amino acids.

In some embodiments, the formed glycosylation site comprises a consensus sequence of NXaaT or NXaaS, wherein Xaa is any amino acid residue optionally except proline (P).

In some embodiments, the mutated amino acid residue(s) is/are selected from one or more of:

Threonine (Thr or T) or Serine (Ser or S) at an aa position corresponding to aa64 of SEQ ID NO: 14, T or S at an aa position corresponding to aa98 of SEQ ID NO: 14, T or S at an aa position corresponding to aa104 of SEQ ID NO: 14, T or S at an aa position corresponding to aa221 of SEQ ID NO: 14, T or S at an aa position corresponding to aa356 of SEQ ID NO: 14, Asparagine (Asn or N) at an aa position corresponding to aa591 of SEQ ID NO: 14, T or S at an aa position corresponding to aa624 of SEQ ID NO: 14, N at an aa position corresponding to aa 805 of SEQ ID NO: 14, and N at an aa position corresponding to aa 806 of SEQ ID NO: 14;

T or S at an aa position corresponding to aa87 of SEQ ID NO: 16, T or S at an aa position corresponding to aa121 of SEQ ID NO: 16, T or S at an aa position corresponding to aa127 of SEQ ID NO: 16, T or S at an aa position corresponding to aa244 of SEQ ID NO: 16, T or S at an aa position corresponding to aa379 of SEQ ID NO: 16, N at an aa position corresponding to aa614 of SEQ ID NO: 16, T or S at an aa position corresponding to aa647 of SEQ ID NO: 16, N at an aa position corresponding to aa828 of SEQ ID NO: 16, and N at an aa position corresponding to aa829 of SEQ ID NO: 16;

T or S at an aa position corresponding to aa84 of SEQ ID NO: 18, T or S at an aa position corresponding to aa118 of SEQ ID NO: 18, T or S at an aa position corresponding to aa124 of SEQ ID NO: 18, T or S at an aa position corresponding to aa241 of SEQ ID NO: 18, T or S at an aa position corresponding to aa376 of SEQ ID NO: 18, N at an aa position corresponding to aa611 of SEQ ID NO: 18, T or S at an aa position corresponding to aa644 of SEQ ID NO: 18, N at an aa position corresponding to aa825 of SEQ ID NO: 18, and N at an aa position corresponding to aa826 of SEQ ID NO: 18;

T or S at an aa position corresponding to aa85 of SEQ ID NO: 26 or 28, T or S at an aa position corresponding to aa119 of SEQ ID NO: 26 or 28, T or S at an aa position corresponding to aa125 of SEQ ID NO: 26 or 28, T or S at an aa position corresponding to aa239 of SEQ ID NO: 26 or 28, T or S at an aa position corresponding to aa374 of SEQ ID NO: 26 or 28, N at an aa position corresponding to aa609 of SEQ ID NO: 26 or 28, N at an aa position corresponding to aa610 of SEQ ID NO: 26 or 28, T or S at an aa position corresponding to aa642 of SEQ ID NO: 26 or 28, and N at an aa position corresponding to aa823 of SEQ ID NO: 26 or 28; or

T or S at an aa position corresponding to aa64 of SEQ ID NO: 30, T or S at an aa position corresponding to aa98 of SEQ ID NO: 30, T or S at an aa position corresponding to aa104 of SEQ ID NO: 30, T or S at an aa position corresponding to aa218 of SEQ ID NO: 30, T or S at an aa position corresponding to aa353 of SEQ ID NO: 30, N at an aa position corresponding to aa588 of SEQ ID NO: 30, N at an aa position corresponding to aa589 of SEQ ID NO: 30, T or S at an aa position corresponding to aa621 of SEQ ID NO: 30, and N at an aa position corresponding to aa802 of SEQ ID NO: 30.

In some embodiments, the Ube3a polypeptide, protein or the biological equivalent thereof comprises an amino acid sequence selected from the following: aa21 to aa 872 of SEQ ID NO: 14, aa21 to aa 895 of SEQ ID NO: 16, aa21 to aa 892 of SEQ ID NO: 18, aa21 to aa 890 of SEQ ID NO: 26, aa21 to aa 890 of SEQ ID NO: 28, aa21 to aa 869 of SEQ ID NO: 30, or a sequence having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% identity to each thereof. In a further embodiment, the Ube3a polypeptide, protein or the biological equivalent thereof comprises one or more of glycosylation sites as disclosed herein.

In some embodiments, the Ube3a polypeptide, protein or the biological equivalent thereof further comprises a signal peptide. In a further embodiment, the signal peptide is a secretion signal peptide (which is also referred to herein as a secretion signal). In some embodiments, the signal peptide or secretion signal is selected from an antibody heavy/light chain secretion signal, a twin-arginine transport protein secretion signal, an Interleukin-2 (IL2) secretion signal, an Interleukin-4 (IL4) secretion signal, Interleukin-10 (IL10) secretion signal, an Interleukin-3 (IL3) secretion signal, an Interleukin-7 (IL7) secretion signal, an human IL2 secretion signal, a human OSM secretion signal, a VSV-G secretion signal, a Mouse Ig Kappa secretion signal, a Human IgG2 H secretion signal, a BM40 secretion signal, a Secrecon secretion signal, a Human IgKVIII secretion signal, a CD33 secretion signal, a tPA secretion signal, a Human Chymotrypsinogen secretion signal, a Human trypsinogen-2 secretion signal, a Gaussia luc secretion signal, a Albumin (HSA) secretion signal, an Influenza Haemagglutinin secretion signal, a Human insulin secretion signal, or a Silkworm Fibroin LC. In one embodiment, the signal peptide or the secretion signal comprises an amino acid sequence of aa1 to aa20 of SEQ ID NO:14. In some embodiments, the signal peptide or secretion signal is located at the N terminus of the Ube3a polypeptide, protein or the biological equivalent thereof. In some embodiments, the Ube3a polypeptide, protein or the biological equivalent thereof begins with a signal peptide or secretion signal on its N terminus.

In some embodiments, the Ube3a polypeptide, protein or the biological equivalent thereof comprises an amino acid sequence selected from any one of SEQ ID NOs: 14, 16, 18, 26, 28 and 30, or a sequence having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% identity to each thereof. In a further embodiment, the Ube3a polypeptide, protein or the biological equivalent thereof comprises one or more of glycosylation sites as disclosed herein.

In some embodiments, the Ube3a protein, polypeptide or a biological equivalent thereof is encoded by a polynucleotide as disclosed herein or equivalents thereof. In one aspect, the polynucleotide equivalents maintain at least one, or at least two, or at least three, or at least four, or at least five, or at least six, or at least seven, or at least eight of glycosylation sites in the encoded Ube3a protein, polypeptide or a biological equivalent thereof. In some embodiments, the encoded Ube3a protein, polypeptide or a biological equivalent thereof comprises eight or more glycosylation sites. In some embodiments, a polynucleotide as disclosed herein encodes a biological equivalent of the Ube3a protein or polypeptide having one or more non-naturally occurring glycosylation sites.

In some embodiments, the Ube3a protein, polypeptide or a biological equivalent thereof is encoded by a polynucleotide selected from any one or more of the following: nt 61 to nt 2619 of SEQ ID NO: 13, nt 61 to nt 2688 of SEQ ID NO: 15, nt 61 to nt 2679 of SEQ ID NO: 17, nt 61 to nt 2673 of SEQ ID NO: 25, nt 61 to nt 2673 of SEQ ID NO: 27, nt 61 to nt 2610 of SEQ ID NO: 29; SEQ ID NO: 13, SEQ ID NO: 15, SEQ ID NO: 17, SEQ ID NO: 25, SEQ ID NO: 27, SEQ ID NO: 29, or a sequence having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% identity to each thereof. In a further embodiment, the Ube3a polypeptide, protein or the biological equivalent thereof comprises one or more of glycosylation sites as disclosed herein.

In some embodiments, the recombinant and/or isolated polynucleotide comprises, or alternatively consists essentially of, or yet further consists of a sequence selected from any one or more of the following: nt 61 to nt 2619 of SEQ ID NO: 13, nt 61 to nt 2688 of SEQ ID NO: 15, nt 61 to nt 2679 of SEQ ID NO: 17, nt 61 to nt 2673 of SEQ ID NO: 25, nt 61 to nt 2673 of SEQ ID NO: 27, nt 61 to nt 2610 of SEQ ID NO: 29; SEQ ID NO: 13, SEQ ID NO: 15, SEQ ID NO: 17, SEQ ID NO: 25, SEQ ID NO: 27, SEQ ID NO: 29, or a sequence having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% identity to each thereof. In a further embodiment, the Ube3a polypeptide, protein or the biological equivalent thereof comprises one or more of glycosylation sites as disclosed herein.

In some embodiments, a mutated amino acid (aa) or nucleotide (nt) residue refers to the aa residue or nt residue is different from the residue at a corresponding position in a reference sequence. In some embodiments, a mutated protein, polypeptide, or polynucleotide comprises a mutated aa or nt residue. In some embodiments, the reference sequence is a naturally occurring and/or wildtype sequence. In one embodiments, the reference sequence is an amino acid sequence and comprises, or alternatively consists essentially of, or yet further consists of a sequence selected from one or more of SEQ ID NOs: 8, 10, 12, 20, 22, 24 or a natural variant of each thereof. In one embodiments, the reference sequence is a nucleotide sequence and comprises, or alternatively consists essentially of, or yet further consists of a sequence selected from one or more of SEQ ID NOs: 7, 9, 11, 19, 21, 23 or a natural variant of each thereof.

In some embodiments, the polynucleotide further comprises a regulatory sequence which direct expression of the Ube3a polypeptide, protein or the biological equivalent thereof. In some embodiments, wherein the regulatory sequence comprises one or more of the following: a promoter, an intron, an enhancer, a polyadenylation signal, a terminator, a silencer, a TATA box, or a Woodchuck Hepatitis Virus (WHP) Posttranscriptional Regulatory Element (WPRE). In a further embodiment, a polynucleotide as disclosed herein further comprises a promoter operatively linked to the polynucleotide for expression of the polynucleotide. Non-limiting examples of such include a promoter selected from a pol II promoter, e.g., an MNDU3 promoter, a CMV promoter, a PGK promoter, and an EF1alpha promoter. Sequences of these and other pol II promoters are known in the art. The sequence of the MNDU3 promoter and the sequence of an exemplary CMV promoter are provided herein. In one embodiment, the MNDU3 promoter comprises, or alternatively consists essentially of, or yet further consists of a sequence of SEQ ID NO: 3. In one embodiment, the CMV promoter comprises, or alternatively consists essentially of, or yet further consists of a sequence selected from SEQ ID NO: 1, 2, or 34. In one embodiment, the PKG promoter comprises, or alternatively consists essentially of, or yet further consists of a sequence of SEQ ID NO: 4. In one embodiment, the MNDU promoter comprises, or alternatively consists essentially of, or yet further consists of a sequence of SEQ ID NO: 5. In one embodiment, the EF1alpha promoter comprises, or alternatively consists essentially of, or yet further consists of a sequence of SEQ ID NO: 6. The polynucleotides can further comprise an enhancer element operatively linked to the polynucleotide encoding the mutated Ube3a protein, to increase or enhance expression of the polynucleotide.

In a further aspect, the polynucleotide further comprises a polynucleotide encoding a signal peptide and/or a secretion signal located 5′ to the polynucleotide encoding the modified Ube3a protein. Non-limiting examples of such signal peptide and/or secretion signal include a single chain fragment variable signal peptide, a twin-arginine transport protein signal peptide, an IL-4 secretion signal, an IL-2 secretion signal and an IL-10 secretion signal. An exemplary secretion IL-2 secretion signal polynucleotide is provided herein.

In some embodiments, further comprising one or more of the following: a polypurine tract sequence (PPT), a central PPT (cPPT), an R region, a U5, an encapsidation signal (Psi), a Rev-Responsive Element (RRE), a full-length U3 or a fragment thereof, a detectable or selection maker, a polynucleotide encoding a detectable or selection polypeptide, a regulatory sequence directing expression of the detectable or selection polypeptide, or a coding sequence for a cleavable peptide located between the coding sequence for the detectable or selection polypeptide and the sequence encoding the Ube3a polypeptide or protein or a biological equivalent thereof. In some embodiments, the cleavable peptide is a self-cleaving peptide, optionally a 2A self-cleaving peptide. In some embodiments, the 2A self-cleaving peptide is selected from P2A, T2A, E2A, F2A and BmCPV2A.

In some embodiments, the genetic information of the viral vector particle (which is also referred to herein as a vector genome or a viral genome) is RNA which comprises, or alternatively consists essentially of, or yet further consists of, on the 5′ and 3′ ends, the minimal LTR regions required for integration of the vector, and a polynucleotide as disclosed herein between the two LTR regions. In some embodiments, between the two LTR regions further comprises an encapsidation signal (a psi region) which is required for packaging of the vector RNA into the particle. In some embodiments, the psi region is followed by a Rev-Responsive Element (RRE) and a central polypurine tract sequence (cPPT) that enhance vector production by transporting the full-length vector transcript out of the nucleus for efficient packaging into the vector particle.

Further provided are polynucleotides which are equivalents, complements, reverse sequences, or reverse complements to the modified Ube3a coding polynucleotides. In some embodiments, an equivalent nucleic acid, polynucleotide or oligonucleotide is one having at least 70% sequence identity, or alternatively at least 75% sequence identity, or alternatively at least 80% sequence identity, or alternatively at least 85% sequence identity, or alternatively at least 90% sequence identity, or alternatively at least 91% sequence identity, or alternatively at least 92% sequence identity, or alternatively at least 93% sequence identity, or alternatively at least 94% sequence identity, or alternatively at least 95% sequence identity, or alternatively at least 96% sequence identity, or alternatively at least 97% sequence identity, or alternatively at least 98% sequence identity, or alternatively at least 99% sequence identity to the reference nucleic acid, polynucleotide, or oligonucleotide. Additionally or alternatively, an equivalent nucleic acid, polynucleotide or oligonucleotide hybridizes under conditions of high stringency to any one of the reference polynucleotide, its complement, or its reverse complement.

Additionally or alternatively, the equivalent nucleic acid, polynucleotide or oligonucleotide must encode a functional Ube3a protein, polypeptide or a biological equivalent thereof that optionally can be identified through one or more assays described herein. In some embodiments, an equivalent nucleic acid, polynucleotide, or oligonucleotide has at least 70% sequence identity, or alternatively at least 75% sequence identity, or alternatively at least 80% sequence identity, or alternatively at least 85% sequence identity, or alternatively at least 90% sequence identity, or alternatively at least 91% sequence identity, or alternatively at least 92% sequence identity, or alternatively at least 93% sequence identity, or alternatively at least 94% sequence identity, or alternatively at least 95% sequence identity, or alternatively at least 96% sequence identity, or alternatively at least 97% sequence identity, or alternatively at least 98% sequence identity, or alternatively at least 99% sequence identity to the reference nucleic acid, polynucleotide, or oligonucleotide. Additionally or alternatively, an equivalent nucleic acid, polynucleotide or oligonucleotide hybridizes under conditions of high stringency to any one of the reference polynucleotide, its complement, or its reverse complement.

Some embodiments are with the proviso that one or more polynucleotides identified herein having one or more glycosylation sites are mutated from a polynucleotide selected from any one of SEQ ID NOs: 7, 9, 11, 19, 21, or 23. Some embodiments are with the proviso that one or more polynucleotides identified herein having one or more glycosylation sites are not mutated from a polynucleotide selected from any one of SEQ ID NOs: 7, 9, 11, 19, 21, or 23, but from a natural variant of each thereof. Some embodiments are with the proviso that one or more polynucleotides identified herein having one or more glycosylation sites further comprises one or more mutation(s) which do not forming a glycosylation site.

The polynucleotides can further comprise a polynucleotide that is, or encodes a detectable or purification marker.

Further provided are the recombinant and/or isolated polypeptides encoded by the polynucleotides and equivalents of each thereof. In some embodiment, an equivalent or biological equivalent protein or polypeptide is one having at least 70% sequence identity, or alternatively at least 75% sequence identity, or alternatively at least 80% sequence identity, or alternatively at least 85% sequence identity, or alternatively at least 90% sequence identity, or alternatively at least 91% sequence identity, or alternatively at least 92% sequence identity, or alternatively at least 93% sequence identity, or alternatively at least 94% sequence identity, or alternatively at least 95% sequence identity, or alternatively at least 96% sequence identity, or alternatively at least 97% sequence identity, or alternatively at least 98% sequence identity, or alternatively at least 99% sequence identity to a reference protein or polypeptide and/or a polypeptide or protein as disclosed herein (such as any one of SEQ ID NOs: 8, 10, 12, 20, 22, or 24, or a polypeptide or protein encoded by an equivalent polynucleotide as noted herein).

In some embodiments, the equivalent or biological equivalent protein or polypeptide is a functional protein that optionally can be identified through one or more assays described herein. In some embodiments, the equivalent or biological equivalent protein or polypeptide has at least 70% sequence identity, or alternatively at least 75% sequence identity, or alternatively at least 80% sequence identity, or alternatively at least 85% sequence identity, or alternatively at least 90% sequence identity, or alternatively at least 91% sequence identity, or alternatively at least 92% sequence identity, or alternatively at least 93% sequence identity, or alternatively at least 94% sequence identity, or alternatively at least 95% sequence identity, or alternatively at least 96% sequence identity, or alternatively at least 97% sequence identity, or alternatively at least 98% sequence identity, or alternatively at least 99% sequence identity to a reference protein or polypeptide and/or a polypeptide or protein as disclosed herein (such as any one of SEQ ID NOs: 8, 10, 12, 20, 22, or 24, or a polypeptide or protein encoded by an equivalent polynucleotide as noted herein).

Some embodiments are with the proviso that one or more amino acid residues identified herein as mutated to form a possible glycosylation site in a Ube3a protein, polypeptide or a biological equivalent thereof are mutated from a polypeptide selected from any one of SEQ ID NOs: 8, 10, 12, 20, 22, or 24. Some embodiments are with the proviso that one or more amino acid residues identified herein as mutated to form a possible glycosylation site in a Ube3a protein, polypeptide or a biological equivalent thereof are not mutated from a polypeptide selected from any one of SEQ ID NOs: 8, 10, 12, 20, 22, or 24, but from a natural variant of each thereof. Some embodiments are with the proviso that one or more amino acid residues identified herein as mutated to form a possible glycosylation site in a Ube3a protein, polypeptide or a biological equivalent thereof further comprises one or more mutation(s) which do not forming a glycosylation site. Some embodiments are with the proviso that one or more amino acid residues identified herein as mutated to form a possible glycosylation site in a Ube3a protein, polypeptide or a biological equivalent thereof are not mutated from a polypeptide selected from a non-natural variant of any one of SEQ ID NOs: 8, 10, 12, 20, 22, or 24. In a further embodiment, such non-natural variant is a Ube3a biological equivalent of the corresponding SEQ ID NOs: 8, 10, 12, 20, 22, or 24

The polypeptides can further comprise a detectable or a purification marker. The polypeptides and protein can be expressed in any appropriate system, e.g., a prokaryotic or eukaryotic system, such as for example a mammalian or human cell.

### Vectors

This disclosure also provides a vector comprising, or alternatively consisting essentially of, or yet further consisting of a polynucleotide as disclosed herein, optionally inserted into a viral backbone. In some embodiments, the vector is selected for expression in prokaryotic or eukaryotic cells. In some embodiments, the vector comprises, or alternatively consists essentially of, or yet further consists of a polynucleotide as described herein, encoding the modified protein. In some embodiments, the vector comprises, or alternatively consists essentially of, or yet further consists of a polynucleotide as described herein, permitting replication of the polynucleotide (which is also referred to herein as a modified gene). In further embodiments, the vector further comprises a regulatory sequence operatively linked to the modified gene and directing the replication of the modified gene. In yet a further embodiment, the regulatory sequence comprises, or alternatively consists essentially of, or yet further consists of one or more of: a promoter, an intron, an enhancer, a polyadenylation signal, a terminator, a silencer, a TATA box, or a Woodchuck Hepatitis Virus (WHP) Posttranscriptional Regulatory Element (WPRE).

In some embodiments, the vector is a non-viral vector, optionally a plasmid. In some embodiments, the vector is a viral vector, optionally selected from a retroviral vector (such as a lentiviral vector), an adenoviral vector, an adeno-associated viral vector, or a Herpes viral vector. In a further embodiment, the viral backbone contains essential nucleic acids or sequences for integration of the modified gene into a target cell's genome. In some embodiments, the essential nucleic acids necessary for integration to the genome of the target cell include at the 5′ and 3′ ends the minimal LTR regions required for integration of the vector.

In some embodiments, the term “vector” intends a recombinant vector that retains the ability to infect and transduce non-dividing and/or slowly-dividing cells and integrate into the target cell's genome. In several embodiments, the vector is derived from or based on a wild-type virus. In further embodiments, the vector is derived from or based on a wild-type adenovirus, adeno-associated virus, or a retrovirus such as a lentivirus. Examples of retrovirus include without limitation, human immunodeficiency virus (HIV), equine infectious anaemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference. In some embodiments, the lentiviral vector is a self-inactivating lentiviral vector. In further embodiments, the lentiviral vector has a U3 region lacking a TATA box. Additionally or alternatively, the lentiviral vector has a U3 region lacking one or more of transcription factor binding site(s).

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome, the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Thus, in one aspect, provided is a vector comprising a recombinant polynucleotide as disclosed herein encoding a Ube3a protein, polypeptide or a biological fragment thereof having one or more glycosylation sites for use in gene therapy and research. In some embodiments, the Ube3a protein is naturally occurring. In some embodiments, it is recombinantly produced by modification of one or more nucleotides that modify an amino acid. In some embodiments, the Ube3a protein, polypeptide or a biological fragment thereof has three or more glycosylation sites. In some embodiments, the Ube3a protein, polypeptide or a biological fragment thereof has four or more glycosylation sites. In some embodiments, the protein, polypeptide or a biological fragment thereof is encoded by the polynucleotide shown in the Sequence Listing and equivalents of each thereof. In some embodiments, the equivalents maintain at least one or more the identified glycosylation sites. In some embodiments, the Ube3a protein, polypeptide or a biological fragment thereof has eight or more glycosylation sites. In further embodiments, the polynucleotide further comprises a nucleotide sequence encoding a cell penetrating domain located downstream of the signal sequence.

In some embodiments, the vector comprises a polynucleotide and a promoter operatively linked to the polynucleotide. Non-limiting examples of such promoters include pol II promoters optionally selected from the group of an MNDU3 promoter, a minimal cytomegalovirus promoter (CMV) promoter, a phosphoglycerate kinase promoter (PKG) promoter, and an EF1alpha promoter. In some embodiments, the vector further comprises a polynucleotide encoding a secretion signal located 5′ to the polynucleotide encoding the modified Ube3a protein. Non-limiting examples of such secretion signals include a single chain fragment variable secretion signal, a twin-arginine transport protein secretion signal, an IL-4 secretion signal, an IL-2 secretion signal and an IL-10 secretion signal. An exemplary secretion signal polynucleotide include, but are not limited to, those shown in the Sequence Listing provided below and equivalents thereof. The vector can further comprise a polynucleotide that is, or encodes a detectable or purification marker. Alternative polymerase II promoters include, but are not limited to LTRs from retroviral and lentiviral vectors.

In some embodiments, the polynucleotide and/or vector further comprises a marker or detectable label such as a gene encoding an enhanced green fluorescent protein (EGFP), red fluorescence protein (RFP), green fluorescent protein (GFP) and yellow fluorescent protein (YFP) or the like. These are commercially available and described in the technical art. They can be expressed from the same or separate regulatory sequences, such as promoter, driving the expression of the modified Ube3a protein. In some embodiments, the promoter is a PGK promoter.

In some embodiments, the vector comprises a sequence encoding a cell penetrating domain, which for example, can comprise, or alternatively consist essentially of, or yet further consist of a human immunodeficiency virus transactivator of transcription (HIV-TAT) peptide.

A CPP as employed in accordance with one aspect of the disclosure may include 3 to 35 amino acids, preferably 5 to 25 amino acids, more preferably 10 to 25 amino acids, or even more preferably 15 to 25 amino acids.

A CPP suitable for carrying out one aspect of the disclosure may include at least one basic amino acid such as arginine, lysine and histidine. In some embodiments, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular embodiment, the CPP includes at least two, three, four, or five consecutive arginine. In a further embodiment, the CPP includes more arginine than lysine or histidine, or preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

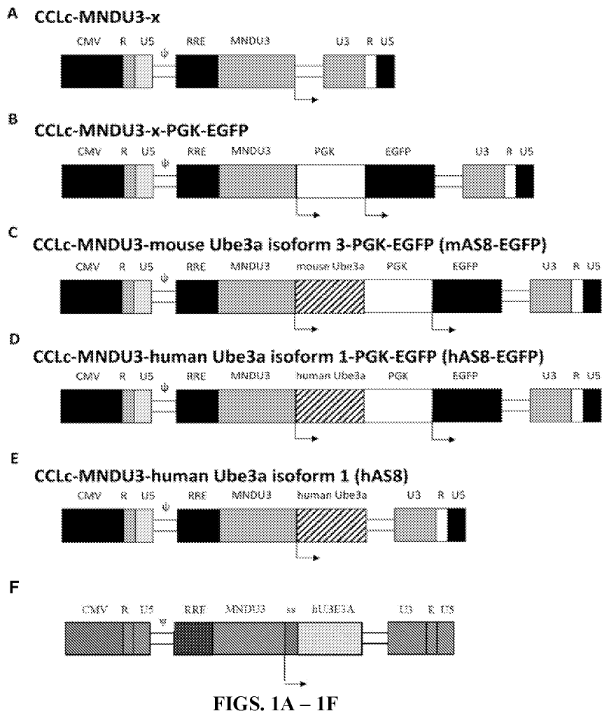

In some embodiments, provided is a vector as illustrated in any one of FIG. 1A to FIG. 1F with or without a detectable label or purification marker. In some embodiments, the vector comprises a sequence of SEQ ID NO: 35 and further comprises a polynucleotide sequence as disclosed herein inserted after the MNDU3 promoter in the sequence of SEQ ID NO: 35.

In some embodiments, provided is a vector (such as a retroviral vector and/or a lentiviral vector) produced by those illustrated in any one of FIG. 1A to FIG. 1F. In further embodiments, the retroviral and/or lentiviral vector comprises a polynucleotide (such as a RNA) encoded by any vector as illustrated in any one of FIG. 1A to FIG. 1F. In yet further embodiments, the retroviral and/or lentiviral vector comprises a polynucleotide (such as a RNA) encoded by a vector comprising a sequence of SEQ ID NO: 35 and further comprising a polynucleotide (such as a RNA) encoding a Ube3a protein, polypeptide or a biological equivalent thereof, or a reverse complement of the Ube3a coding polynucleotide inserted after the MNDU3 promoter in the sequence of SEQ ID NO: 35.

In some embodiments, provided is a vector (such as a retroviral vector and/or a lentiviral vector) comprising a polynucleotide (such as a RNA) encoding a Ube3a protein, polypeptide or a biological equivalent thereof, or a reverse complement of the polynucleotide. In some embodiments, a polynucleotide of the vector (such as a retroviral vector and/or a lentiviral vector) comprises, or alternatively consists essentially of, or yet further consists of one or more of the following: (1) an R region, (2) a U5, (3) a Psi, (4) an RRE, (5) a promoter (such as an MNDU3 promoter), (6) a polynucleotide (such as a RNA) encoding a Ube3a protein, polypeptide or a biological equivalent thereof, or a reverse complement of the polynucleotide, (7) a U3, (8) an R region, and (9) a U5, optionally from 5′ to 3′.

Further comprising are the polypeptides encoded by these polynucleotides, vectors and host cell systems.

### Packaging Systems

The disclosure also provides a viral packaging system comprising: a vector as described herein, optionally wherein the backbone is derived from a virus; a packaging plasmid; and an envelope plasmid. The packaging plasmid contains polynucleotides encoding the nucleoside, matrix proteins, capsids, and other components necessary for packaging a vector genome into a viral particle. Packaging plasmids are described in the patent literature, e.g., U.S. Pat. Nos. 7,262,049; 6,995,258; 7,252,991 and 5,710,037, incorporated herein by reference.

The system may also contain a plasmid encoding a pseudotyped envelope protein provided by an envelope plasmid. Pseudotyped viral vectors consist of vector particles bearing glycoproteins derived from other enveloped viruses or alternatively containing functional portions. See, for example U.S. Pat. No. 7,262,049, incorporated herein by reference. In some embodiments, the envelope plasmid encodes an envelope protein optionally not causing the viral particle to non-specifically bind to a cell or population of cells. The specificity of the viral particle may be conferred by a protein or polypeptide, such as an antibody binding domain, that is inserted into the particle envelope. Examples of suitable envelope proteins include, but are not limited to those containing the VSVG or RD114 domains.

This disclosure also provides the suitable packaging cell line. In one aspect, the packaging cell line is the HEK-293 cell line. Other suitable cell lines are known in the art, for example, described in the patent literature within U.S. Pat. Nos. 7,070,994; 6,995,919; 6,475,786; 6,372,502; 6,365,150 and 5,591,624, each incorporated herein by reference.

### Viral Particles and Methods to Produce the Viral Particles

This disclosure further provides a method for producing a viral particle comprising the Ube3a protein, polynucleotide, or a biological equivalent thereof comprising, or alternatively consisting essentially of, or yet further consisting of, transducing a packaging cell line with a viral system as described above, under conditions suitable to package the viral vector. Such conditions are known in the art and briefly described herein. The viral particle can be isolated from the cell supernatant, using methods known to those of skill in the art, e.g., centrifugation. Such isolated particles are further provided by this disclosure.

This disclosure further provides the isolated viral particle produced by this method. The viral particle comprises, or alternatively consists essentially of, or yet further consists of a polynucleotide as disclosed herein.

This disclosure also provides methods to prepare a viral particle comprising a polynucleotide as disclosed herein, such as the modified Ube3a gene as disclosed herein by transducing a packaging cell line, as described herein with the vector, the envelope plasmid and the packaging plasmid under conditions that facilitate packaging of the vector into the envelope particle. In some embodiments, the viral particle is a pseudotyped viral particle. In further embodiments, the particles are separated from the cellular supernatant and conjugated to an antibody for cell-specific targeting.

In some embodiments, the genetic information of the viral vector particle (which is also referred to herein as a vector genome or a viral genome) is RNA which comprises, or alternatively consists essentially of, or yet further consists of, on the 5′ and 3′ ends, the minimal LTR regions required for integration of the vector, and a polynucleotide as disclosed herein between the two LTR regions. In some embodiments, between the two LTR regions further comprises an encapsidation signal (a psi region) which is required for packaging of the vector RNA into the particle. In some embodiments, the psi region is followed by a Rev-Responsive Element (RRE) and a central polypurine tract sequence (cPPT) that enhance vector production by transporting the full-length vector transcript out of the nucleus for efficient packaging into the vector particle.

In some embodiments, the vector further comprises a polymerase-II promoter, such as MNDU3, which drives the expression of the modified Ube3a gene. In some embodiments, the vector comprises a marker, e.g., an EGFP gene (enhanced Green Fluorescent Protein) optionally which is driven by a polymerase II promoter, such as a PGK promoter. The EGFP gene is used as a reporter gene to detect transduced cells.

In some embodiments, the listed genetic elements are transcribed into a full-length RNA molecule which is packaged into a vector particle and contains all of the genetic information that will be integrated into the transduced cells.

In some embodiments, the full-length RNA transcript is packaged inside the capsid of the vector particle that contains the nucleocapsid, capsid, and matrix proteins which are generated from the packaging plasmid such as delta-8.91. In some embodiments, the reverse transcriptase polymerase which is generated from the packaging plasmid delta-8.91 is also located within the capsid with the RNA transcript. In some embodiments, the capsid encases and protects the full-length RNA transcript.

In some embodiments, cells of a packaging cell line, such as HEK-293T cells are plated at 75% confluence in complete DMEM media 24 hours prior to transfection. After at least 24 hours post-plating of cells, the transfection mixture is prepared. Three milliliters of serum free media are incubated with 150 ul of the lipofection reagent for 20 minutes at room temperature. The plasmids are then added to the media/lipofection reagent mixture at a ratio of 5:5:2 (packaging plasmid: viral vector plasmid: envelope plasmid) and incubated for 30 minutes. After this final incubation period, the media/lipofection reagent/DNA mixture is then added to the HEK-293T cells and left overnight for the transfection to occur. The next day, the transfection media is removed and fresh complete DMEM is added. Seventy-two hours later, the cell culture supernatant can be collected and concentrated by ultracentrifugation at 20,000 rpm for 1.5 hours.

Once the vector particle buds from the packaging cells and is released into the supernatant, this vector particle can be isolated and/or purified by an antibody specifically recognizes or binds the particle and/or by having a conjugated antibody on the envelope of the particle as defined herein.

### Cells and Cell Populations

Provided herein is a cell comprising one or more of the following: a recombinant polynucleotide as disclosed herein, a vector as disclosed herein, a recombinant Ube3a protein, polypeptide or the biological equivalent thereof as disclosed herein, thereby producing the polynucleotide, the vector, or the recombinant Ube3a protein, polypeptide or biological equivalent thereof. In some embodiments, the cell is an isolated cell and/or an engineered cell. In some embodiments, the cell is a eukaryotic or a prokaryotic cell. In some embodiments, the cell is a mammalian cell. In some embodiments, the cell is an in vitro and/or ex vivo cell. In some embodiments, the cell is an in vivo cell in a subject.

Also provided is a clonal population of a cell as disclosed herein.

Additional provided is a method to express a secreted Ube3a protein, polypeptide or the biological equivalent thereof comprising growing a cell as disclosed herein under conditions that allow for the expression of the recombinant Ube3a protein or polypeptide or a biological equivalent thereof.

Yet further provided is a cell or population of cells, comprising, or alternatively consisting essentially of, or yet further consisting of, one or more of: a polynucleotide as disclosed herein, a vector as disclosed herein, and a Ube3a protein, polypeptide or a biological equivalent thereof as disclosed herein. In some embodiments, the vector is a viral particle. In some embodiment, the cell or population of cells further comprises a detectable marker.

In some embodiments, the cell is an isolated cell. In some embodiments, the cell is not a naturally occurring cell. In some embodiments, the cell is also referred to herein as a host cell. In some embodiments, the isolated host cell is a packaging cell line. In some embodiments, the cell is a eukaryotic cell, such as a mammalian cell. In further embodiments, the cell is a murine cell or a human cell.

Additionally or alternatively, the cell is a progenitor cell or a progeny thereof. In some embodiments, the cell is a stem cell, e.g., an embryonic stem cell, an induced pluripotent stem cell (iPSC), an adult stem cell, a mesenchymal stem cell, a neural stem cell, a hematopoietic stem cell (HSC), or a progeny of each thereof. In some embodiments, the vector and/or host cell can further comprise a detectable or purification label.

In some embodiments, the cell is a stem cell, such as a hematopoietic progenitor cell or a hematopoietic stem cell, e.g., a CD34+ cell. Alternatively, the stem cell is a neural stem cell or an iPSC.

In some embodiments, the cell is an immune cell, optionally selected from a B-cell, T-cell, Nature Killer (NK) cell, dendritic cell, a cell of the myeloid lineage, a neutrophil, a monocyte, a macrophage, and/or a microglia. In further embodiments, the immune cell is derived from a progenitor cell (such as a hematopoietic progenitor cell), a stem cell (e.g., an embryonic stem cell, an induced pluripotent stem cell (iPSC), an adult stem cell, a mesenchymal stem cell, a neural stem cell, a hematopoietic stem cell (HSC)), or a progeny of each thereof. In some embodiments, the T cell expresses CD4, i.e. is a CD4+ T cell. In some embodiments, the T cell expresses CD8, i.e. is a CD8+ T cell.

When used therapeutically, the cells can be allogeneic or autologous to the subject to be treated. The subjects can be mammalian, e.g., murine, canine, bovine, equine, ovine, feline or a human subject or patient.

In some embodiments, the cell expresses and/or secrets a recombinant Ube3a protein or polypeptide or a biological equivalent thereof as disclosed herein.

Also provided is a population of cells as disclosed herein and/or a progeny thereof.

This disclosure further provides an isolated cell or an enriched population of cells, optionally, that are derived or differentiated from the stem cell described above. In some instances, the derived or differentiated cell or the enriched population of cells comprise, or consist essentially of, or yet further consist of an immune cell. In some instances, the immune cell is selected from a B-cell, T-cell, Nature Killer (NK) cell, dendritic cell, a cell of the myeloid lineage, and/or a neutrophil. In some embodiments, the T cell expresses CD4, i.e. is a CD4+ T cell. In some embodiments, the T cell expresses CD8, i.e. is a CD8+ T cell. In some instances, the isolated cell or an enriched population of immune cells comprise, or consist essentially of, or yet further consist of, a monocyte, a macrophage, and/or a microglia. In some cases, one or more types of the immune cells described herein are modified with a recombinant polynucleotide encoding an Ube3a protein described herein to generate an Ube3a expressing immune cell. In some cases, a B-cell, a T-cell, an NK cell, a dendritic cell, a neutrophil, or a cell of the myeloid lineage is modified (for example, is transduced or transfected) with a recombinant polynucleotide encoding a Ube3a protein described herein to generate a modified cell expressing an Ube3a protein, polypeptide or a biological equivalent thereof. In some cases, a macrophage is modified (for example, is transduced or transfected) with a recombinant polynucleotide encoding a modified Ube3a protein described herein to generate an Ube3a expressing macrophage in vivo and/or in vitro. In some cases, a CD34+ HSC is modified (for example, is transduced or transfected) with a recombinant polynucleotide encoding a modified Ube3a protein described herein to generate an Ube3a expressing HSC and/or macrophage in vivo and/or in vitro.

In some embodiments, the cell population expresses CD4, CD14 and HLADR. In some embodiments, at least about 60%, or at least about 70%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the cells in the population are CD4+, i.e. expressing CD4 optionally on the cell surface. Additionally or alternatively, at least about 60%, or at least about 70%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the cell in the population are CD14+, i.e., expressing CD14 optionally on the cell surface. Additionally or alternatively, at least about 60%, or at least about 70%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the cells in the population are HL-ADR+, i.e., expressing HLA-DR optionally on the cell surface.

In some embodiments, the cell population derives macrophages under a suitable condition, see, the Experimental Methods for an example.

In some embodiments, the cell population comprises substantially macrophages, optionally derived from a stem cell such as a HSC. In some embodiments, the cell population comprises substantially a stem cell, such as HSCs, optionally deriving to macrophages.

In some embodiments, the cell population is substantially homogenous, for example, at least about 60%, or at least about 70%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99% of the cells in the population are the same.

These cells are useful to treat and/or prevent Angelman syndrome in a subject in need thereof or to test new therapies. The subject can be a fetus, an infant, a juvenile or an adult.

### Compositions, Screens and Therapeutic Uses

Provided by this disclosure is a composition comprising, or alternatively consisting essentially of, or yet further consisting of any one or more of: a polynucleotide as disclosed herein, a Ube3a protein, polypeptide or a biological equivalent thereof as disclosed herein, a vector as disclosed herein, a cell as disclosed herein, a cell population as disclosed herein, a clonal population as disclosed herein and/or a packaging system as disclosed herein and a carrier. In some embodiments, the carrier is a pharmaceutically acceptable carrier.

Also provided by this disclosure is a kit comprising, or alternatively consisting essentially of, or yet further consisting of an optional instruction for use and any one or more of: a probe for detecting a defective Ube3a gene, a polynucleotide as disclosed herein, a Ube3a protein, polypeptide or a biological equivalent thereof as disclosed herein, a vector as disclosed herein, a cell as disclosed herein, a cell population as disclosed herein, a clonal population as disclosed herein, a packaging system as disclosed herein, and/or a composition as disclosed herein. In some embodiments, the instruction is for use in a method as disclosed herein.

These compositions and/or kits can be used diagnostically or therapeutically as described herein. Additionally or alternatively, these compositions can be used in combination with other known therapies.

The compositions can be used in vitro to screen for small molecules and other agents that may modify the effectiveness of the therapy alone or in combination with other therapies by adding to the composition varying amounts of the agent to be tested and comparing it to a companion system that does not have the agent but which exhibits the desired therapeutic effect optionally achieved by one or more of: a polynucleotide as disclosed herein, a Ube3a protein, polypeptide or a biological equivalent thereof as disclosed herein, a vector as disclosed herein, a cell as disclosed herein, a cell population as disclosed herein, and/or a composition as disclosed herein.

When the polynucleotides, vectors, polypeptides, cells, and/or compositions are administered to an appropriate animal subject, the animal subject can be used as an animal model to test alternative therapies in the same manner as the in vitro screen.

Also provided is a method to express a Ube3a protein, polypeptide or a biological equivalent thereof, comprising, or alternatively consisting essentially of, or yet further consisting of, growing the host cell as described herein under conditions that allow for the expression of the Ube3a protein, polypeptide or a biological equivalent thereof. The method can be practiced in vitro, ex vivo or in vivo. In some embodiments, the expressed Ube3a protein, polypeptide or a biological equivalent thereof is secreted, optionally out of the cell expressing such protein, polypeptide or biological equivalent thereof.

Further provided is a method to express a Ube3a protein, polypeptide or a biological equivalent thereof in a subject, comprising or alternatively consisting essentially of, or yet further consisting of, administering, for example, an effective amount of, one or more of: a polynucleotide as disclosed herein, a vector as disclosed herein, and/or a cell as described herein, to the subject, thereby expressing Ube3a in the subject. In some embodiments, the polynucleotide encodes a Ube3a protein, polypeptide or a biological equivalent thereof, wherein the protein, polypeptide or biological equivalent thereof has one or more glycosylation sites. In further embodiments, the one or more glycosylation sites are non-naturally occurring.

In some embodiments, the expressed Ube3a protein, polypeptide or a biological equivalent thereof is secreted out of the cell producing such protein, polypeptide or biological equivalent thereof. In some embodiments, the expressed Ube3a protein, polypeptide or a biological equivalent thereof is secreted to blood of the subject. In further embodiments, the expressed Ube3a protein, polypeptide or a biological equivalent thereof is secreted to peripheral blood of the subject. Additionally or alternatively, the expressed Ube3a protein, polypeptide or a biological equivalent thereof is secreted to the subject brain within the blood-brain barrier. In some embodiments, the expressed Ube3a protein, polypeptide or a biological equivalent thereof binds to a neuron cell, and optionally enters the neuron cell.

In some embodiments, the subject is a mammal, e.g., a human patient. In some embodiments, the subject is deficient or carries a defective Ube3a gene. In some embodiments, the subject is asymptomatic for Angelman syndrome or Prader-Willi syndrome, or symptomatic for these syndromes. In some embodiments, the subject is a fetus, an infant or a pre-pubescent subject. In some embodiments, the subject is an adult.

Further provided is a method to treat, prevent, halt or reverse Angelman syndrome in a subject carrying a defective Ube3a gene or allele, comprising, or alternatively consisting essentially of, or yet further consisting of, administering, for example an effective amount of, one or more of: a polynucleotide as disclosed herein, a vector as disclosed herein, a Ube3a protein, polypeptide, or a biological equivalent thereof as disclosed herein, a cell as described herein, a cell population as disclosed herein, and/or a composition as disclosed herein, to the subject, thereby expressing the Ube3a protein, polypeptide, or biological equivalent thereof and/or delivering the Ube3a protein, polypeptide or a biological equivalent thereof in brain and/or to a neuron of the subject and/or treating Angelman syndrome and/or Prader-Willi syndrome.

In some embodiments, the subject is deficient or carries a defective Ube3A gene. In some embodiments, the subject is a mammal, e.g. a human patient. In some embodiments, the subject is asymptomatic for Angelman syndrome. In some embodiments, the subject is a fetus, an infant or a pre-pubescent subject. In some embodiments, the subject is an adult.

Also provided is a method for enhanced delivery of a Ube3a protein, polypeptide or a biological equivalent thereof in brain and/or to a neuron comprising, or alternatively consisting essentially of, or yet further consisting of, administering, for example, an effective amount of, one or more of: a polynucleotide as disclosed herein, a vector as disclosed herein, a Ube3a protein, polypeptide, or a biological equivalent thereof as disclosed herein, a cell as described herein, a cell population as disclosed herein, and/or a composition as disclosed herein, to the subject, thereby expressing the Ube3a protein, polypeptide, or biological equivalent thereof and/or delivering the Ube3a protein, polypeptide or a biological equivalent thereof in brain and/or to a neuron of the subject and/or treating Angelman syndrome.

In some embodiments relating to any methods, compositions or others disclosed herein, the subject is deficient or carries a defective Ube3A gene. In further embodiments, the subject comprises and/or expresses a defective Ube3A protein. In one embodiment, the defective Ube3A protein is not a biological equivalent of a Ube3A protein. In one embodiments, the defective Ube3A protein performs a Ube3A function, such as ubiquitinating S5a or another protein, at a level of less than about 50%, less than about 40%, less than about 30%, less than about 20%, less than about 10%, less than about 5%, less than about 3%, less than about 2%, less than about 1%, or less than about 0.1% of a wildtype one. In yet further embodiments, the subject comprises and/or expresses Ube3A protein or a biological equivalent thereof at a decreased level compared to a healthy control. In one embodiment, the healthy control is a subject free of any disease. In another embodiment, the healthy control is a subject free of a disease as disclosed herein. In yet another embodiment, the healthy control is a subject free of AS. In one embodiments, the subject comprises and/or expresses Ube3A protein or a biological equivalent thereof at a level of less than about 50%, less than about 40%, less than about 30%, less than about 20%, less than about 10%, less than about 5%, less than about 3%, less than about 2%, less than about 1%, or less than about 0.1% of a healthy control. In some embodiments, the subject is a mammal, e.g. a human patient. In some embodiments, the subject is asymptomatic for Angelman syndrome. In some embodiments, the subject is symptomatic for Angelman syndrome. In some embodiments, the subject is a fetus, an infant or a pre-pubescent subject. In some embodiments, the subject is an adult.

Additional effective therapies can be combined with this disclosure and/or added as necessary.

In some embodiments, an “effective amount” is delivered, that is it is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the gene or protein that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to provide therapeutic benefit.

The term administration shall include without limitation, local or systemic administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository), intracranial, or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule. In some embodiments, the administration is performed locally, such as to the bone marrow or in the brain. In some embodiments, the administration is performed systemically. In some embodiments, the administration is an infusion, for example over about 1 hour, about 1.5 hours, about 2 hours, about 3 hours, about 4 hours, about 5 hours, about 6 hours, about 7 hours, about 8 hours, about 12 hours, or about 1 day.

In some embodiments, the cell or cell population is administered at a dose of about 1.0×10⁴ to about 1×10¹⁵ cells/kg body weight of the subject.

In some embodiments, the dose is at least about 1×10⁴ CD34+ cells/kg, or at least about 2×10⁴ CD34+ cells/kg, or at least about 3×10⁴ CD34+ cells/kg, or at least about 4×10⁴ CD34+ cells/kg, or at least about 5×10⁴ CD34+ cells/kg, or at least about 6×10⁴ CD34+ cells/kg, or at least about 7×10⁴ CD34+ cells/kg, or at least about 8×10⁴ CD34+ cells/kg, or at least about 9×10⁴ CD34+ cells/kg, or at least about 1×10⁵ CD34+ cells/kg, or at least about 2×10⁵ CD34+ cells/kg, or at least about 3×10⁵ CD34+ cells/kg, or at least about 4×10⁵ CD34+ cells/kg, or at least about 5×10⁵ CD34+ cells/kg, or at least about 6×10⁵ CD34+ cells/kg, or at least about 7×10⁵ CD34+ cells/kg, or at least about 8×10⁵ CD34+ cells/kg, or at least about 9×10⁵ CD34+ cells/kg, or at least about 1×10⁶ CD34+ cells/kg, or at least about 2×10⁶ CD34+ cells/kg, or at least about 3×10⁶ CD34+ cells/kg, or at least about 4×10⁶ CD34+ cells/kg, or at least about 5×10⁶ CD34+ cells/kg, or at least about 6×10⁶ CD34+ cells/kg, or at least about 7×10⁶ CD34+ cells/kg, or at least about 8×10⁶ CD34+ cells/kg, or at least about 9×10⁶ CD34+ cells/kg, or at least about 1×10⁷ CD34+ cells/kg, or at least about 2×10⁷ CD34+ cells/kg, or at least about 3×10⁷ CD34+ cells/kg, or at least about 4×10⁷ CD34+ cells/kg, or at least about 5×10⁷ CD34+ cells/kg, or at least about 6×10⁷ CD34+ cells/kg, or at least about 7×10⁷ CD34+ cells/kg, or at least about 8×10⁷ CD34+ cells/kg, or at least about 9×10⁷ CD34+ cells/kg, or at least about 1×10⁸ CD34+ cells/kg, or at least about 2×10⁸ CD34+ cells/kg, or at least about 3×10⁸ CD34+ cells/kg, or at least about 4×10⁸ CD34+ cells/kg, or at least about 5×10⁸ CD34+ cells/kg, or at least about 6×10⁸ CD34+ cells/kg, or at least about 7×10⁸ CD34+ cells/kg, or at least about 8×10⁸ CD34+ cells/kg, or at least about 9×10⁸ CD34+ cells/kg, or at least about 1×10⁹ CD34+ cells/kg body weight of the subject.

Additionally or alternatively, the dose is less than 1×10⁹, or less than 9×10⁸, or less than 8×10⁸, or less than 7×10⁸, or less than 6×10⁸, or less than 5×10⁸, or less than 4×10⁸, or less than 3×10⁸, or less than 2×10⁸, or less than 1×10⁸, or less than 9×10⁷, or less than 8×10⁷, or less than 7×10⁷, or less than 6×10⁷, or less than 5×10⁷, or less than 4×10⁷, or less than 3×10⁷, or less than 2×10⁷, or less than 1×10⁷, or less than 9×10⁶, or less than 8×10⁶, or less than 7×10⁶, or less than 6×10⁶, or less than 5×10⁶, or less than 4×10⁶, or less than 3×10⁶, or less than 2×10⁶, or less than 1×10⁶, or less than 9×10⁵, or less than 8×10⁵, or less than 7×10⁵, or less than 6×10⁵, or less than 5×10⁵, or less than 4×10⁵, or less than 3×10⁵, or less than 2×10⁵, or less than 1×10⁵ cells, such as CD34+ HSC/kg body weight of the subject.

Additionally or alternatively, the dose is less than 1×10⁹, or less than 9×10⁸, or less than 8×10⁸, or less than 7×10⁸, or less than 6×10⁸, or less than 5×10⁸, or less than 4×10⁸, or less than 3×10⁸, or less than 2×10⁸, or less than 1×10⁸, or less than 9×10⁷, or less than 8×10⁷, or less than 7×10⁷, or less than 6×10⁷, or less than 5×10⁷, or less than 4×10⁷, or less than 3×10⁷, or less than 2×10⁷, or less than 1×10⁷, or less than 9×10⁶, or less than 8×10⁶, or less than 7×10⁶, or less than 6×10⁶, or less than 5×10⁶, or less than 4×10⁶, or less than 3×10⁶, or less than 2×10⁶, or less than 1×10⁶, or less than 9×10⁵, or less than 8×10⁵, or less than 7×10⁵, or less than 6×10⁵, or less than 5×10⁵, or less than 4×10⁵, or less than 3×10⁵, or less than 2×10⁵, or less than 1×10⁵ modified cells such as modified CD34+ cells/kg body weight of the subject.

The following examples are intended to illustrate, and not limit the embodiments disclosed herein.

### Experimental Methods

### Experiment No. 1—Vector Construction

Lentiviral Vector Design and Production

For the purpose of the following studies in this experiment, all lentiviral vectors were generated using a self-inactivating third-generation lentiviral vector backbone CCLc-x (FIG. 1A). For in vivo efficacy studies, a lentiviral vector expressing a modified form of mouse Ube3a isoform 3 was used since these experiments were performed in the B6-IL2−/− Ube3a−/+ mouse model. To generate the mouse Ube3a expressing lentiviral vectors, a modified form of mouse isoform 3 (modifications include adding an N-terminal secretion signal and eight N-glycosylation sites throughout the protein) was synthesized and cloned into the CCLc-x vector backbone under the control of an MNDU3 promoter (FIG. 1C). An EGFP gene under the control of a PGK promoter was cloned downstream to track transduction and engraftment of transduced cells. Wild type Ube3a is an intracellular protein and does not get secreted from the cell. Therefore, a secretion signal was added so that it can be secreted from the transduced cells. N-glycosylation sites are used for binding to the mannose-6-phosphate receptor found on neurons. These sites were added to the mouse Ube3a protein to allow for efficient attachment and uptake into neurons after secretion from the transduced cells. For the in vivo experiments, a lentiviral vector expressing a modified form of human Ube3a isoform 1 was used. Human isoform 1 is species-equivalent to mouse isoform 3. The human Ube3a isoform 1 gene contains the same N-terminal secretion signal and the same eight N-glycosylation site modifications as the mouse Ube3a isoform 3 gene used in the in vivo efficacy studies. The modified human Ube3a isoform 1 gene was synthesized and cloned into the CCLc-x vector under the control of an MNDU3 promoter (FIG. 1E and FIG. 1F). An EGFP gene under the control of a PGK promoter was cloned downstream from the human Ube3a gene to be used for tracking during the safety/toxicity experiments (FIG. 1D) but is omitted for clinical use. A control empty vector containing only the EGFP reporter gene was generated by cloning the EGFP gene under the control of the PGK promoter (FIG. 1B). Experiments involving recombinant DNA were performed following the NIH guidelines.

Lentiviral vectors were generated using GMP-equivalent reagents in human embryonic kidney (HEK)-293 cells by transfecting the cells with a 1:5:5 ratio of envelope vesicular stomatitis virus glycoprotein (VSVG), a packaging plasmid (48.9) containing the vector capsid and reverse transcriptase genes, and one of the above mentioned transfer plasmids, either one of the Ube3a vectors or the control EGFP vector. Forty-eight hours post-transfection, vector supernatants were collected and concentrated by ultrafiltration. Transducing unit titers of each vector were calculated by transducing HEK-293 cells and analyzing EGFP expression by flow cytometry forty-eight hours post-transduction for vectors containing the EGFP expression cassette. For the Ube3a vectors that do not contain EGFP, total genomic DNA from the transduced cells was extracted and analyzed by quantitative PCR using Taqman Real-Time PCR Master Mix with a vector specific psi primer and probe set.

Ube3a Vector Functionality-Overexpression and Ubiquitination Activity of Lentivector Expressed Ube3a in Human CD34+ HPC Derived Macrophages

To evaluate the expression and functionality of the human Ube3a lentivector, human CD34+ HPSC were transduced with the hAS8 vector and derived, in vitro, into mature macrophages. Human CD34+ HSPC were isolated from umbilical cord blood obtained from the UC Davis Umbilical Cord Blood Collection Program by Ficoll-Paque density gradient, and further purified by CD34 magnetic bead column separation. Total CD34+ cells were cultured for forty-eight hours in XVIVO-10 media supplemented with 50 ng/ml stem cell factor (SCF), thrombopoietin (TPO), and Flt-3 ligand. After forty-eight hours post-isolation, the CD34+ cells were left either nontransduced (NT) or transduced with either the EGFP control vector or the hAS8 vector at an MOI of 20 with 8 mg/ml protamine sulfate for a minimum of 3 hours at 37 degrees Celsius. EGFP control and hAS8 vector transduced CD34+ cells were then sorted based on EGFP expression for subsequent experiments.

Colony Forming Unit Assay (CFU)

Due to the overexpression of HexA and HexB in CD34+ HSPC by lentiviral transduction, it is possible that detrimental growth and differentiation of the cells could occur. To evaluate this, an HSPC CFU assay was performed. CD34+ cells, either NT, fluorescently activated cell sorted (FACS) EGFP control vector transduced, or FACS sorted hAS8 vector transduced cells (500 total cells) were cultured for 12 days in methylcellulose medium supplemented with cytokines. Following the culture time period, the total burst forming unit-erythroid colonies (BFU-E), granulocyte/macrophage (GM) colonies, and granulocyte/erythrocyte/megakaryocyte/macrophage (GEMM) colonies were observed by microscopy and counted. Experiments were performed in triplicate.

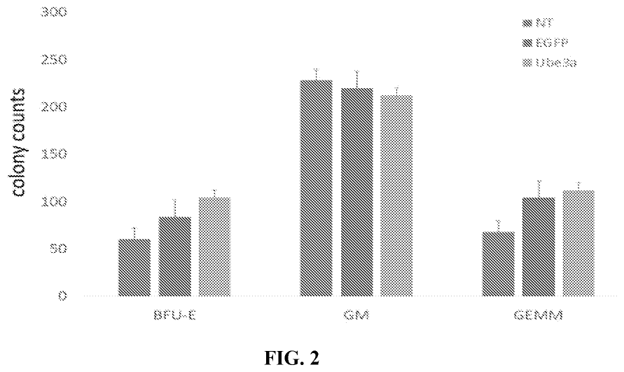

As displayed in FIG. 2, similar levels of the three colony types were observed in the Ube3a lentivector transduced cells compared to the control cells.

Derivation of Phenotypically Normal Macrophages

Cells from the CFU assay were further differentiated into mature macrophages in vitro. CFUs derived from the NT and vector transduced CD34+ cells were further derived into mature macrophages by plating the cells in six-well plates with DMEM supplemented with 10% FBS, 10 ng/ml macrophage colony stimulating factor (M-CSF) and 10 ng/ml of granulocyte-macrophage colony stimulating factor (GM-CSF) for 4 days with media changes every 2 days. Cells were observed by microscopy to identify macrophage morphology and analyzed by flow cytometry for expression of normal macrophage cell surface markers. Macrophages were stained with phycoerythrin (PE)-conjugated CD14, PE-conjugated HLA-DR, or PE-conjugated CD4. Flow cytometry was performed using a Beckman Coulter Cytomics FC500 and CXP software. Experiments were performed in triplicate.

The macrophages were analyzed by flow cytometry with antibodies specific to the normal macrophage markers, CD4, CD14, and HLA-DR. Macrophages derived from CD34+ HPC transduced with the Ube3a lentivector were phenotypically normal displaying, on average, a CD4% of 96.1%, a CD14% of 99.6%, and an HLA-DR % of 99.1%. These levels were similar to those of the control NT and EGFP-alone macrophages which displayed CD4% levels of 95.7% and 96.4%, CD14% levels of 99.3% and 97.6%, and HLADR % levels of 98.0% and 97.5%, respectively.

Western Blots

Total cell extracts were then collected from the macrophages using a Pierce RIPA buffer supplemented with Halt Protease Inhibitor and flash frozen. Total protein concentrations were determined using a BCA Protein Assay Kit following the manufacturer's protocol. Proteins were loaded into polyacrylamide gels and transferred onto polyvinylidene fluoride membranes. Membranes were then incubated with their respective primary antibodies: mouse anti-human Ube3a. Goat anti-mouse horseradish peroxidase (HRP) conjugated secondary antibodies were then added. The blots were then developed using SuperSignal West Pico Chemiluminescent Substrate.

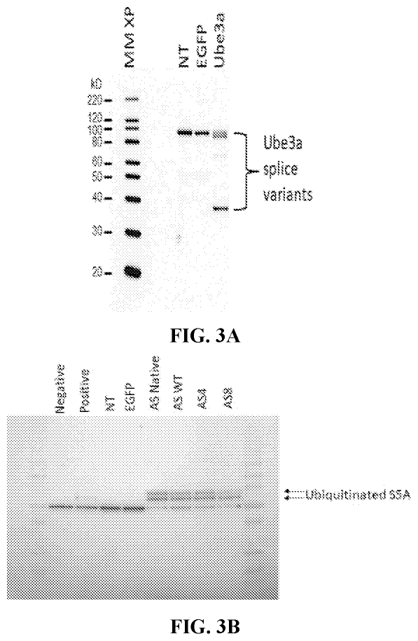

As displayed in FIG. 3A, overexpression of Ube3a was detected from the presence of a strong Ube3a band and its splice variants. This was in comparison to control nontransduced (NT) and control EGFP-alone (EGFP) vector transduced cells. To further evaluate whether the human Ube3a protein expressed by the lentivector was functional in ubiquitinating its target protein S5a, a ubiquitination assay (for example, (R&D Systems, Human E6AP/S5a ubiquitination kit, cat #K-230) was performed on human CD34+ HPC derived macrophage cell extracts, for example using method described in Yi et al. (2017) J. Biol. Chem. 28; 292(30):12503-12515. As displayed in FIG. 3B, ubiquitination of the S5a protein was detected in Ube3a vector transduced cells. This is displayed as a laddered banding pattern in the “AS8” lane.

Taken together, these results demonstrated the functionality of the human Ube3a lentiviral vector in overexpression of Ube3a and its subsequent ubiquitination of a target protein. This data also demonstrated normal human CD34+ HSPC colony formation and differentiation of phenotypically normal end stage macrophages, in vitro, upon transduction with the human Ube3a lentiviral vector.

### Experiment No. 2—Neonatal Treatment in Mouse Models

A novel immunodeficient Ube3a−/+ mouse model (BGU) capable of accepting human CD34+ cells for engraftment by crossing the Ube3a−/+ mice with the B6-IL2rg−/− knockout mice was generated. The BGU mice retain the phenotypes of normal immunocompetent Ube3a−/+ mice displaying signs of deficient motor, behavioral, and cognitive phenotypes, and a lower threshold of seizure induction. This mouse model was made as a preclinical evaluation of the human CD34+ cells transduced with a Ube3a expressing lentiviral vector.

Applicant demonstrated successful functionality, efficacy, and safety of a lentiviral vector expressing the Ube3a gene in human CD34+ HSC in humanized AS and NRG mouse models. Restoration of functional enzyme activity was observed in disease-specific cells and HSC-derived immune cells. Significant improvements in motor and behavioral phenotypes were observed in AS mice transplanted with the Ube3a vector transduced human HSC. Long term safety of the Ube3a lentiviral vector transduced human CD34+ HSC was also observed upon engraftment and multi-lineage hematopoiesis in a humanized NRG mouse model.

Functional Efficacy of HSC Following Neonatal Treatment with HSC Transduced with the Ube3a Lentiviral Vector

Applicant generated a humanized immunodeficient Ube3a-deficient mouse model (BGUbe3a) by crossing the Ube3a-deficient mice with immunodeficient B6-IL2rg−/− knockout mice. Transplanted human CD34+ hematopoietic stem cells (HSCs) can, therefore, successfully reconstitute the mouse with a human immune system and allowed the evaluation of the therapeutic candidate to be used clinically: human CD34+ HSC transduced with the Ube3a lentiviral vector. The BGUbe3a-deficient mice display AS-related phenotypes including motor, behavioral, and neurological deficits. They also display a wider gait and slower movements as compared to WT mice. For all experiments below, the clinically equivalent Ube3a vector (as displayed in FIG. 1B), was used to transduce the human CD34+ HSC used for the cell transplants. All in vivo efficacy data presented below are from mice transplanted intrahepatically at ages 2-5 days old. At this age, mice do not display symptoms of AS. The hypothesis of these experiments was to transplant mice and prevent AS-related phenotypes from occurring.

Functional Rescue of AS Phenotypes in Newborn Transplanted BGU Mice

To evaluate the ability of the Ube3a lentiviral vectors to improve AS phenotypes, mAS8 vector transduced human CD34+ HSC were transplanted into newborn immunodeficient BGU mice. Nontransduced or mAS8 (Ube3a) vector transduced human CD34+ HSC (500,000 cells) were transplanted intrahepatically into 2-5 day old BGU pups sublethally irradiated with 100 rad. Eight weeks post-transplant, mice were bled via tail vein and analyzed for engraftment by flow cytometry using a mouse anti-human CD45 antibody. Successfully engrafted mice were then evaluated for AS phenotypes. The order and age of testing were as follows: (1) open field at 9 weeks of age, (2) beam walking at 9 weeks of age, (3) rotarod at 10 weeks of age, (4) DigiGait at 10 weeks of age, and (5) novel object recognition at 11 weeks of age.

Testing Cohorts

Four genotype/treatment groups were analyzed for functional rescue in behavioral assays relevant to AS. The groups were control wildtype (WT; which has an IL2 null mutation for appropriate control of the immune system effect), HET (which are the novel AS model with a maternal deletion of Ube3a and created with an IL2 null mutation), NT-HET (which are the novel AS model with a maternal deletion of Ube3a, created with an IL2 null mutation and transplanted with nontransduced human CD34+ cells to control for the effect of HSC alone), and Ube3a-HET (which are the novel AS model with a maternal deletion of Ube3a, created with an IL2 null mutation and transplanted with human CD34+ HSC transduced with the Ube3a lentiviral vector). Sexes were combined since there has never been a sex difference in Angelman Syndrome preclinical or clinical outcomes.

Open Field Activity of BGUbe3a Mice Transplanted with Ube3a Vector Transduced Cells

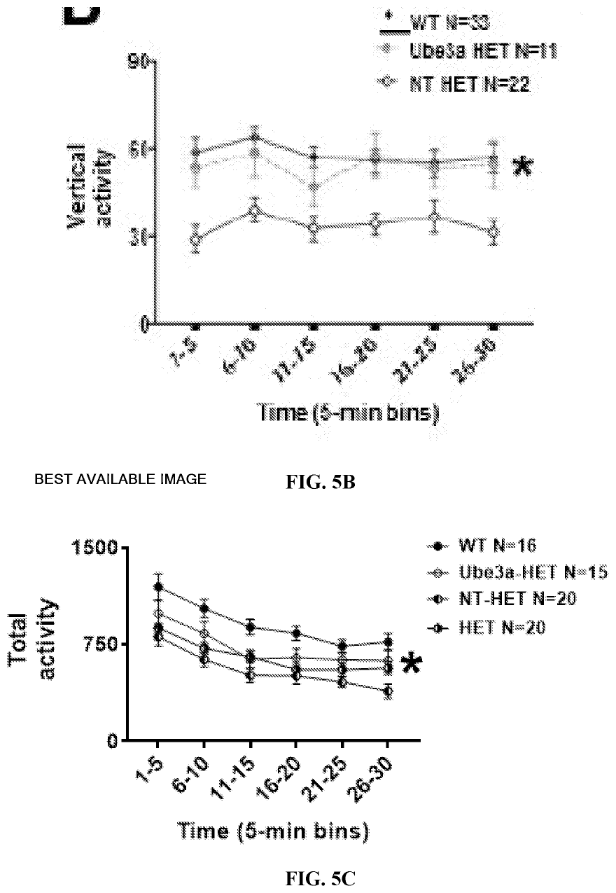

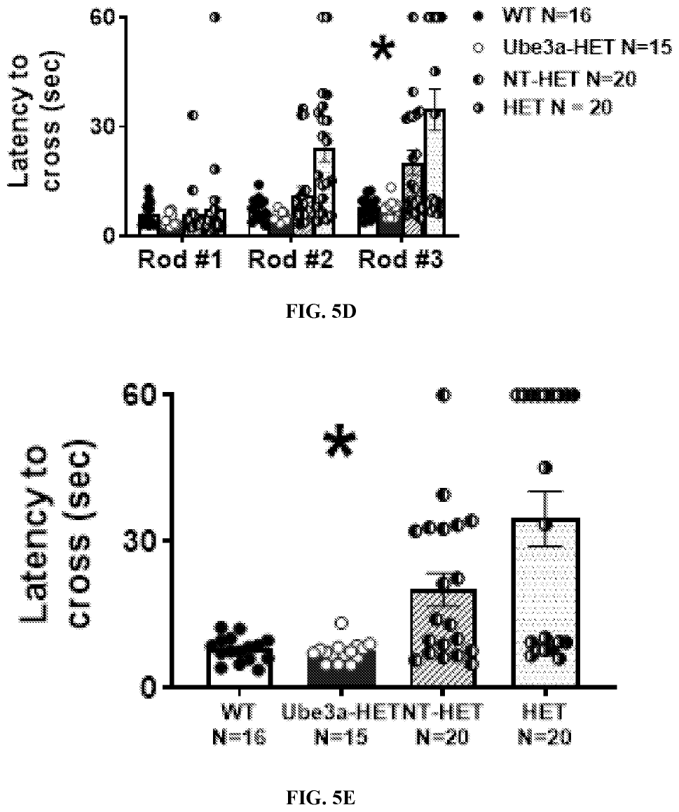

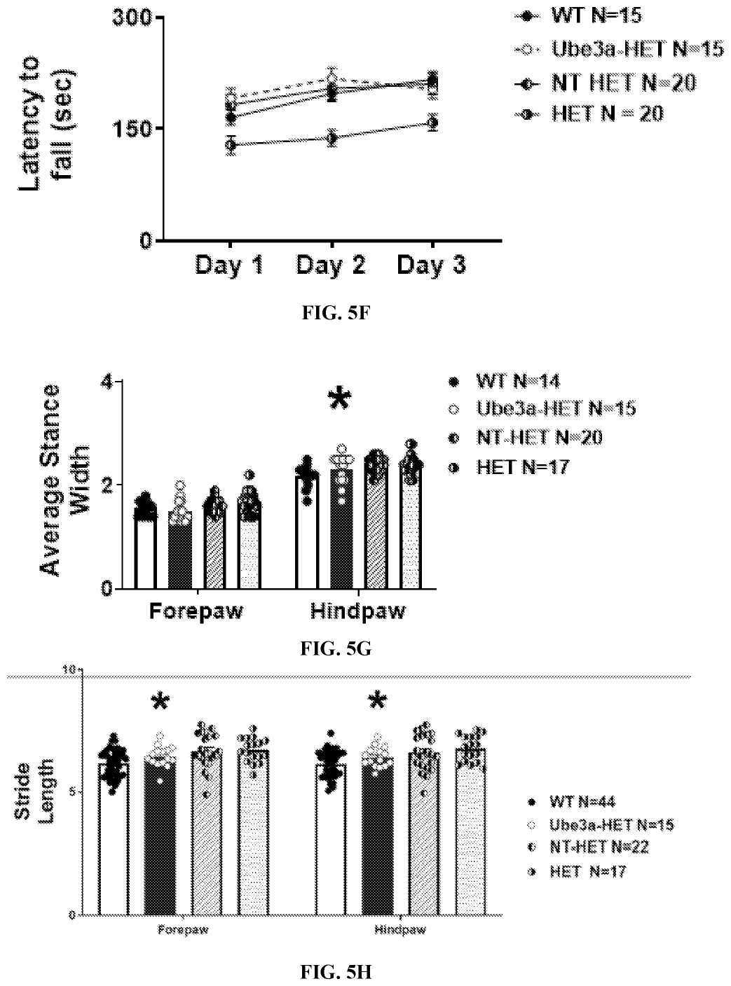

Ube3a-deficient mice display motor and behavioral deficits in an open field assay with decreased movements and total activity. Therefore, to evaluate whether human CD34+ HSC transduced with the Ube3a lentiviral vector prevented these deficits from occurring, mice were evaluated for horizontal, vertical, and total activity. Briefly, as displayed in FIGS. 5A-5C, Ube3a-deficient mice transplanted with the Ube3a vector transduced cells (Ube3a Het) performed significantly (p<0.0001) better than Ube3a-deficient mice transplanted with nontransduced human C34+ cells (NT Het) in (FIG. 5A) horizontal, (FIG. 5B) vertical, and (FIG. 5C) total activity. Ube3a Het mice performed similar to wild type (WT) mice. More details are discussed below.

General exploratory locomotion in a novel open field arena was evaluated as previously described (1-8). Briefly, each subject was tested in a VersaMax Animal Activity Monitoring System for 30 minutes in a ˜30 lux testing room. Total distance traversed, horizontal activity, vertical activity, and time spent in the center were automatically measured to assess gross motor abilities in mice.

Newborn BGU mice that were transplanted with Ube3a vector transduced (Ube3a-Het) human CD34+ HSC behaviorally tested eight-weeks post-transplant performed indistinguishable from WT on multiple assays of motoric behavioral deficits. Locomotion in a novel open field was collected assessing the total distance traversed and the horizontal movements using beam breaks in a novel arena.

Group differences in total distance were observed using a multi-factor repeated measures ANOVA (FIG. 5C; F (3, 67)=8.194, p<0.0001). Holm-Sidak corrected posthoc analysis for multiple comparisons highlighted that both NT-HET (p<0.0001) and HET (p<0.0028) differed from WT while the treatment group Ube3a-HET did not differ from WT (p>0.05). Holm-Sidak corrected posthoc analysis for multiple comparisons highlighted that both NT-HET (p<0.0001) and HET (p<0.0002) differed from WT while the treatment group Ube3a-HET did not differ from WT (p=0.191). The rigor of this posthoc analysis is remarkable. The Ube3a-treated HET do not differ from WT at any timepoint across the 30 minute assay (0-5 min, p=0.389, 6-10 min, p=0.5027, 11-15 min, p=0.132, 16-20 min, p=0.5418, 21-25 min p=0.995, 26-30 min p=0.8991), while the HET and WT, adjusted p's from Holm Sidak provided (0-5 min, p<0.0052, 6-10 min, p<0.0076, 11-15 min, p<0.004, 16-20 min, p<0.024, 21-25 min, 26-30 min) differed in 4 of the 6 5-min time bins and the NT-HET and WT differ on every 5 minute bin of the 30 minute task (0-5 min, p<0.0002, 6-10 min, p<0.000, 11-15 min, p<0.0004, 16-20 min, p<0.003, 21-25 min, p<0.0287, 26-30 min, p<0.0003).

In corroboration, the horizontal activity counts also illustrated group differences using a multi-factor repeated measures ANOVA (FIG. 5A; F (3, 67)=9.487, p<0.0001). Holm-Sidak corrected posthoc analysis for multiple comparisons highlighted that both NT-HET (p<0.0001) and HET (p<0.0002) differed from WT while the treatment group Ube3a-HET did not differ from WT (p=0.191). The rigor of this posthoc analysis is remarkable. The Ube3a-treated HET do not differ from WT at any timepoint across the 30 minute assay (0-5 min, p=0.4383, 6-10 min, p=0.3297, 11-15 min, p=0.0439, 16-20 min, p=0.2038, 21-25 min p=0.7833, 26-30 min p=2903), while the HET and WT, adjusted p's from Holm Sidak provided (0-5 min, p<0.0001, 6-10 min, p<0.0001, 11-15 min, p<0.0023, 16-20 min, p<0.0002, 21-25 min, p<0.0231, 26-30 min, p<0.0214) differed in 5 of the 6 5-min time bins and the NT-HET and WT differ on every 5 minute bin of the 30 minute task (0-5 min, p<0.0002, 6-10 min, p<0.0001, 11-15 min, p<0.0001, 16-20 min, p<0.0003, 21-25 min, p<0.0103, 26-30 min, p<0.0001).

Beam Walking Activity of Ube3a Mice Transplanted with Ube3a Vector Transduced Cells

To further evaluate the ability of the Ube3a vector transduced cells to prevent AS-related phenotypes, transplanted mice were subjected to a beam walking assay measured by latency time to cross three beams of varying width. Briefly, as displayed in FIG. 5D and FIG. 5E Ube3a-deficient mice transplanted with the Ube3a vector transduced human CD34+ HSC (Ube3a Het) displayed significantly (p<0.05) better performance in the beam walking activity compared to Ube3a-deficient mice transplanted with nontransduced (NT Het) cells. Ube3a Het mice performed similar to WT mice. More details are discussed below.

A beam walking motor task was conducted as previously described (1-8). Fifty-nine centimeter long round rods were suspended sixty-eight centimeters above a cushioned landing pad. A goal box at the end of the beam consisted of a twelve centimeter diameter cylinder to provide motivation to cross the beam. Each mouse was placed at one end of the beam and the time to cross to the goal box on the other end was measured. Testing sequence moved from largest diameter to smallest diameter rods in order of increased difficulty. On the day prior to testing, all animals were given two practice trials on the largest diameter round beam in order to become accustom to the procedure. On the test day, each animal was sequentially tested on three round rods (35, 18, and 13 mm). Testing sequence was based on presentations of decreasing diameter to present increasing levels of difficulty. Each mouse was given two trials on each beam, separated by approximately 30 minutes. The time to transverse the beam was recorded and averaged across the two trials for each beam. A maximum time of 60 seconds was assigned to individuals that failed to cross the beam in that duration. In the small number of cases where mice fell from the beam, a score of 60 seconds was assigned.

A balance beam walking motor task was conducted. All groups showed longer latencies to cross the rod shaped beams as they became thinner and more difficult to traverse, as expected. Group differences using a multi-factor repeated measures ANOVA (FIG. 5D; F (3, 67)=17.02, p<0.0001). Interestingly, Ube3a-deficient mice transplanted with the Ube3a lentivector transduced human CD34+ HSC (Ube3a-Het) exhibited wildtype values of performance on the beam, illustrated via Holm-Sidak posthoc analysis. The Ube3a-treated HET do not differ from WT on any of the rod #3 (rod #3, p>0.999) while the HET (p<0.0001) and NT-HET (p<0.227) had much slower latencies to cross rod #3, highlighting the improved motor coordination of the treated group. FIG. 5E illustrates the fastest groups to cross the rods, highlighting WT and Ube3a-HET, as being indistinguishable from one another by time across rod, highlighting substantial improvement in motor coordination.

Rotarod and Digigait Activity of Ube3a Mice Transplanted with Ube3a Vector Transduced Cells

As another test to evaluate the ability of the Ube3a vector transduced cells to prevent AS-related phenotypes, transplanted mice were subjected to rotarod and DigiGait assays. Rotarod assays determine latency to fall off a rod that slowly increases acceleration of a rotating rod. As displayed in FIG. 5F, Ube3a-deficient mice transplanted with the Ube3a vector transduced human CD34+ HSC (Ube3a Het) displayed significantly (p<0.05) better performance in the rotarod assay compared to Ube3a-deficient mice transplanted with nontransduced (NT Het) cells. Ube3a Het mice performed similar to WT mice. The DigiGait assay measures the width of the gaits of tested mice. As displayed in FIG. 5G, Ube3a-deficient mice transplanted with the Ube3a vector transduced human CD34+ HSC (Ube3a Het) displayed a significantly (p<0.05) improved gait in both forepaws and hind paws compared to Ube3a-deficient mice transplanted with nontransduced (NT Het) cells. The gaits of Ube3a Het mice looked similar to the gaits of WT mice. More details are discussed below.

Rotarod: Motor coordination, balance, and motor learning were tested with an accelerating rotarod from Ugo Basile as previously described (1-8). Mice were placed on a rotating cylinder that slowly accelerated from 5 to 40 revolutions per min over five minutes. Mice were given three trials per day with a 60 minute inter-trial rest interval and tested for 3 consecutive days for a total of nine trials. Performance was scored as latency to fall off the cylinder with a maximum latency of 5 minutes.

DigiGait: The DigiGait analyzer from Mouse Specifics Inc was used to analyze gait. DigiGait is a treadmill with a ventral plane camera positioned below a motorized transparent belt. Mice were habituated in the walking corridor for one minute prior to starting the belt for capturing images. The belt speed was set at 20 cm/s and each subject's paws were recorded for 5 seconds. Nine hundred video frames across the five second videos were collected at 180 frames per second. The captured frames were digitized, and relevant gait parameters were analyzed by DigiGait analysis software. Left and right fore and hind limbs were averaged together per subject. Animals unable to walk at the target speed for five seconds were allowed to rest and were retested. If they were unable to complete this criterion after three times, the subject was excluded from the study.

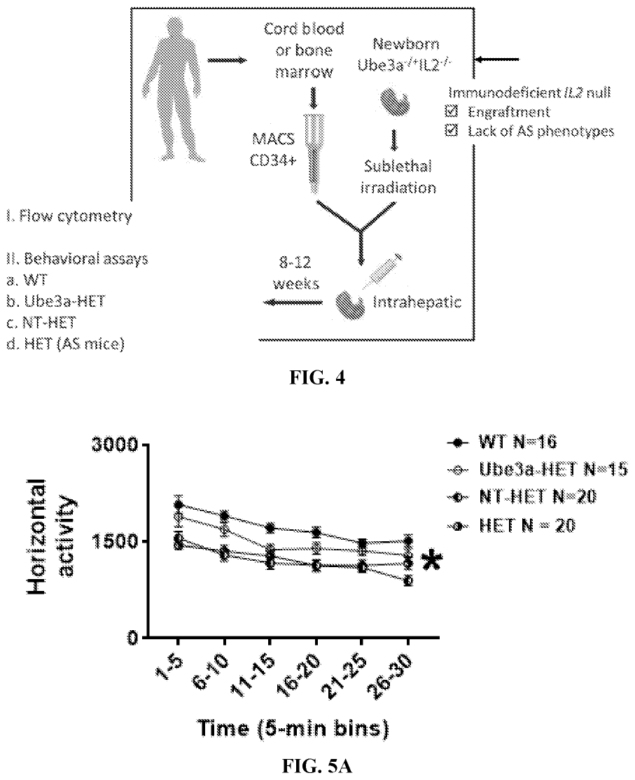

A secondary corroborating assay of motor coordination is the rotarod assay as mice have dramatic deficits in this task. As expected, HET differ from WT in their latencies to fall from the accelerating rod (FIG. 5F; F (3, 67)=8.395, p<0.0001) on days 2 (p<0.0098) and 3 (p<0.0133), illustrating poor motor coordination and a lack of motor learning. Strikingly, Ube3a-deficient mice transplanted with the Ube3a lentivector transduced human CD34+ HSC were similar to wildtype across all three days of testing using Holm-Sidak posthoc analyses (Day 1; p=0.8356), (Day 2; p=0.9572), and (Day 3; p=9979). Group differences were detected using a multi-factor ANOVA (FIG. 4F; F (3, 65)=5.782, p<0.002).

In multiple reports, AS patients, exhibit wide stances on the Zenowalk Way. (1-8) Digigait analysis showed HET (p<0.0026) and NT-HET (p<0.002) differ from wildtype while the Ube3a-treated HET group showed a narrowing of these wide stances (p=0.3486).

Novel Object Recognition (NOR) of Ube3a Mice Transplanted with Ube3a Vector Transduced Cells

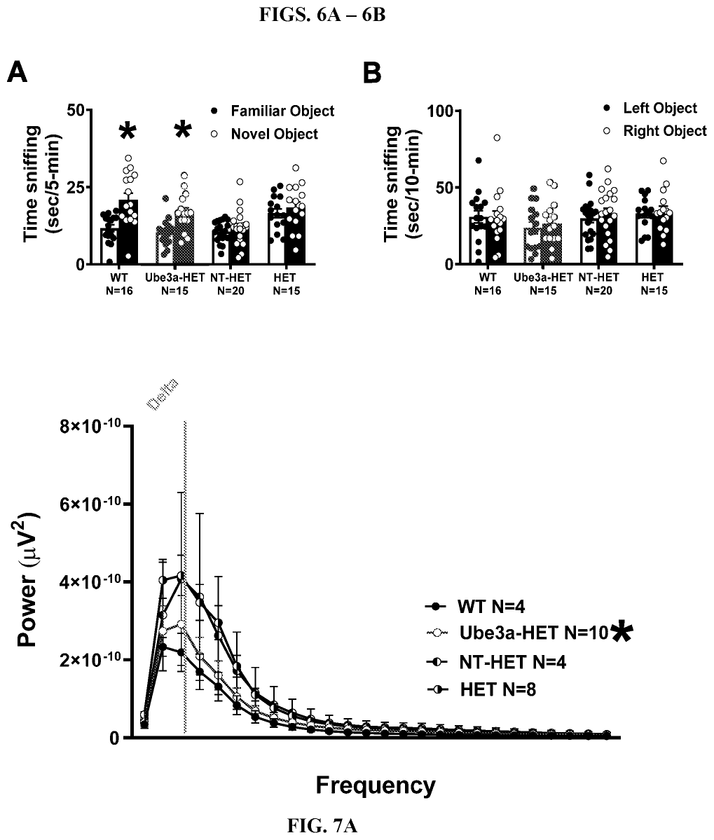

Ube3a-deficient mice display a lack of recognizing novel objects due to their neurological defects. Therefore, to evaluate whether the transplantation of Ube3a vector transduced human CD34+ HSC improved neurological deficits, the assay NOR was performed. This version of NOR started with animal habituation to the testing arena for 30-min. After 24-hr, the subjects were given a 10-min familiarization session where time spent sniffing each object was recorded. The objects were then cleaned and after a 1-hr interval, the mice were placed back in the arena with a familiar object and a novel object. As displayed in FIGS. 6A-6B, Ube3a-deficient mice transplanted with the Ube3a vector transduced human CD34+ HSC (Ube3a Het) displayed significantly (p<0.05) better performance compared to Ube3a-deficient mice transplanted with nontransduced (NT Het) cells. Ube3a Het mice performed similar to WT mice. More details are discussed below.

The novel object recognition test was conducted as previously described (1-8) in opaque matte white (P95 White, Tap Plastics, Sacramento, Calif., USA) arenas (41 cm 1×41 cm w×30 cm h). The assay consisted of four sessions: a 30-min habituation session, a second 10-min habituation phase, a 10-min familiarization session, and a 5-min recognition test. On day 1, each subject was habituated to a clean empty arena for 30-min. 24-h later, each subject was returned to the empty arena for an additional 10-min habituation session. The mouse was then removed from testing arena and was placed in a clean temporary holding cage while two identical objects were placed in the arena. Subjects were returned to the testing arena and given a 10-min of familiarization period in which they had time to investigate the two identical objects. After the familiarization phase subjects were returned to their holding cages for a 1-h interval period. One familiar object and one novel object were placed in the arena, where the two identical objects had been located during the familiarization phase. After the 1-h interval, each subject was returned to the arena for a 5-min recognition test. The familiarization session and the recognition test were recorded using Ethovision XT video tracking software (version 9.0, Noldus Information Technologies, Leesburg, Va., USA). Sniffing was defined as head facing the object with the nose point within 2 cm or less from the object. Time spent sniffing each object was scored by an investigator blind to both genotype and treatment. Recognition memory was defined as spending significantly more time sniffing the novel object compared to the familiar object. Total time spent sniffing both objects was used as a measure of general exploration. Time spent sniffing two identical objects during the familiarization phase confirmed the lack of an innate side bias. Within genotype repeated-measures ANOVA was used to analyze novel object recognition using novel versus familiar objects as comparison. F, degrees of freedom, and p-values are reported.

The AS Ube3a-deficient mice exhibit learning and memory deficits in the novel object recognition assay. (1-8) As displayed in FIGS. 6A-6B, functional reversal of cognitive behavioral impairments were observed in the BGU mice transplanted with human CD34+ HSC transduced with the Ube3a lentiviral vector. All scoring was initially performed by automated Ethovision software and the confirmed by manual scoring with a highly trained observer blinded to genotype and treatment group. As anticipated, the WT spent more time investigating the novel object versus the familiar object. In contrast, the HET and NT-HET groups did not exhibit typical novel object preference (FIG. 6A: WT; t (15)=8.714, p<0.0001; HET; t (14)=44.10, p<0.0002; NT-HET; t (19)=4.544, p<0.0060). In contrast to the HET and NT-HET groups, Applicant observed cognitive rescue in the Ube3a-HET treated group (FIG. 6A: Ube3a-HET; t (16)=3.271, p<0.003 All groups explored the two identical objects similarly during the familiarization phase (FIG. 6B: WT; HET; NT-HET; Ube3a-HET).

Electroencephalogram (EEG) Analysis

Several EEG abnormalities have been described in AS including elevated delta power. These observations are also present in the Ube3a−/+ BGU mouse model. Therefore, to evaluate whether the transplantation of Ube3a vector transduced cells improved EEG phenotypes and decreased the delta wave spectrum, an EEG analysis was performed.

EEG Implantation: Wireless EEG transmitters were implanted in anesthetized test animals using continuous isoflurane. Implants were placed in a subcutaneous pocket lateral to the spine to avoid discomfort of the animal and displacement due to movement. Each implant has two channels that include a signal and reference lead made of a Nickel-Colbalt based alloy insulated in medical-grade silicone. EEG, EMG, temperature, activity, and signal strength data were collected with each implant. To collect EEG data, two 1.0 mm burr holes were drilled (1.0 mm anterior and 1.0 mm lateral; −3.0 mm posterior and 1.0 mm lateral) relative to bregma and biopotential leads were secured using stainless steel skull screws. Once in place, the skull screws and lead connections were secured using dental cement. To collect EMG data, leads were placed in the trapezius muscles of the animal. Mice were given Carpofen (5 mg/kg; i.p.) directly after surgery and 24 hours post-surgery as an analgesic. Subjects were individually caged with ad libitum access to food and water for 1 week before EEG acquisition and monitored daily to ensure proper incision healing and recovery.

EEG Data Acquisition, Processing, and Analysis: After a 1-week recovery from surgical implantation, individually housed mice were assigned to PhysioTel RPC receiver plates that transmitted data from the EEG implants to a computer via the data exchange matrix using Ponemah software (Data Sciences International). EEG and EMG data were collected at a sampling rate of 500 Hz with a 0.1 Hz high-pass and 100 Hz low-pass bandpass filter. Activity, temperature and signal strength were collected at a sampling rate of 200 Hz. Data acquired in Ponemah was read into Python and further processed with a bandpass filter from 0-50 Hz to focus on frequencies of interest. For spectral analysis, frequency bands were defined as delta 0.5-4 Hz, theta 5-9 Hz, alpha 9-12 Hz, beta 13-30 Hz, and gamma 30-50 Hz. Spectral power was analyzed using Welch's Method which windows over the signal and averages across spectral samples. Relative delta frequencies were calculated by dividing the mean delta density by total density per animal and averaging across genotype. Two-way repeated measures ANOVAs were used to analyze power spectral densities between genotypes and Sidak's multiple comparisons tests were used to test significance at each frequency point. F, degrees of freedom, and p-values are reported.

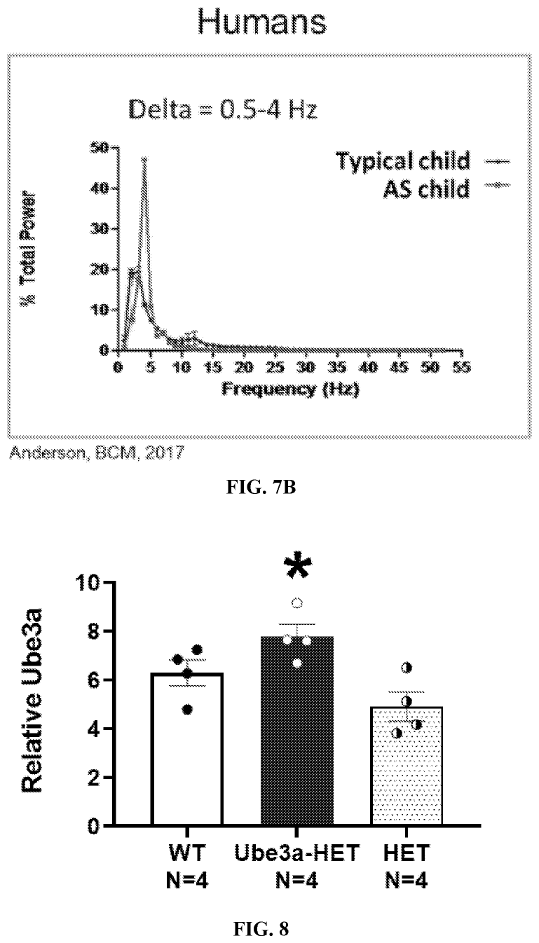

As displayed in FIG. 7A, Applicant observed a reduction of delta power in the Ube3a-HET cohort similar to the values of WT mice (F(25, 300)=0.223, p>0.999). The Ube3a-HET mice also displayed significantly lower delta power than HET (nontransplanted Ube3a−/+) mice (F(25, 475)=3.249, p<0.0001). Applicant observed elevated delta power in the HET group when compared to WT littermate controls which is consistent in mice with a Ube3a−/+ genotype (F(25, 362)=4.312, p<0.0001). While Applicant did not detect a significant change in the delta power of NT-HET animals (F(25, 150)=0.651, p=0.896), this was likely due to the high error rate detected in that group as the average line correlates to that of the Ube3a−/+ HET mice.

Expression of Ube3a in the CNS of Transplanted Ube3a−/+ Mice

To evaluate whether Ube3a could be detected in the CNS of Ube3a−/+ BGU mice transplanted with Ube3a vector transduced cells, brain sections obtained from mice were stained using a 3,3′-Diaminobenzidine (DAB) method.

Immunohistochemistry Labeling and Analysis: After evaluating the mice for functional improvements in AS phenotypes, the mice were euthanized and sagittal brain sections (40 um) were obtained from the bilateral mid-line. Tissues were labeled with ImmPACT DAB Peroxidase Substrate from Vector Labs, utilizing the Vectastain ABC Kit and in conformity with recommendations from the manufacturers included protocols. Tissues underwent peroxidase quenching using a 0.3% hydrogen peroxide solution in water for 30 minutes, followed by immersion in a 10% blocking solution in PBS, (SEA BLOCK Blocking Buffer) for one hour, which was followed by immersion in primary antibody UBE3a (Monoclonal Anti-UBE3A antibody produced in mouse, SAB1404508) at a concentration ratio of 1:500 with incubation overnight at 4° C. On the second day the tissues were immersed in a biotinylated secondary antibody solution at a concentration of 1:200 with incubation for 1 hour (Goat Anti-Mouse IgG Antibody, Vector Labs), followed by Vectastain ABC reagent incubation for 30 minutes (Vector Labs), and concluded with immersion into ImmPACT DAB Peroxidase Substrate (Vector Labs) at manufacturers recommended concentration for 8 minutes. In between each step all tissues were washed using PBST (0.1% Triton) for approximately 15 minutes. Serial sections were mounted onto uncharged slides and cover slipped using Permount Mounting Meduim. Bright field immunohistochemistry stained slides were scanned using the 20× objective (0.8, M27) with bright field illumination on an Axio Scan (Zeiss).

As displayed in FIG. 8, a significant (p=0.0126) increase in Ube3a expression was observed in the brains of Ube3a−/+ BGU mice transplanted with the Ube3a vector transduced cells as compared to nontransplanted Ube3a−/+ mice (HET). The levels of Ube3a expression in the brains of mice transplanted with the Ube3a vector transduced cells were similar to expression levels in WT mice and were not significantly different (p=0.1923). Statistical analyses were performed using Tukey's multiple comparisons test.

Taken together, the above data strongly demonstrates that after transplantation of newborn Ube3a−+/BGU mice with human CD34+ HSC transduced with a Ube3a expressing lentiviral vector, improvement in motor, behavior, and cognitive function was observed and was similar to WT mice. A normal EEG delta wave was also observed in the Ube3a−/+ mice transplanted with the Ube3a vector transduced cells. Expression of Ube3a, similar to levels observed in WT mice, was detected in Ube3a−/+ mice transplanted with the Ube3a vector transduced cells. These results demonstrate that by transplanting the therapeutic cells early on in the life of the mice prior to the development of clinical AS symptoms, the AS phenotypes can be prevented due to the recovery of Ube3a expression. These results also demonstrate the successful engraftment and functionality of the human CD34+ HSC transduced with the Ube3a expressing lentiviral vector as they were able to be detected in the peripheral blood and demonstrate expression of Ube3a in the brains of transplanted mice.

### Experiment No. 3—In Vivo Safety and Multi-Lineage Hematopoiesis and In Vitro Immortalization Assay of Ube3a Vector Transduced Human CD34+ HSC

Upon transduction of human CD34+ HSC with the Ube3a expressing lentiviral vector and the subsequent overexpression of Ube3a, it is possible that the in vivo engraftment and multi-lineage hematopoiesis potential of these cells could be compromised. Therefore, an in vivo model system able to mimic human CD34+ HSC engraftment and multi-lineage hematopoiesis should be used. The NOD-RAG1−/−IL2rg−/− (NRG) immunodeficient mouse model is ideal to evaluate these attributes of human CD34+ HSC. Due to the deletions of the RAG1 and IL2 gamma receptor genes, this model allows for the engraftment of human CD34+ HSC and the long term development of mature human cells including T cells, B cells, and macrophages in the peripheral blood and lymphoid organs including the spleen, thymus, and bone marrow. Due to these characteristics of the NRG mice, this model was used to evaluate the safety of the lentiviral vector transduction and the overexpression of Ube3a in human CD34+ HSC.

NOD-RAG1−/−IL2rg−/− (NRG) mice (stock number 007799) were obtained from The Jackson Laboratory. (9) Two to five-day old NRG mice were sublethally irradiated with 100 rads and transplanted with nontransduced (N=8), EGFP control vector (FIG. 1B) transduced (N=8), or hAS8 Ube3a lentiviral vector transduced (N=8) (FIG. 1D) (MOI 20 with 8 ug/ml protamine sulfate) human CD34+ HSC (300,000 total cells). As described above, the hAS8 vector expresses human Ube3a isoform 1 which has been modified to contain a secretion signal at the N-terminus and eight N-glycosylation sites throughout the protein. Human Ube3a isoform 1 is the species-equivalent to mouse Ube3a isoform 3 which had been used in preclinical studies in the BGU mouse model. The vectors containing the EGFP reporter genes were used to distinguish the Ube3a vector transduced cells from the nontransduced cells and to specifically gate on these cells when the flow cytometry analyses were performed. This gating strategy allowed Applicant to single out the vector transduced cells and specifically look at their development and differentiation into the various immune cells analyzed. To determine engraftment levels, at 3 months post-transplant, mice were bled via the tail vein and analyzed by flow cytometry with a PE-CY7-conjugated anti-human CD45 antibody. Flow cytometry was performed using a Beckman Coulter FC-500. Mice were used in accordance with institutional and IACUC guidelines. Once successful engraftment was observed, the mice were left for another 3 months (6 months total post-transplant) prior to euthanization to evaluate the multi-lineage hematopoiesis and lymphoid organ engraftment. Human T cell analysis (CD3, CD4, and CD8 cell markers) was performed on the blood, spleen, and thymus. Human B cell analysis (CD19) was performed on the spleen and bone marrow. Human macrophage (CD14) analysis was performed on the bone marrow. Human CD34+ analysis (CD34) was performed on the bone marrow. Cells from the spleen, thymus, peripheral blood, and bone marrow were labeled with antibodies specific for human immune cell markers and analyzed by flow cytometry. Flow cytometry was performed using a Beckman Coulter FC500. Cells were initially gated on EGFP to identify the vector transduced cells and then analyzed for the human cell specific markers to identify the development of the specific cell types.

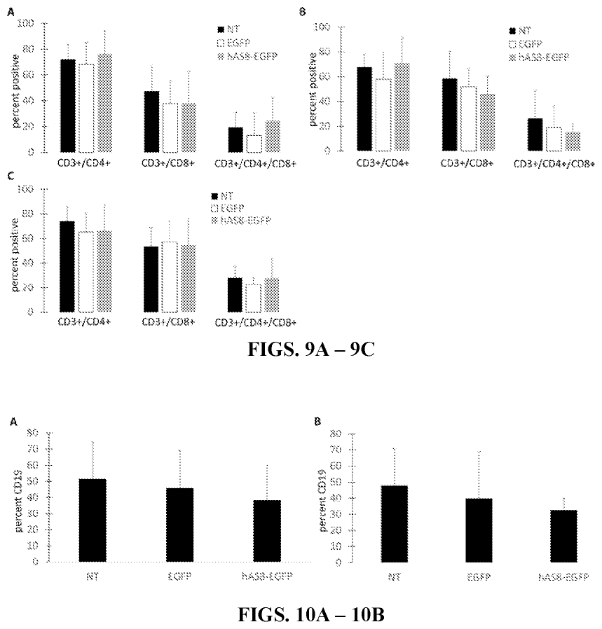

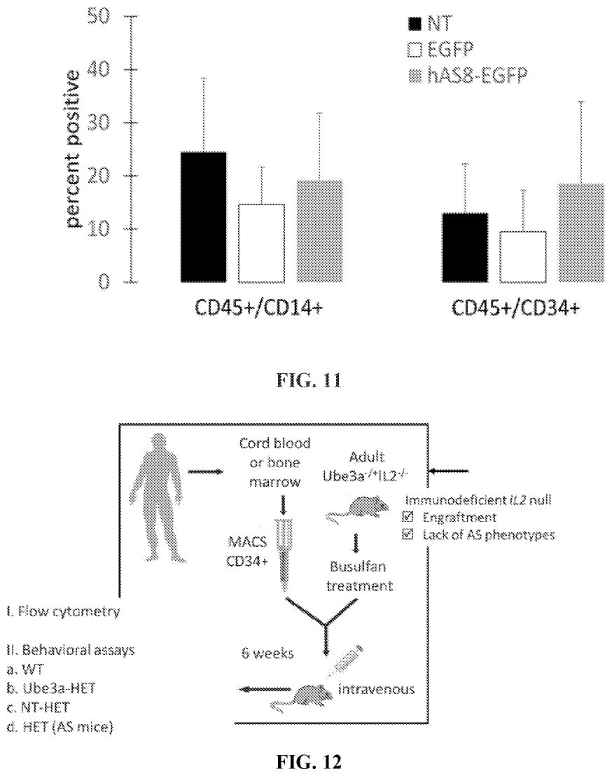

Analyses determined that normal engraftment of hAS8 Ube3a lentiviral vector transduced CD34+ cells and development of human T cells was demonstrated in the peripheral blood of engrafted NRG mice. As displayed in FIG. 9A, no significant difference (p>0.05) in the development of CD3+/CD4+ T cells, CD3+/CD8+ T cells, or CD4+/CD8+ double positive T cells from hAS8 vector transduced human CD34+ cells was observed compared to EGFP-control vector transduced or nontransduced human CD34+ cells. Similar levels of all T cells populations analyzed were observed in the peripheral blood of all mouse cohorts. On average, mice transplanted with the Ube3a vector transduced cells displayed CD3+/CD4+(76.1%), CD3+/CD8+(38.2%), and CD3+/CD4+/CD8+(24.4%) levels in the peripheral blood compared to mice transplanted with nontransduced cells (CD3+/CD4+(72.1%), CD3+/CD8+(47.5%), and CD3+/CD4+/CD8+(19.6%)) and EGFP alone vector transduced cells (CD3+/CD4+(68.2%), CD3+/CD8+(37.9%), and CD3+/CD4+/CD8+(13.1%)). Similar to the peripheral blood, as displayed in FIG. 9B, no significant difference (p>0.05) in the development of CD3+/CD4+ T cells, CD3+/CD8+ T cells, or CD4+/CD8+ double positive T cells in the spleens of engrafted mice from hAS8 vector transduced human CD34+ cells was observed compared to EGFP-control vector transduced or nontransduced human CD34+ cells. On average, mice transplanted with the Ube3a vector transduced cells displayed CD3+/CD4+(70.9%), CD3+/CD8+(46.3%), and CD3+/CD4+/CD8+(15.3%) levels in the spleen compared to mice transplanted with nontransduced cells (CD3+/CD4+(67.8%), CD3+/CD8+(58.5%), and CD3+/CD4+/CD8+(26.4%)) and EGFP alone vector transduced cells (CD3+/CD4+(57.9%), CD3+/CD8+(52.0%), and CD3+/CD4+/CD8+(19.1%)). Applicant next analyzed the levels of T cells in the thymus of engrafted mice. As displayed in FIG. 9C, no significant difference (p>0.05) in the development of CD3+/CD4+ T cells, CD3+/CD8+ T cells, or CD4+/CD8+ double positive T cells in the thymus of engrafted mice from hAS8 vector transduced human CD34+ cells was observed compared to EGFP-control vector transduced or nontransduced human CD34+ cells. On average, mice transplanted with the Ube3a vector transduced cells displayed CD3+/CD4+(66.1%), CD3+/CD8+(54.4%), and CD3+/CD4+/CD8+(27.7%) levels in the thymus compared to mice transplanted with nontransduced cells (CD3+/CD4+(74.3%), CD3+/CD8+(53.6%), and CD3+/CD4+/CD8+(28.0%)) and EGFP alone vector transduced cells (CD3+/CD4+(65.2%), CD3+/CD8+(57.1%), and CD3+/CD4+/CD8+(22.3%)). These results demonstrate that human CD34+ HSC transduced with the hAS8 Ube3a expressing lentiviral vector were capable of engraftment in the NRG mice and were able to differentiate into normal T cells in the peripheral blood, spleen, and thymus of engrafted mice.

As a next step in evaluating the safety of the Ube3a vector transduced cells, human B cell analyses were performed on the spleen and bone marrow of engrafted NRG mice. As displayed in FIG. 10A, no significant difference (p>0.05) in the development of CD45+/CD19+ B cells in the spleen of engrafted mice from hAS8 vector transduced human CD34+ cells was observed compared to EGFP-control vector transduced or nontransduced human CD34+ cells. On average, mice transplanted with the Ube3a vector transduced cells displayed CD45+/CD19+(38.5%) levels in the spleen compared to mice transplanted with nontransduced cells (CD45+/CD19+(51.6%) and EGFP alone vector transduced cells (CD45+/CD19+(45.8%). Similarly, as displayed in FIG. 10B, no significant difference (p>0.05) in the development of CD45+/CD19+ B cells in the bone marrow of engrafted mice from hAS8 vector transduced human CD34+ cells was observed compared to EGFP-control vector transduced or nontransduced human CD34+ cells. On average, mice transplanted with the Ube3a vector transduced cells displayed CD45+/CD19+(32.6%) levels in the bone marrow compared to mice transplanted with nontransduced cells (CD45+/CD19+(47.9%) and EGFP alone vector transduced cells (CD45+/CD19+(39.8%). These results demonstrate that human CD34+ HSC transduced with the hAS8 Ube3a expressing lentiviral vector were capable of engraftment in the NRG mice and were able to differentiate into normal B cells in the spleen and bone marrow of engrafted mice.

Applicant next evaluated the levels of human macrophages and human CD34+ cells engrafted in the bone marrow of NRG mice transplanted with hAS8 Ube3a vector transduced cells. As displayed in FIG. 11, no significant difference (p>0.05) in the development of CD45+/CD14+ macrophages or CD45+/CD34+ cells in the bone marrow of engrafted mice from hAS8 vector transduced human CD34+ cells was observed compared to EGFP-control vector transduced or nontransduced human CD34+ cells. On average, mice transplanted with the Ube3a vector transduced cells displayed CD45+/CD14+(19.2%) levels and CD45+/CD34+(18.5%) levels in the bone marrow compared to mice transplanted with nontransduced cells (CD45+/CD14+(24.5%) and CD45+/CD34+(13.0)) and EGFP alone vector transduced cells (CD45+/CD14+(14.6%) and CD45+/CD34+(9.5)). These results demonstrate that human CD34+ HSC transduced with the hAS8 Ube3a expressing lentiviral vector were capable of engraftment in the NRG mice and were able to differentiate into normal macrophages in the bone marrow of engrafted mice. These results also demonstrate that human CD34+ cells transduced with the Ube3a lentiviral vector were still present in the bone marrow of engrafted mice six months post-transplant.

In Vitro Immortalization Assay

To evaluate if the transduction of human CD34+ cells with the hAS8 Ube3a expressing lentiviral vector caused any immortalization of the cells, an in vitro immortalization assay was performed. Briefly, human CD34+ cells were left nontransduced or transduced with either the EGFP alone control vector, the hAS8-GFP vector, or the hAS8 lentiviral vector (FIG. 1). These cells were cultured for 14 days in IMDM media containing 10% FBS and supplemented with 50 ng/ml SCF, Flt-3 ligand, and TPO. Cell density was adjusted to 5×10⁵ cells/ml every 3 days. After this expansion, cells were plated in 3×96-well plates per cell group at a density of 100 cells/ml and left for 14 days. Wells were subsequently counted for the frequency of immortalization. A total of 288 wells per cell group were plated and counted for the frequency of immortalization.

TABLE 1
In vitro immortalization assay with Ube3a
lentiviral vector transduced cells

| Cell Type | Positive clones |
|---|---|
| Nontransduced | 0/288 |
| EGFP-alone lentivector transduced | 0/288 |
| hAS8-EGFP lentivector transduced | 0/288 |
| hAS8 lentivector transduced | 0/288 |

### Experiment No. 4—Functional Efficacy of HSC Following Adult Treatment with HSC Transduced with the Ube3a Lentiviral Vector

As a next step in evaluating the efficacy of the Ube3a lentiviral vector transduced human CD34+ HSC, adult BGU mice were transplanted with the cells after AS phenotypes had developed. To evaluate the adult mice, the cells were transplanted intravenously at 4-5 weeks of age which is the age of the BGU after AS phenotypes had already developed.

Functional Rescue of AS Phenotypes in Adult BGU Mice Transplanted with Ube3a Vector Transduced Cells

To evaluate the ability of the Ube3a lentiviral vectors to improve AS phenotypes after their appearance, mAS8 vector transduced human CD34+ HSC were transplanted into adult immunodeficient BGU mice. For the adult mice, at 4-5 weeks of age, the mice were treated with 20 mg/kg busulfan intraperitonially 48 and 24 hours prior to transplanting them with 500,000 total cells/mouse intravenously, either nontransduced (NT) or Ube3a (Ube3a-HET) lentiviral vector transduced human CD34+ HSC. Six weeks post-transplant, mice were bled via the tail vein and analyzed for engraftment by flow cytometry using a mouse anti-human CD45 antibody. Successfully engrafted mice were then evaluated for behavioral phenotypes. The order and age of testing were as follows: (1) open field at 11-12 weeks of age, (2) beam walking at 11-12 weeks of age, (3) rotarod at 12-13 weeks of age, (4) digigait at 12-13 weeks of age, and (5) novel object recognition at 14-15 weeks of age.

The methods for the open field, beam walking, rotarod, DigiGait, and novel object recognition assays were performed as described above.

Testing Cohorts

The same four testing cohorts were used for the adult efficacy experiments: WT (wild-type expression of Ube3a), HET (BGU mice deficient for Ube3a), NT-HET (BGU mice deficient for Ube3a transplanted with nontransduced human CD34+ HSC), and Ube3a-HET (BGU mice deficient for Ube3a transplanted with Ube3a lentiviral vector transduced human CD34+ HSC). Sexes were combined since there has never been a sex difference in Angelman Syndrome preclinical or clinical outcomes.

Adult HET mice that were treated with either nontransduced HSC (NT-Het) or Ube3a vector transduced (Ube3a-HET) human CD34+ HSC were behaviorally tested six-weeks post-transplant. Similar to the neonatal study as disclosed herein, Applicant performed multiple assays of a tailored motoric behavioral battery.

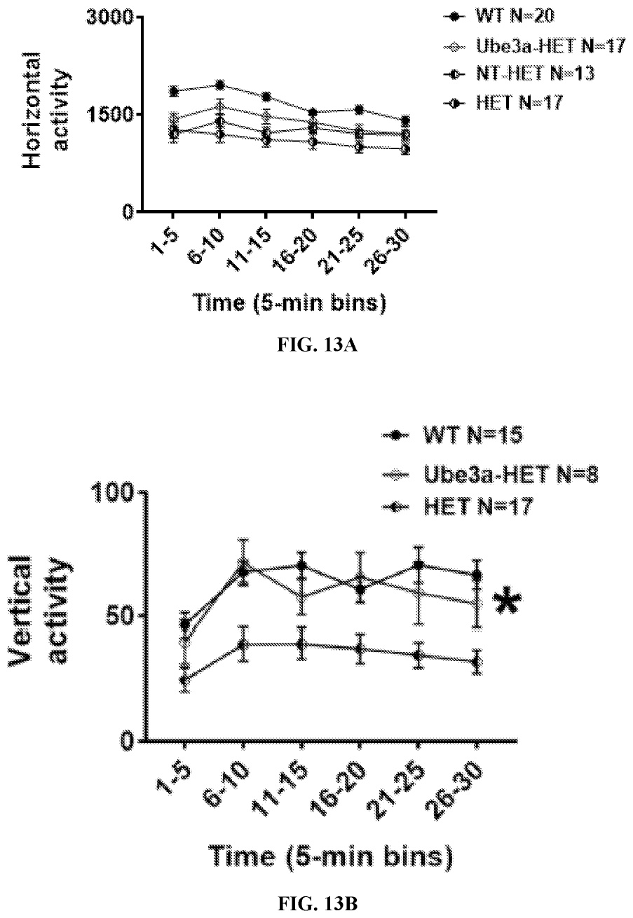

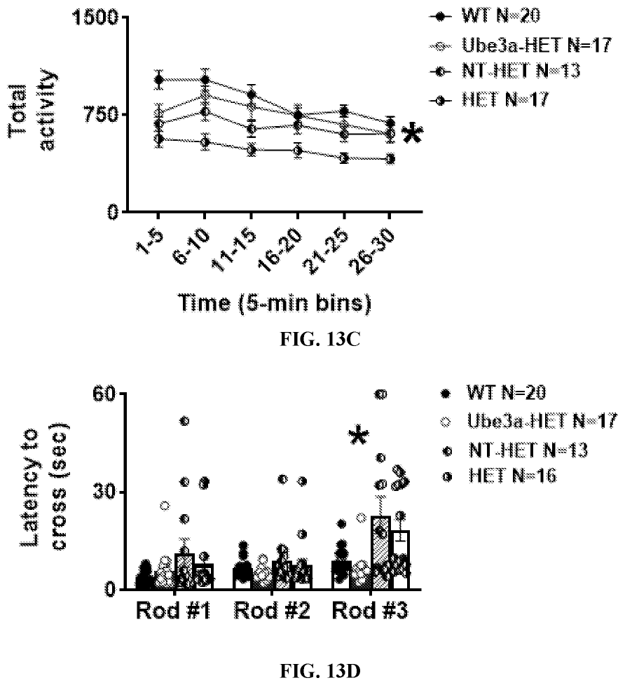

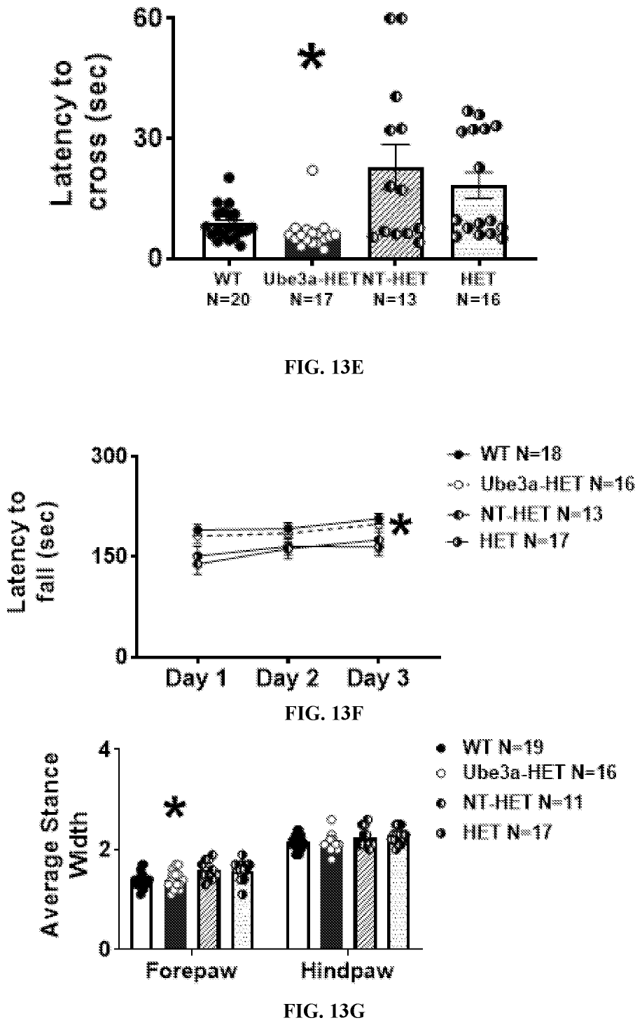

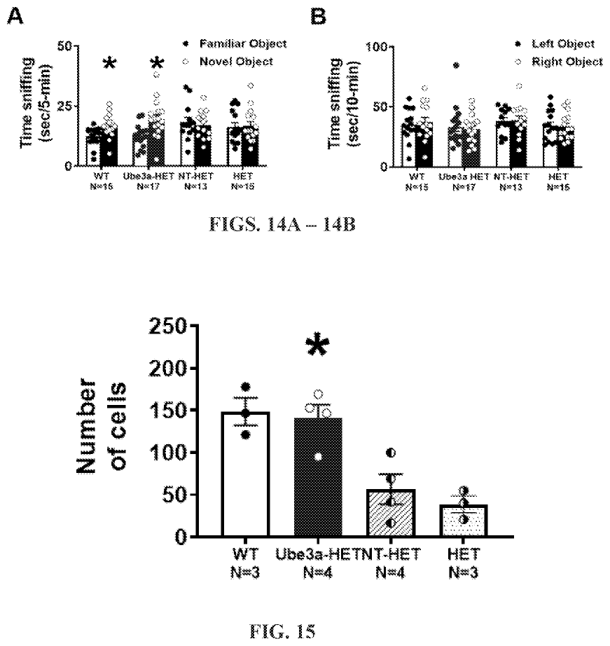

Locomotion in a novel open field was collected assessing the total distance traversed and the horizontal movements using beam breaks in a novel arena. Group differences in total distance were observed using a multi-factor repeated measures ANOVA (FIG. 13C; F (3, 63)=9.650, p<0.0001), followed by the Holm Sidak posthoc test (p=0.3465). As in the treated pups, in the adult treated groups, Holm-Sidak corrected for multiple comparisons and still clearly highlighted that both NT-HET (p<0.0397) and HET (p<0.0001) differed from WT while the treatment group Ube3a-HET did not differ from WT (p=0.3465). Holm-Sidak corrected posthoc analysis for multiple comparisons highlighted that both NT-HET (p<0.0001) and HET (p<0.0002) differed from WT while the treatment group Ube3a-HET did not differ from WT (p=0.191). The Ube3a-treated HET do not differ from WT at any timepoint across the 30 minute assay (0-5 min, p=0.0122, 6-10 min, p=0.4324, 11-15 min, p=0.6395, 16-20 min, p=>0.9999, 21-25 min p=0.5583, 26-30 min p=0.7356), while the HET, adjusted p's from Holm Sidak provided (0-5 min, p<0.0001, 6-10 min, p<0.0001, 11-15 min, p<0.0001, 16-20 min, p<0.0067, 21-25 min, p<0.0002, 26-30 min, p<0.0059) and differed in 4 of the 6 5-min time bins. NT-HETs differed in 3 of the 6 5-min time bins (0-5 min, p<0.0013, 6-10 min, p<0.0316, 11-15 min, p<0.017, 16-20 min, p=0.7957, 21-25 min, p=0.1765, 26-30 min, p=0.7682).

In corroboration, the horizontal activity counts also illustrated group differences using a multi-factor repeated measures ANOVA (FIG. 13A; F (3, 61)=11.78, p<0.0001). Holm-Sidak corrected posthoc analysis for multiple comparisons highlighted that both NT-HET (p<0.0001) and HET (p<0.0002) differed from WT while the treatment group Ube3a-HET did not differ from WT (p=0.191). The Ube3a-treated HET do not differ from WT at any timepoint across the 30 minute assay (0-5 min, p=0.4383, 6-10 min, p=0.3297, 11-15 min, p=0.0439, 16-20 min, p=0.2038, 21-25 min p=0.7833, 26-30 min p=2903), while the HET, adjusted p's from Holm Sidak provided (0-5 min, p<0.0001, 6-10 min, p<0.0001, 11-15 min, p<0.0023, 16-20 min, p<0.0002, 21-25 min, p<0.0231, 26-30 min, p<0.0214) differed in 5 of the 6 5-min time bins and the NT-HET differed on every 5 minute bin of the 30 minute task (0-5 min, p<0.0002, 6-10 min, p<0.0001, 11-15 min, p<0.0001, 16-20 min, p<0.0003, 21-25 min, p<0.0103, 26-30 min, p<0.0001).

A balance beam walking motor task was conducted as in the pup cohort, all groups showed longer latencies to cross the rod shaped beams as they became thinner and more difficult to traverse (F (2, 124)=11.73, p<0.0001), as expected. Group differences using a multi-factor repeated measures ANOVA (FIGS. 13D-13E; F (3, 62)=11.24, p<0.0001). Amazingly, Ube3a-deficient mice transplanted with the Ube3a lentivector transduced human CD34+ HSC (Ube3a-HET) exhibited wildtype values of performance on the beam, illustrated via Holm-Sidak posthoc analysis. The Ube3a-treated HET do not differ from WT on rod #3 (rod #3, p=0.9931) while the HET (p<0.0164) and NT-HET (p<0.0002) had much slower latencies to cross rod #3, highlighting the improved motor coordination of the treated group. FIG. 13E illustrates the fastest groups to cross the rods, highlighting WT and Ube3a-HET, as being indistinguishable from one another by time across rod, highlighting substantial improvement in motor coordination.

On the rotarod, the corroborating coordination assay, HET mice differed from WT in their latencies to fall from the accelerating rod (FIG. 13F; F (3, 59)=3.30, p<0.05) on day 3 (p<0.05), illustrating poor motor coordination and a lack of motor learning in the Ube3a-1+ HET mice. Strikingly, Ube3a−/+ mice transplanted with the Ube3a lentivector transduced human CD34+ HSC were similar to wildtype across all three days of testing using Holm-Sidak posthoc analyses (Day 1; p=0.9321), (Day 2; p=0.9691), and (Day 3; p=0.9514). Group differences were detected using a multi-factor ANOVA (FIG. 20F; F (3, 65)=5.782, p<0.002). As displayed in FIG. 13G, DigiGait analysis demonstrated that HET (p<0.0026) and NT-HET (p<0.002) differ from wildtype while the Ube3a-treated HET group showed a narrowing of these wide stances (p=0.3486).

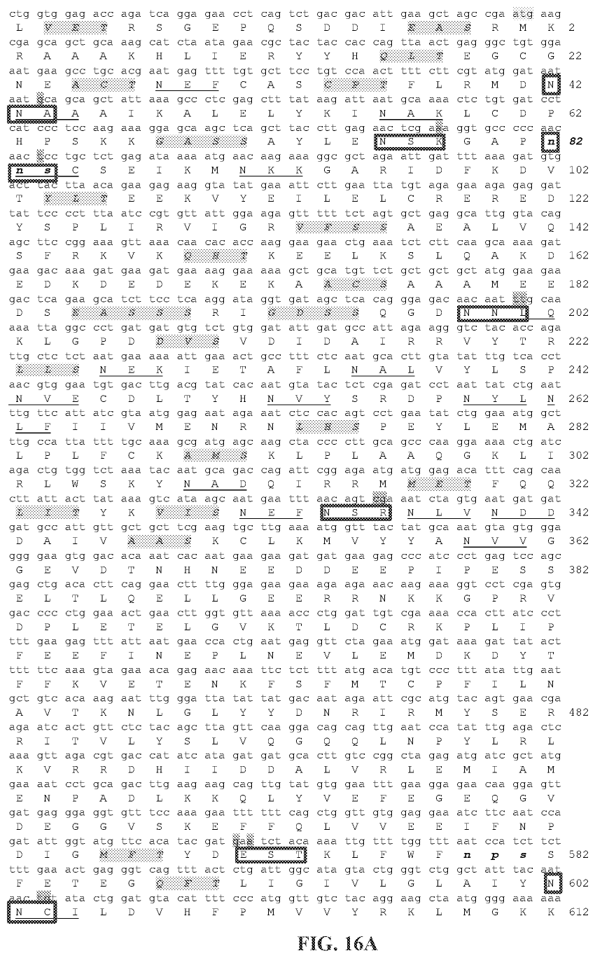

The data illustrated functional reversal of cognitive behavioral impairments in FIGS. 14A-14B following six weeks post-transplant with human CD34+ HSC transduced with a lentiviral vector expressing Ube3a. Cognitive abilities as measured by the NOR task were tested in several independent cohorts to reach the desired sample size, however, each sub cohort contained each treatment group, to maximize standards the experimental design. All scoring was initially performed by automated Ethovision software and the confirmed by manual scoring with a highly trained observer blinded to genotype and treatment group. As anticipated, the WT spent more time investigating the novel object versus the familiar object. In contrast, the HET and NT-HET groups did not exhibit typical novel object preference (FIG. 14A: WT; t (14)=2.626, p<0.0139; HET; t (16)=0.1392, p>0.8464; NT-HET; t (12)=0.4597, p>0.6553). Impressively, Applicant observed cognitive rescue in the Ube3a-HET treated group (FIG. 23A: Ube3a-HET; t (14)=3.271, p<0.0026. All groups explored the two identical objects similarly during the familiarization phase (FIG. 14B: WT; t (14)=0.3698, p=0.7193; HET; t (16)=0.2968, p=0.8903; NT-HET; t (12)=0.1331, p=0.8952; Ube3a-HET; t (14)=0.1392, p=0.6488).

Expression of Ube3a in Adult Ube3a−/+ Mice Transplanted with Ube3a Vector Transduced Cells

To evaluate whether Ube3a was expressed in the brains of adult Ube3a−/+ mice transplanted with Ube3a vector transduced cells, a DAB/anti-Ube3a antibody detection assay was performed. The methods for this assay are described above. As displayed in FIG. 15, a significant increase (p=0.0058 and p=0.0124) in Ube3a expression was observed in the brains of Ube3a−/+ mice transplanted with Ube3a vector transduced cells (Ube3a-HET) as compared to nontransplanted Ube3a−/+ mice (HET) and Ube3a−/+ mice transplanted with nontransduced cells (NT-HET), respectively. The levels of Ube3a expression in the Ube3a-HET cohort was similar to the expression levels in WT BGU mice (p=0.9867). Statistical analyses were performed using Tukey's multiple comparisons test.

Taken together, the above data strongly demonstrates that after intravenous transplantation of adult Ube3a−/+ BGU mice with human CD34+ HSC transduced with a Ube3a expressing lentiviral vector, correction of already developed AS phenotypes was achieved. Mice receiving the Ube3a expressing cells improved in their motor, behavior, and cognitive functions to levels similar to WT mice. Expression levels of Ube3a in the brains of Ube3a−/+ mice were also restored to WT levels after transplantation of the Ube3a vector transduced cells. These results demonstrate that by transplanting the therapeutic cells after AS phenotypes have developed, these phenotypes can be corrected. These results also demonstrate the successful engraftment and functionality of the human CD34+ HSC transduced with the Ube3a expressing lentiviral vector as they were able to be detected in the peripheral blood and demonstrate expression of Ube3a in the brains of mice transplanted via the intravenous route.

### Experiment No. 5—Clinical

Applicant also demonstrated that the transduction of the human CD34+ HSC and the subsequent overexpression of Ube3a did not have a detrimental effect on CD34+ cell function, engraftment, or differentiation into normal immune cells as displayed in the CFU assay, the further differentiation of in vitro derived phenotypically normal macrophages, and the in vivo engraftment and further differentiation of T cells, B cells, and macrophages in NRG mice.

HSC Mobilization and Peripheral Stem Cell Collection

Insertion of a central venous catheter (CVC) is required for all apheresis procedures. Patients undergo stem cell mobilization using 5 consecutive days of subcutaneous injections of 10 μg/kg of G-CSF. Plerixafor 240 μg/kg/dose is allowed to improve the collection outcome based on the discretion of the treating physicians and is administered 4-6 hours before the apheresis starts. Routine monitoring during the collection, patient supportive care, vital signs, and CBC monitoring follow institutional guidelines. On the day of collection, the subject receives a subcutaneous injection of plerixafor (240 μg/kg) 5 hours before collection.

Subjects can undergo up to 2 mobilization cycles to achieve an adequate cell dose. Insertion of a temporary apheresis catheter is strongly recommended to facilitate the HSPC harvest.

The Drug Product cell dose is selected from the following for each subject and satisfy all the release criteria: at least about 1×10⁴ CD34+ cells/kg, or at least about 2×10⁴ CD34+ cells/kg, or at least about 3×10⁴ CD34+ cells/kg, or at least about 4×10⁴ CD34+ cells/kg, or at least about 5×10⁴ CD34+ cells/kg, or at least about 6×10⁴ CD34+ cells/kg, or at least about 7×10⁴ CD34+ cells/kg, or at least about 8×10⁴ CD34+ cells/kg, or at least about 9×10⁴ CD34+ cells/kg, or at least about 1×10⁵ CD34+ cells/kg, or at least about 2×10⁵ CD34+ cells/kg, or at least about 3×10⁵ CD34+ cells/kg, or at least about 4×10⁵ CD34+ cells/kg, or at least about 5×10⁵ CD34+ cells/kg, or at least about 6×10⁵ CD34+ cells/kg, or at least about 7×10⁵ CD34+ cells/kg, or at least about 8×10⁵ CD34+ cells/kg, or at least about 9×10⁵ CD34+ cells/kg, or at least about 1×10⁶ CD34+ cells/kg, or at least about 2×10⁶ CD34+ cells/kg, or at least about 3×10⁶ CD34+ cells/kg, or at least about 4×10⁶ CD34+ cells/kg, or at least about 5×10⁶ CD34+ cells/kg, or at least about 6×10⁶ CD34+ cells/kg, or at least about 7×10⁶ CD34+ cells/kg, or at least about 8×10⁶ CD34+ cells/kg, or at least about 9×10⁶ CD34+ cells/kg, or at least about 1×10⁷ CD34+ cells/kg, or at least about 2×10⁷ CD34+ cells/kg, or at least about 3×10⁷ CD34+ cells/kg, or at least about 4×10⁷ CD34+ cells/kg, or at least about 5×10⁷ CD34+ cells/kg, or at least about 6×10⁷ CD34+ cells/kg, or at least about 7×10⁷ CD34+ cells/kg, or at least about 8×10⁷ CD34+ cells/kg, or at least about 9×10⁷ CD34+ cells/kg, or at least about 1×10⁸ CD34+ cells/kg, or at least about 2×10⁸ CD34+ cells/kg, or at least about 3×10⁸ CD34+ cells/kg, or at least about 4×10⁸ CD34+ cells/kg, or at least about 5×10⁸ CD34+ cells/kg, or at least about 6×10⁸ CD34+ cells/kg, or at least about 7×10⁸ CD34+ cells/kg, or at least about 8×10⁸ CD34+ cells/kg, or at least about 9×10⁸ CD34+ cells/kg, or at least about 1×10⁹ CD34+ cells/kg body weight.

HSC Dose

In some embodiments, the dose is less than 1×10⁹, or less than 9×10⁸, or less than 8×10⁸, or less than 7×10⁸, or less than 6×10⁸, or less than 5×10⁸, or less than 4×10⁸, or less than 3×10⁸, or less than 2×10⁸, or less than 1×10⁸, or less than 9×10⁷, or less than 8×10⁷, or less than 7×10⁷, or less than 6×10⁷, or less than 5×10⁷, or less than 4×10⁷, or less than 3×10⁷, or less than 2×10⁷, or less than 1×10⁷, or less than 9×10⁶, or less than 8×10⁶, or less than 7×10⁶, or less than 6×10⁶, or less than 5×10⁶, or less than 4×10⁶, or less than 3×10⁶, or less than 2×10⁶, or less than 1×10⁶, or less than 9×10⁵, or less than 8×10⁵, or less than 7×10⁵, or less than 6×10⁵, or less than 5×10⁵, or less than 4×10⁵, or less than 3×10⁵, or less than 2×10⁵, or less than 1×10⁵ CD34+ HSC/kg body weight.

In some embodiments, the dose is less than 1×10⁹, or less than 9×10⁸, or less than 8×10⁸, or less than 7×10⁸, or less than 6×10⁸, or less than 5×10⁸, or less than 4×10⁸, or less than 3×10⁸, or less than 2×10⁸, or less than 1×10⁸, or less than 9×10⁷, or less than 8×10⁷, or less than 7×10⁷, or less than 6×10⁷, or less than 5×10⁷, or less than 4×10⁷, or less than 3×10⁷, or less than 2×10⁷, or less than 1×10⁷, or less than 9×10⁶, or less than 8×10⁶, or less than 7×10⁶, or less than 6×10⁶, or less than 5×10⁶, or less than 4×10⁶, or less than 3×10⁶, or less than 2×10⁶, or less than 1×10⁶, or less than 9×10⁵, or less than 8×10⁵, or less than 7×10⁵, or less than 6×10⁵, or less than 5×10⁵, or less than 4×10⁵, or less than 3×10⁵, or less than 2×10⁵, or less than 1×10⁵ gene modified CD34+/kg body weight.

Cells can be administered between 72 to 84 hours after the final dose of IV busulfan or myoablative therapy

Treatment

The preparative regimen described in Table 2 is exemplary of a treatment protocol.

TABLE 2

| Days | Treatment |
|---|---|
| −7 | CVC insertion, commence anti-convulsant |
| | prophylaxis |
| −6 to −3 | Conditioning: Busulfan (BU) 3.2 mg/kg IV daily* |
| −2 | Rest |
| −1 | Rest |
| 0 | Infusion: autologous drug product |

*BU pharmacokinetics is performed after the 1^{st} and 3^{rd} dose per routine. For daily administration a 3-hour infusion commencing at 0600 permits shipment of plasma samples for BU PK studies at the reference site. Samples are obtained at the end of the 3-hour infusion, then at 15 min, 1 hour, 2 hours, and 4 hours after finishing the infusion.

Infection Prophylaxis

Patients can receive infection prophylaxis and nutritional support. Infection prophylaxis includes, but is not limited to, agents or strategies (e.g., PCR screening and preemptive therapy) to reduce the risk of bacterial, herpes simplex, CMV, HHV-6, EBV, Pneumocystis jiroveci, and fungal infections.

Indwelling Central Venous Catheter

A double lumen central venous catheter can be inserted at the time of apheresis and remains inserted during the transplant to give IV medications, transfuse blood products, and to administer the stem cells. This catheter may be removed and replaced as clinically indicated. However, the graft must be infused through a central line.

Preparative Regimen for Transplant

Patients can undergo myeloablative conditioning therapy with intravenous (IV) busulfan. Single agent busulfan at 3.2 mg/kg once a day for 4 days is administered intravenously through a central venous catheter as the myeloablative conditioning regimen. This regimen has been used with successful engraftment and acceptable toxicity in multiple gene therapy stem cell transplant trials.

An exemplified transplant timeline is provided below:
1. Single agent busulfan on days −5 to −3.
2. Gene modified hematopoietic stem cells are thawed if frozen and infused on Day 0, at least 72 hours after the last dose of busulfan.
3. Back up hematopoietic stem cells can be infused after day 30 in patients with ANC<500 and on or after day 20 in patients with ANC <500 and a life-threatening complications.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

It should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Y. H. Jiang, D. Armstrong, U. Albrecht, C. M. Atkins, J. L. Noebels, G. Eichele, J. D. Sweatt, A. L. Beaudet, Mutation of the Angelman ubiquitin ligase in mice causes increased cytoplasmic p53 and deficits of contextual learning and long-term potentiation. Neuron 21, 799-811 (1998).
2. M. C. Judson, M. L. Wallace, M. S. Sidorov, A. C. Burette, B. Gu, G. M. van Woerden, I. F. King, J. E. Han, M. J. Zylka, Y. Elgersma, R. J. Weinberg, B. D. Philpot, GABAergic Neuron-Specific Loss of Ube3a Causes Angelman Syndrome-Like EEG Abnormalities and Enhances Seizure Susceptibility. Neuron 90, 56-69 (2016).
3. H. S. Huang, A. J. Burns, R. J. Nonneman, L. K. Baker, N. V. Riddick, V. D. Nikolova, T. T. Riday, K. Yashiro, B. D. Philpot, S. S. Moy, Behavioral deficits in an Angelman syndrome model: effects of genetic background and age. Behavioural brain research 243, 79-90 (2013).
4. M. Sonzogni, I. Wallaard, S. S. Santos, J. Kingma, D. du Mee, G. M. van Woerden, Y. Elgersma, A behavioral test battery for mouse models of Angelman syndrome: a powerful tool for testing drugs and novel Ube3a mutants. Mol Autism 9, 47 (2018).
5. J. K. Gentile, W. H. Tan, L. T. Horowitz, C. A. Bacino, S. A. Skinner, R. Barbieri-Welge, A. Bauer-Carlin, A. L. Beaudet, T. J. Bichell, H. S. Lee, T. Sahoo, S. E. Waisbren, L. M. Bird, S. U. Peters, A neurodevelopmental survey of Angelman syndrome with genotype-phenotype correlations. J Dev Behav Pediatr 31, 592-601 (2010).
6. J. C. Grieco, A. Gouelle, E. J. Weeber, Identification of spatiotemporal gait parameters and pressure-related characteristics in children with Angelman syndrome: A pilot study. J Appl Res Intellect Disabil 31, 1219-1224 (2018).
7. W. H. Tan, C. A. Bacino, S. A. Skinner, I. Anselm, R. Barbieri-Welge, A. Bauer-Carlin, A. L. Beaudet, T. J. Bichell, J. K. Gentile, D. G. Glaze, L. T. Horowitz, S. V. Kothare, H. S. Lee, M. P. Nespeca, S. U. Peters, T. Sahoo, D. Sarco, S. E. Waisbren, L. M. Bird, Angelman syndrome: Mutations influence features in early childhood. Am J Med Genet A 155A, 81-90 (2011).
8. A. C. Wheeler, P. Sacco, R. Cabo, Unmet clinical needs and burden in Angelman syndrome: a review of the literature. Orphanet J Rare Dis 12, 164 (2017).
9. T Pearson, L D Shultz, D Miller, M King, J Laning, W Fodor, A Cuthbert, L Burzenski, B Gott, B Lyons, O Foreman, A A Rossini, D L Greiner, Non-obese diabetic-recombination activating gene-1 (NOD-Rag1 null) interleukin (IL)-2 receptor common gamma chain (IL2r gamma null) null mice: a radioresistant model for human lymphohaematopoietic engraftment. Clin Exp Immunol 154, 270-284 (2008).
10. K. Buiting, C. Williams, B. Horsthemke, Angelman syndrome—insights into a rare neurogenetic disorder. Nature reviews. Neurology 12, 584-593 (2016).
11. C. A. Williams, Neurological aspects of the Angelman syndrome. Brain Dev 27, 88-94 (2005).
12. C. A. Williams, The behavioral phenotype of the Angelman syndrome. Am J Med Genet C Semin Med Genet 154C, 432-437 (2010).
13. S. J. Chamberlain, M. Lalande, Neurodevelopmental disorders involving genomic imprinting at human chromosome 15q11-q13. Neurobiol Dis 39, 13-20 (2010).
14. T. Matsuura, J. S. Sutcliffe, P. Fang, R. J. Galjaard, Y. H. Jiang, C. S. Benton, J. M. Rommens, A. L. Beaudet, De novo truncating mutations in E6-AP ubiquitin-protein ligase gene (UBE3A) in Angelman syndrome. Nat Genet 15, 74-77 (1997).
15. T. Kishino, M. Lalande, J. Wagstaff, UBE3A/E6-AP mutations cause Angelman syndrome. Nat Genet 15, 70-73 (1997).
16. U. Albrecht, J. S. Sutcliffe, B. M. Cattanach, C. V. Beechey, D. Armstrong, G. Eichele, A. L. Beaudet, Imprinted expression of the murine Angelman syndrome gene, Ube3a, in hippocampal and Purkinje neurons. Nat Genet 17, 75-78 (1997).
17. R. F. Wynn, J. Mercer, J. Page, T. F. Carr, S. Jones, J. E. Wraith, Use of enzyme replacement therapy (Laronidase) before hematopoietic stem cell transplantation for mucopolysaccharidosis I: experience in 18 patients. J Pediatr 154, 135-139 (2009).
18. R. F. Wynn, J. E. Wraith, J. Mercer, A. O'Meara, K. Tylee, M. Thornley, H. J. Church, B. W. Bigger, Improved metabolic correction in patients with lysosomal storage disease treated with hematopoietic stem cell transplant compared with enzyme replacement therapy. J Pediatr 154, 609-611 (2009).
19. A. Biffi, E. Montini, L. Lorioli, M. Cesani, F. Fumagalli, T. Plati, C. Baldoli, S. Martino, A. Calabria, S. Canale, F. Benedicenti, G. Vallanti, L. Biasco, S. Leo, N. Kabbara, G. Zanetti, W. B. Rizzo, N. A. Mehta, M. P. Cicalese, M. Casiraghi, J. J. Boelens, U. Del Carro, D. J. Dow, M. Schmidt, A. Assanelli, V. Neduva, C. Di Serio, E. Stupka, J. Gardner, C. von Kalle, C. Bordignon, F. Ciceri, A. Rovelli, M. G. Roncarolo, A. Aiuti, M. Sessa, L. Naldini, Lentiviral hematopoietic stem cell gene therapy benefits metachromatic leukodystrophy. Science 341, 1233158 (2013).
20. F. Eichler, C. Duncan, P. L. Musolino, P. J. Orchard, S. De Oliveira, A. J. Thrasher, M. Armant, C. Dansereau, T. C. Lund, W. P. Miller, G. V. Raymond, R. Sankar, A. J. Shah, C. Sevin, H. B. Gaspar, P. Gissen, H. Amartino, D. Bratkovic, N. J. C. Smith, A. M. Paker, E. Shamir, T. O'Meara, D. Davidson, P. Aubourg, D. A. Williams, Hematopoietic Stem-Cell Gene Therapy for Cerebral Adrenoleukodystrophy. N Engl J Med 377, 1630-1638 (2017).
21. X. Cao, E W Shores, J Hu-Li, M R Anver, B L Kelsall, S M Russell, J Drago, M Noguchi, A Grinberg, E T Bloom, W E Paul, S I Katz, P E Love, W J Leonard, Defective lymphoid development in mice lacking expression of the common cytokine receptor gamma chain. Immunity, 2, 223-238 (1995)

Sequence Listing
CMV promoter sequence 1:
SEQ ID NO: 1
GCAAATGGGCGGTAGGCGTGTACGGTGGGAGGTTTATATA
AGCAGAGCTCGTTTAGTGAACCGTCAGATC,
CMV promoter sequence 2:
SEQ ID NO: 2
CATTGAT TATTGACTAG TTATTAATAG
TAATCAATTA CGGGGTCATT
AGTTCATAGC CCATATATGG AGTTCCGCGT TACATAACTT
ACGGTAAATG GCCCGCCTGG CTGACCGCCC AACGACCCCC
GCCCATTGAC GTCAATAATGACGTATGTTC CCATAGTAAC
GCCAATAGGG ACTTTCCATT GACGTCAATG GGTGGAGTAT
TTACGGTAAA CTGCCCACTT GGCAGTACAT CAAGTGTATC
ATATGCCAAG TACGCCCCCTATTGACGTCA ATGACGGTAA
ATGGCCCGCC TGGCATTATG CCCAGTACAT GACCTTATGG
GACTTTCCTA CTTGGCAGTA CATCTACGTA TTAGTCATCG
CTATTACCAT GGT,
MNDU3 promoter
SEQ ID NO: 3
CGATTAGTCCAATTTGTTAAAGACAGGATATCAGT
GGTCCAGGCTCTAGTTTTGACTCAACAATATCACC
AGCTGAAGCCTATAGAGTACGAGCCATAGATAGAA
TAAAAGATTTTATTTAGTCTCCAGAAAAAGGGGGG
AATGAAAGACCCCACCTGTAGGTTTGGCAAGCTAG
GATCAAGGTTAGGAACAGAGAGACAGCAGAATATG
GGCCAAACAGGATATCTGTGGTAAGCAGTTCCTGC
CCCGGCTCAGGGCCAAGAACAGTTGGAACAGCAGA
ATATGGGCCAAACAGGATATCTGTGGTAAGCAGTT
CCTGCCCCGGCTCAGGGCCAAGAACAGATGGTCCC
CAGATGCGGTCCCGCCCTCAGCAGTTTCTAGAGAA
CCATCAGATGTTTCCAGGGTGCCCCAAGGACCTGA
AATGACCCTGTGCCTTATTTGAACTAACCAATCAG
TTCGCTTCTCGCTTCTGTTCGCGCGCTTCTGCTCC
CCGAGCTCAATAAAAGAGCCCACAACCCCTCACTC
GGCGCGATCTAGATCTCGAATCGAATTCGCCCTT,
PGK promoter
SEQ ID NO: 4
TACCGGGTAGGGGAGGCGCTTTTCCCAAGGCAGTC
TGGAGCATGCGCTTTAGCAGCCCCGCTGGCACTTG
GCGCTACACAAGTGGCCTCTGGCCTCGCACACATT
CCACATCCACCGGTAGGCGCCAACCGGCTCCGTTC
TTTGGTGGCCCCTTCGCGCCACCTTCTACTCCTCC
CCTAGTCAGGAAGTTCCCCCCCGCCCCGCAGCTCG
CGTCGTGCAGGACGTGACAAATGGAAGTAGCACGT
CTCACTAGTCTCGTGCAGATGGACAGCACCGCTGA
GCAATGGAAGCGGGTAGGCCTTTGGGGCAGCGGCC
AATAGCAGCTTTGCTCCTTCGCTTTCTGGGCTCAG
AGGCTGGGAAGGGGTGGGTCCGGGGGCGGGCTCAG
GGGCGGGCTCAGGGGCGGGGCGGGCGCCCGAAGGT
CCTCCGGAGGCCCGGCATTCTGCACGCTTCAAAAG
CGCACGTCTGCCGCGCTGTTCTCCTCTTCCTCATC
TCCGGGCCTTTCGACCATCTA,
MNDU promoter sequence:
SEQ ID NO: 5
AACTAACCAATCAGTTCGCTTCTCGCTTCTGTTCG
CGCGCTTCTGCTCCCCGAGCTCTATATAAGCAGAG
CTCGTTTAGTGAACCGTCAGATCGCCTGGAGACGC
CATCCACGCTGTTTTGACCTCCATAGAAGACACCG
ACTCTAGAGGATC,
EF1 alpha promoter sequence:
SEQ ID NO: 6
AAGGATCTGCGATCGCTCCGGTGCCCGTCAGTGGG
CAGAGCGCACATCGCCCACAGTCCCCGAGAAGTTG
GGGGGAGGGGTCGGCAATTGAACGGGTGCCTAGAG
AAGGTGGCGCGGGGTAAACTGGGAAAGTGATGTCG
TGTACTGGCTCCGCCTTTTTCCCGAGGGTGGGGGA
GAACCGTATATAAGTGCAGTAGTCGCCGTGAACGT
TCTTTTTCGCAACGGGTTTGCCGCCAGAACACAGC
TGAAGCTTCGAGGGGCTCGCATCTCTCCTTCACGC
GCCCGCCGCCCTACCTGAGGCCGCCATCCACGCCG
GTTGAGTCGCGTTCTGCCGCCTCCCGCCTGTGGTG
CCTCCTGAACTGCGTCCGCCGTCTAGGTAAGTTTA
AAGCTCAGGTCGAGACCGGGCCTTTGTCCGGCGCT
CCCTTGGAGCCTACCTAGACTCAGCCGGCTCTCCA
CGCTTTGCCTGACCCTGCTTGCTCAACTCTACGTC
TTTGTTTCGTTTTCTGTTCTGCGCCGTTACAGATC
CAAGCTGTGACCGGCGCCTAC,
WT human Ube3a isoform 1
SEQ ID NO: 7
1-atgaagcgagcagc tgcaaagcat ctaatagaac
gctactacca ccagttaact gagggctgtg
gaaatgaagc ctgcacgaat gagttttgtg
cttcctgtcc aacttttctt cgtatggata
ataatgcagc agctattaaa gccctcgagc
tttataagat taatgcaaaa ctctgtgatc
ctcatccctc caagaaagga gcaagctcag
cttaccttga gaactcgaaa ggtgccccca
acaactcctg ctctgagata aaaatgaaca
agaaaggcgc tagaattgat tttaaagatg
tgacttactt aacagaagag aaggtatatg
aaattcttga attatgtaga gaaagagagg
attattcccc tttaatccgt gttattggaa
gagttttttc tagtgctgag gcattggtac
agagcttccg gaaagttaaa caacacacca
aggaagaact gaaatctctt caagcaaaag
atgaagacaa agatgaagat gaaaaggaaa
aagctgcatg ttctgctgct gctatggaag
aagactcaga agcatcttcc tcaaggatag
gtgatagctc acagggagac aacaatttgc
aaaaattagg ccctgatgat gtgtctgtgg
atattgatgc cattagaagg gtctacacca
gattgctctc taatgaaaaa attgaaactg
cctttctcaa tgcacttgta tatttgtcac
ctaacgtgga atgtgacttg acgtatcaca
atgtatactc tcgagatcct aattatctga
atttgttcat tatcgtaatg gagaatagaa
atctccacag tcctgaatat ctggaaatgg
ctttgccatt attttgcaaa gcgatgagca
agctacccct tgcagcccaa ggaaaactga
tcagactgtg gtctaaatac aatgcagacc
agattcggag aatgatggag acatttcagc
aacttattac ttataaagtc ataagcaatg
aatttaacag tcgaaatcta gtgaatgatg
atgatgccat tgttgctgct tcgaagtgct
tgaaaatggt ttactatgca aatgtagtgg
gaggggaagt ggacacaaat cacaatgaag
aagatgatga agagcccatc cctgagtcca
gcgagctgac acttcaggaa cttttgggag
aagaaagaag aaacaagaaa ggtcctcgag
tggaccccct ggaaactgaa cttggtgtta
aaaccctgga ttgtcgaaaa ccacttatcc
cifitgaaga gtttattaat gaaccactga
atgaggttct agaaatggat aaagattata
ctifittcaa agtagaaaca gagaacaaat
tctcttttat gacatgtccc tttatattga
atgctgtcac aaagaatttg ggattatatt
atgacaatag aattcgcatg tacagtgaac
gaagaatcac tgttctctac agcttagttc
aaggacagca gttgaatcca tatttgagac
tcaaagttag acgtgaccat atcatagatg
atgcacttgt ccggctagag atgatcgcta
tggaaaatcc tgcagacttg aagaagcagt
tgtatgtgga atttgaagga gaacaaggag
ttgatgaggg aggtgtttcc aaagaatttt
ttcagctggt tgtggaggaa atcttcaatc
cagatattgg tatgttcaca tacgatgaat
ctacaaaatt gttttggttt aatccatctt
cttttgaaac tgagggtcag tttactctga
ttggcatagt actgggtctg gctatttaca
ataactgtat actggatgta cattttccca
tggttgtcta caggaagcta atggggaaaa
aaggaacttt tcgtgacttg ggagactctc
acccagttct atatcagagt ttaaaagatt
tattggagta tgaagggaat gtggaagatg
acatgatgat cactttccag atatcacaga
cagatctttt tggtaaccca atgatgtatg
atctaaagga aaatggtgat aaaattccaa
ttacaaatga aaacaggaag gaatttgtca
atctttattc tgactacatt ctcaataaat
cagtagaaaa acagttcaag gcttttcgga
gaggttttca tatggtgacc aatgaatctc
ccttaaagta cttattcaga ccagaagaaa
ttgaattgct tatatgtgga agccggaatc
tagatttcca agcactagaa gaaactacag
aatatgacgg tggctatacc agggactctg
ttctgattag ggagttctgg gaaatcgttc
attcatttac agatgaacag aaaagactct
tcttgcagtt tacaacgggc acagacagag
cacctgtggg aggactagga aaattaaaga
tgattatagc caaaaatggc ccagacacag
aaaggttacc tacatctcat acttgcttta
atgtgctttt acttccggaa tactcaagca
aagaaaaact taaagagaga ttgttgaagg
ccatcacgta tgccaaagga tttggcatgc
tgtaa-2559,
WT human Ube3a isoform 1 aa sequence
SEQ ID NO: 8
1-MKRAAAKHLIERYYHQLTEGCGNEACTNEFCAS
CPTFLRMDNNAAAIKALELYKINAKLCDPHPSKKG
ASSAYLENSKGAPNNSCSEIKMNKKGARIDFKDVT
YLTEEKVYEILELCREREDYSPLIRVIGRVFSSAE
ALVQSFRKVKQHTKEELKSLQAKDEDKDEDEKEKA
ACSAAAMEEDSEASSSRIGDSSQGDNNLQKLGPDD
VSVDIDAIRRVYTRLLSNEKIETAFLNALVYLSPN
VECDLTYHNVYSRDPNYLNLFIIVMENRNLHSPEY
LEMALPLFCKAMSKLPLAAQGKLIRLWSKYNADQI
RRMMETFQQLITYKVISNEFNSRNLVNDDDAIVAA
SKCLKMVYYANVVGGEVDTNHNEEDDEEPIPESSE
LTLQELLGEERRNKKGPRVDPLETELGVKTLDCRK
PLIPFEEFINEPLNEVLEMDKDYTFFKVETENKFS
FMTCPFILNAVTKNLGLYYDNRIRMYSERRITVLY
SLVQGQQLNPYLRLKVRRDHIIDDALVRLEMIAME
NPADLKKQLYVEFEGEQGVDEGGVSKEFFQLVVEE
IFNPDIG1VIFTYDESTKLFWFNPSSFETEGQFTL
IGIVLGLAIYNNCILDVHFPMVVYRKLMGKKGTFR
DLGDSHPVLYQSLKDLLEYEGNVEDDMMITFQISQ
TDLFGNPMMYDLKENGDKIPITNENRKEFVNLYSD
YILNKSVEKQFKAFRRGFHMVTNESPLKYLFRPEE
IELLICGSRNLDFQALEETTEYDGGYTRDSVLIRE
FWEIVHSFTDEQKRLFLQFTTGTDRAPVGGLGKLK
MIIAKNGPDTERLPTSHTCFNVLLLPEYSSKEKLK
ERLLKAITYAKGFGML-852,
WT human Ube3a isoform 2
SEQ ID NO: 9
1-atggagaagctg
caccagtgtt attggaaatc aggagaacct
cagtctgacg acattgaagc tagccgaatg
aagcgagcag ctgcaaagca tctaatagaa
cgctactacc accagttaac tgagggctgt
ggaaatgaag cctgcacgaa tgagttttgt
gcttcctgtc caacttttct tcgtatggat
aataatgcag cagctattaa agccctcgag
ctttataaga ttaatgcaaa actctgtgat
cctcatccct ccaagaaagg agcaagctca
gcttaccttg agaactcgaa aggtgccccc
aacaactcct gctctgagat aaaaatgaac
aagaaaggcg ctagaattga ttttaaagat
gtgacttact taacagaaga gaaggtatat
gaaattcttg aattatgtag agaaagagag
gattattccc ctttaatccg tgttattgga
agagtttttt ctagtgctga ggcattggta
cagagcttcc ggaaagttaa acaacacacc
aaggaagaac tgaaatctct tcaagcaaaa
gatgaagaca aagatgaaga tgaaaaggaa
aaagctgcat gttctgctgc tgctatggaa
gaagactcag aagcatcttc ctcaaggata
ggtgatagct cacagggaga caacaatttg
caaaaattag gccctgatga tgtgtctgtg
gatattgatg ccattagaag ggtctacacc
agattgctct ctaatgaaaa aattgaaact
gcctttctca atgcacttgt atatttgtca
cctaacgtgg aatgtgactt gacgtatcac
aatgtatact ctcgagatcc taattatctg
aatttgttca ttatcgtaat ggagaataga
aatctccaca gtcctgaata tctggaaatg
gctttgccat tattttgcaa agcgatgagc
aagctacccc ttgcagccca aggaaaactg
atcagactgt ggtctaaata caatgcagac
cagattcgga gaatgatgga gacatttcag
caacttatta cttataaagt cataagcaat
gaatttaaca gtcgaaatct agtgaatgat
gatgatgcca ttgttgctgc ttcgaagtgc
ttgaaaatgg tttactatgc aaatgtagtg
ggaggggaag tggacacaaa tcacaatgaa
gaagatgatg aagagcccat ccctgagtcc
agcgagctga cacttcagga acttttggga
gaagaaagaa gaaacaagaa aggtcctcga
gtggaccccc tggaaactga acttggtgtt
aaaaccctgg attgtcgaaa accacttatc
ccttttgaag agtttattaa tgaaccactg
aatgaggttc tagaaatgga taaagattat
acttttttca aagtagaaac agagaacaaa
ttctctttta tgacatgtcc ctttatattg
aatgctgtca caaagaattt gggattatat
tatgacaata gaattcgcat gtacagtgaa
cgaagaatca ctgttctcta cagcttagtt
caaggacagc agttgaatcc atatttgaga
ctcaaagtta gacgtgacca tatcatagat
gatgcacttg tccggctaga gatgatcgct
atggaaaatc ctgcagactt gaagaagcag
ttgtatgtgg aatttgaagg agaacaagga
gttgatgagg gaggtgtttc caaagaattt
tttcagctgg ttgtggagga aatcttcaat
ccagatattg gtatgttcac atacgatgaa
tctacaaaat tgttttggtt taatccatct
tcttttgaaa ctgagggtca gtttactctg
attggcatag tactgggtct ggctatttac
aataactgta tactggatgt acattttccc
atggttgtct acaggaagct aatggggaaa
aaaggaactt ttcgtgactt gggagactct
cacccagttc tatatcagag tttaaaagat
ttattggagt atgaagggaa tgtggaagat
gacatgatga tcactttcca gatatcacag
acagatcttt ttggtaaccc aatgatgtat
gatctaaagg aaaatggtga taaaattcca
attacaaatg aaaacaggaa ggaatttgtc
aatctttatt ctgactacat tctcaataaa
tcagtagaaa aacagttcaa ggcttttcgg
agaggttttc atatggtgac caatgaatct
cccttaaagt acttattcag accagaagaa
attgaattgc ttatatgtgg aagccggaat
ctagatttcc aagcactaga agaaactaca
gaatatgacg gtggctatac cagggactct
gttctgatta gggagttctg ggaaatcgtt
cattcattta cagatgaaca gaaaagactc
ttcttgcagt ttacaacggg cacagacaga
gcacctgtgg gaggactagg aaaattaaag
atgattatag ccaaaaatgg cccagacaca
gaaaggttac ctacatctca tacttgcttt
aatgtgcttt tacttccgga atactcaagc
aaagaaaaac ttaaagagag attgttgaag
gccatcacgt atgccaaagg atttggcatg
ctgtaa-2628,
WT human Ube3a isoform 2 aa sequence
SEQ ID NO: 10
1-MEKLHQCYWKSGEPQSDDIEASRMKRAAAKHLI
ERYYHQLTEGCGNEACTNEFCASCPTFLRMDNNAA
AIKALELYKINAKLCDPHPSKKGASSAYLENSKGA
PNNSCSEIKMNKKGARIDFKDVTYLTEEKVYEILE
LCREREDYSPLIRVIGRVFSSAEALVQSFRKVKQH
TKEELKSLQAKDEDKDEDEKEKAACSAAAMEEDSE
ASSSRIGDSSQGDNNLQKLGPDDVSVDIDAIRRVY
TRLLSNEKIETAFLNALVYLSPNVECDLTYHNVYS
RDPNYLNLFIIVMENRNLHSPEYLEMALPLFCKAM
SKLPLAAQGKLIRLWSKYNADQIRRMMETFQQLIT
YKVISNEFNSRNLVNDDDAIVAASKCLKMVYYANV
VGGEVDTNHNEEDDEEPIPESSELTLQELLGEERR
NKKGPRVDPLETELGVKTLDCRKPLIPFEEFINEP
LNEVLEMDKDYTFFKVETENKFSFMTCPFILNAVT
KNLGLYYDNRIRMYSERRITVLYSLVQGQQLNPYL
RLKVRRDHIIDDALVRLEMIAMENPADLKKQLYVE
FEGEQGVDEGGVSKEFFQLVVEEIFNPDIGMFTYD
ESTKLFWFNPSSFETEGQFTLIGIVLGLAIYNNCI
LDVHFPMVVYRKLMGKKGTFRDLGDSHPVLYQSLK
DLLEYEGNVEDDMMITFQISQTDLFGNPMMYDLKE
NGDKIPITNENRKEFVNLYSDYILNKSVEKQFKAF
RRGFHMVTNESPLKYLFRPEEIELLICGSRNLDFQ
ALEETTEYDGGYTRDSVLIREFWEIVHSFTDEQKR
LFLQFTTGTDRAPVGGLGKLKMIIAKNGPDTERLP
TSHTCFNVLLLPEYSSKEKLKERLLKAITYAKGFG
ML-875,
WT human Ube3a isoform 3
SEQ ID NO: 11
1-atggccaca
gcttgtaaaa gatcaggaga acctcagtct
gacgacattg aagctagccg aatgaagcga
gcagctgcaa agcatctaat agaacgctac
taccaccagt taactgaggg ctgtggaaat
gaagcctgca cgaatgagtt ttgtgcttcc
tgtccaactt ttcttcgtat ggataataat
gcagcagcta ttaaagccct cgagctttat
aagattaatg caaaactctg tgatcctcat
ccctccaaga aaggagcaag ctcagcttac
cttgagaact cgaaaggtgc ccccaacaac
tcctgctctg agataaaaat gaacaagaaa
ggcgctagaa ttgattttaa agatgtgact
tacttaacag aagagaaggt atatgaaatt
cttgaattat gtagagaaag agaggattat
tcccctttaa tccgtgttat tggaagagtt
ttttctagtg ctgaggcatt ggtacagagc
ttccggaaag ttaaacaaca caccaaggaa
gaactgaaat ctcttcaagc aaaagatgaa
gacaaagatg aagatgaaaa ggaaaaagct
gcatgttctg ctgctgctat ggaagaagac
tcagaagcat cttcctcaag gataggtgat
agctcacagg gagacaacaa tttgcaaaaa
ttaggccctg atgatgtgtc tgtggatatt
gatgccatta gaagggtcta caccagattg
ctctctaatg aaaaaattga aactgccttt
ctcaatgcac ttgtatattt gtcacctaac
gtggaatgtg acttgacgta tcacaatgta
tactctcgag atcctaatta tctgaatttg
ttcattatcg taatggagaa tagaaatctc
cacagtcctg aatatctgga aatggctttg
ccattatttt gcaaagcgat gagcaagcta
ccccttgcag cccaaggaaa actgatcaga
ctgtggtcta aatacaatgc agaccagatt
cggagaatga tggagacatt tcagcaactt
attacttata aagtcataag caatgaattt
aacagtcgaa atctagtgaa tgatgatgat
gccattgttg ctgcttcgaa gtgcttgaaa
atggtttact atgcaaatgt agtgggaggg
gaagtggaca caaatcacaa tgaagaagat
gatgaagagc ccatccctga gtccagcgag
ctgacacttc aggaactttt gggagaagaa
agaagaaaca agaaaggtcc tcgagtggac
cccctggaaa ctgaacttgg tgttaaaacc
ctggattgtc gaaaaccact tatccctttt
gaagagttta ttaatgaacc actgaatgag
gttctagaaa tggataaaga ttatactttt
ttcaaagtag aaacagagaa caaattctct
tttatgacat gtccctttat attgaatgct
gtcacaaaga atttgggatt atattatgac
aatagaattc gcatgtacag tgaacgaaga
atcactgttc tctacagctt agttcaagga
cagcagttga atccatattt gagactcaaa
gttagacgtg accatatcat agatgatgca
cttgtccggc tagagatgat cgctatggaa
aatcctgcag acttgaagaa gcagttgtat
gtggaatttg aaggagaaca aggagttgat
gagggaggtg tttccaaaga attttttcag
ctggttgtgg aggaaatctt caatccagat
attggtatgt tcacatacga tgaatctaca
aaattgtttt ggtttaatcc atcttctttt
gaaactgagg gtcagtttac tctgattggc
atagtactgg gtctggctat ttacaataac
tgtatactgg atgtacattt tcccatggtt
gtctacagga agctaatggg gaaaaaagga
acttttcgtg acttgggaga ctctcaccca
gttctatatc agagtttaaa agatttattg
gagtatgaag ggaatgtgga agatgacatg
atgatcactt tccagatatc acagacagat
ctttttggta acccaatgat gtatgatcta
aaggaaaatg gtgataaaat tccaattaca
aatgaaaaca ggaaggaatt tgtcaatctt
tattctgact acattctcaa taaatcagta
gaaaaacagt tcaaggcttt tcggagaggt
tttcatatgg tgaccaatga atctccctta
aagtacttat tcagaccaga agaaattgaa
ttgcttatat gtggaagccg gaatctagat
ttccaagcac tagaagaaac tacagaatat
gacggtggct ataccaggga ctctgttctg
attagggagt tctgggaaat cgttcattca
tttacagatg aacagaaaag actcttcttg
cagtttacaa cgggcacaga cagagcacct
gtgggaggac taggaaaatt aaagatgatt
atagccaaaa atggcccaga cacagaaagg
ttacctacat ctcatacttg ctttaatgtg
cttttacttc cggaatactc aagcaaagaa
aaacttaaag agagattgtt gaaggccatc
acgtatgcca aaggatttgg catgctgtaa-2619
WT human Ube3a isoform 3 aa sequence
SEQ ID NO: 12
1-MATACKRSGEPQSDDIEASRMKRAAAKHLIERY
YHQLTEGCGNEACTNEFCASCPTFLRMDNNAAAIK
ALELYKINAKLCDPHPSKKGASSAYLENSKGAPNN
SCSEIKMNKKGARIDFKDVTYLTEEKVYEILELCR
EREDYSPLIRVIGRVFSSAEALVQSFRKVKQHTKE
ELKSLQAKDEDKDEDEKEKAACSAAAMEEDSEASS
SRIGDSSQGDNNLQKLGPDDVSVDIDAIRRVYTRL
LSNEKIETAFLNALVYLSPNVECDLTYHNVYSRDP
NYLNLFIIVMENRNLHSPEYLEMALPLFCKAMSKL
PLAAQGKLIRLWSKYNADQIRRMMETFQQLITYKV
ISNEFNSRNLVNDDDAIVAASKCLKMVYYANVVGG
EVDTNHNEEDDEEPIPESSELTLQELLGEERRNKK
GPRVDPLETELGVKTLDCRKPLIPFEEFINEPLNE
VLEMDKDYTFFKVETENKFSFMTCPFILNAVTKNL
GLYYDNRIRMYSERRITVLYSLVQGQQLNPYLRLK
VRRDHIIDDALVRLEMIAMENPADLKKQLYVEFEG
EQGVDEGGVSKEFFQLVVEEIFNPDIGMFTYDEST
KLFWFNPSSFETEGQFTLIGIVLGLAIYNNCILDV
HFPMVVYRKLMGKKGTFRDLGDSHPVLYQSLKDLL
EYEGNVEDDMMITFQISQTDLFGNPMMYDLKENGD
KIPITNENRKEFVNLYSDYILNKSVEKQFKAFRRG
FHMVTNESPLKYLFRPEEIELLICGSRNLDFQALE
ETTEYDGGYTRDSVLIREFWEIVHSFTDEQKRLFL
QFTTGTDRAPVGGLGKLKMIIAKNGPDTERLPTSH
TCFNVLLLPEYSSKEKLKERLLKAITYAKGFGML-872,

### Modified Human Isoform #1

Nucleotide positions 190, 293, 310, 661, 662, 1066, 1067, 1771, 1773, 1870, 1871, 2413, 2414, 2417, and 2418
AA positions 64, 98, 104, 221, 356, 591, 624, 805, and 806
SEQ ID NO: 13, Human Ube3a isoform 1 with 8×N-glycan sites (bolded) and IL2 secretion signal (underlined). Bold and capitalized font provides examples of mutations to create a glycosylation site.

1-atgtacaggatgcaa ctcctgtctt gcattgcact
aagtcttgca cttgtcacaa acagt atgaagcga
gcagc tgcaaagcat ctaatagaacgctactacca
ccagttaact gagggctgtg gaaatgaagc
ctgcacgaat gagttttgtg cttcctgtcc
aacttttctt cgtatggata ataatAcagc
agctattaaa gccctcgagc tttataagat
taatgcaaaa ctctgtgatc ctcatccctc
caagaaagga gcaagctcag cttaccttga
gaactcgaCa ggtgccccca acaacAcctg
ctctgagata aaaatgaaca agaaaggcgc
tagaattgat tttaaagatg tgacttactt
aacagaagag aaggtatatg aaattcttga
attatgtaga gaaagagagg attattcccc
tttaatccgt gttattggaa gagttttttc
tagtgctgag gcattggtac agagcttccg
gaaagttaaa caacacacca aggaagaact
gaaatctctt caagcaaaag atgaagacaa
agatgaagat gaaaaggaaa aagctgcatg
ttctgctgct gctatggaag aagactcaga
agcatcttcc tcaaggatag gtgatagctc
acagggagac aacaatACgc aaaaattagg
ccctgatgat gtgtctgtgg atattgatgc
cattagaagg gtctacacca gattgctctc
taatgaaaaa attgaaactg cctttctcaa
tgcacttgta tatttgtcac ctaacgtgga
atgtgacttg acgtatcaca atgtatactc
tcgagatcct aattatctga atttgttcat
tatcgtaatg gagaatagaa atctccacag
tcctgaatat ctggaaatgg ctttgccatt
attttgcaaa gcgatgagca agctacccct
tgcagcccaa ggaaaactga tcagactgtg
gtctaaatac aatgcagacc agattcggag
aatgatggag acatttcagc aacttattac
ttataaagtc ataagcaatg aatttaacag
tACaaatcta gtgaatgatg atgatgccat
tgttgctgct tcgaagtgct tgaaaatggt
ttactatgca aatgtagtgg gaggggaagt
ggacacaaat cacaatgaag aagatgatga
agagcccatc cctgagtcca gcgagctgac
acttcaggaa cttttgggag aagaaagaag
aaacaagaaa ggtcctcgag tggaccccct
ggaaactgaa cttggtgtta aaaccctgga
ttgtcgaaaa ccacttatcc cttttgaaga
gtttattaat gaaccactga atgaggttct
agaaatggat aaagattata cttttttcaa
agtagaaaca gagaacaaat tctcttttat
gacatgtccc tttatattga atgctgtcac
aaagaatttg ggattatatt atgacaatag
aattcgcatg tacagtgaac gaagaatcac
tgttctctac agcttagttc aaggacagca
gttgaatcca tatttgagac tcaaagttag
acgtgaccat atcatagatg atgcacttgt
ccggctagag atgatcgcta tggaaaatcc
tgcagacttg aagaagcagt tgtatgtgga
atttgaagga gaacaaggag ttgatgaggg
aggtgtttcc aaagaatttt ttcagctggt
tgtggaggaa atcttcaatc cagatattgg
tatgttcaca tacgatAaCt ctacaaaatt
gttttggttt aatccatctt cttttgaaac
tgagggtcag tttactctga ttggcatagt
actgggtctg gctatttaca ataacACtat
actggatgta cattttccca tggttgtcta
caggaagcta atggggaaaa aaggaacttt
tcgtgacttg ggagactctc acccagttct
atatcagagt ttaaaagatt tattggagta
tgaagggaat gtggaagatg acatgatgat
cactttccag atatcacaga cagatctttt
tggtaaccca atgatgtatg atctaaagga
aaatggtgat aaaattccaa ttacaaatga
aaacaggaag gaatttgtca atctttattc
tgactacatt ctcaataaat cagtagaaaa
acagttcaag gcttttcgga gaggttttca
tatggtgacc aatgaatctc ccttaaagta
cttattcaga ccagaagaaa ttgaattgct
tatatgtgga agccggaatc tagatttcca
agcactagaa gaaactacag aatatgacgg
tggctatacc agggactctg ttctgattag
ggagttctgg gaaatcgttc attcatttac
agatgaacag aaaagactct tcttgcagAA
taATacgggc acagacagag cacctgtggg
aggactagga aaattaaaga tgattatagc
caaaaatggc ccagacacag aaaggttacc
tacatctcat acttgcttta atgtgctttt
acttccggaa tactcaagca aagaaaaact
taaagagaga ttgttgaagg ccatcacgta
tgccaaagga tttggcatgc tgtaa-2619

SEQ ID NO: 14, Human Ube3a isoform 1 with 8×N-glycan sites (bolded) and IL2 secretion signal (underlined-first 20 amino acids) aa sequence. Italics are mutated sites for glycosylation.

1-MYRMQLLSCIALSLALVTNSMKRAAAKHLIERY
YHQLTEGCGNEACTNEFCASCPTFLRMDNN [symbol] AAIK
ALELYKINAKLCDPHPSKKGASSAYLENS [symbol] GAPNN [symbol]
CSEIKMNKKGARIDFKDVTYLTEEKVYEILELCR
EREDYSPLIRVIGRVFSSAEALVQSFRKVKQHTKE
ELKSLQAKDEDKDEDEKEKAACSAAAMEEDSEASS
SRIGDSSQGDNN [symbol] QKLGPDDVSVDIDAIRRVYTRL
LSNEKIETAFLNALVYLSPNVECDLTYHNVYSRDP
NYLNLFIIVMENRNLHSPEYLEMALPLFCKAMSKL
PLAAQGKLIRLWSKYNADQIRRMMETFQQLITYKV
ISNEFNS [symbol] NLVNDDDAIVAASKCLKMVYYANVVGG
EVDTNHNEEDDEEPIPESSELTLQELLGEERRNKK
GPRVDPLETELGVKTLDCRKPLIPFEEFINEPLNE
VLEMDKDYTFFKVETENKFSFMTCPFILNAVTKNL
GLYYDNRIRMYSERRITVLYSLVQGQQLNPYLRLK
VRRDHIIDDALVRLEMIAMENPADLKKQLYVEFEG
EQGVDEGGVSKEFFQLVVEEIFNPDIGMFTYDNST artwork
KLFWFNPSSFETEGQFTLIGIVLGLAIYNN [symbol] ILDV
HFPMVVYRKLMGKKGTFRDLGDSHPVLYQSLKDLL
EYEGNVEDDMMITFQISQTDLFGNPMMYDLKENGD
KIPITNENRKEFVNLYSDYILNKSVEKQFKAFRRG
FHMVTNESPLKYLFRPEEIELLICGSRNLDFQALE
ETTEYDGGYTRDSVLIREFWEIVHSFTDEQKRLFL
Q [symbol] TGTDRAPVGGLGKLKMIIAKNGPDTERLPTSH
TCFNVLLLPEYSSKEKLKERLLKAITYAKGF
GML-872

### Modified Human Isoform #2

Nucleotide positions 259, 362, 379, 730, 731, 1135, 1136, 1840, 1842, 1939, 1940, 2482, 2483, 2486, and 2487
AA positions 87, 121, 127, 244, 379, 614, 647, 828, and 829
SEQ ID NO: 15, Human Ube3a isoform 2 with 8×N-glycan sites (bolded) and IL2 secretion signal (underlined). Bold and capitalized font provides examples of mutations to create a glycosylation site.

1-atgtacaggatgcaa ctcctgtctt gcattgcact
aagtcttgca cttgtcacaa acagt atggagaagctg
caccagtgtt attggaaatc aggagaacct
cagtctgacg acattgaagc tagccgaatg
aagcgagcag ctgcaaagca tctaatagaa
cgctactacc accagttaac tgagggctgt
ggaaatgaag cctgcacgaa tgagttttgt
gcttcctgtc caacttttct tcgtatggat
aataatAcag cagctattaa agccctcgag
ctttataaga ttaatgcaaa actctgtgat
cctcatccct ccaagaaagg agcaagctca
gcttaccttg agaactcgaCaggtgccccc
aacaacAcct gctctgagat aaaaatgaac
aagaaaggcg ctagaattga ttttaaagat
gtgacttact taacagaaga gaaggtatat
gaaattcttg aattatgtag agaaagagag
gattattccc ctttaatccg tgttattgga
agagtttttt ctagtgctga ggcattggta
cagagcttcc ggaaagttaa acaacacacc
aaggaagaac tgaaatctct tcaagcaaaa
gatgaagaca aagatgaaga tgaaaaggaa
aaagctgcat gttctgctgc tgctatggaa
gaagactcag aagcatcttc ctcaaggata
ggtgatagct cacagggaga caacaatACg
caaaaattag gccctgatga tgtgtctgtg
gatattgatg ccattagaag ggtctacacc
agattgctct ctaatgaaaa aattgaaact
gcctttctca atgcacttgt atatttgtca
cctaacgtgg aatgtgactt gacgtatcac
aatgtatact ctcgagatcc taattatctg
aatttgttca ttatcgtaat ggagaataga
aatctccaca gtcctgaata tctggaaatg
gctttgccat tattttgcaa agcgatgagc
aagctacccc ttgcagccca aggaaaactg
atcagactgt ggtctaaata caatgcagac
cagattcgga gaatgatgga gacatttcag
caacttatta cttataaagt cataagcaat
gaatttaaca gtACaaatct agtgaatgat
gatgatgcca ttgttgctgc ttcgaagtgc
ttgaaaatgg tttactatgc aaatgtagtg
ggaggggaag tggacacaaa tcacaatgaa
gaagatgatg aagagcccat ccctgagtcc
agcgagctga cacttcagga acttttggga
gaagaaagaa gaaacaagaa aggtcctcga
gtggaccccc tggaaactga acttggtgtt
aaaaccctgg attgtcgaaa accacttatc
ccttttgaag agtttattaa tgaaccactg
aatgaggttc tagaaatgga taaagattat
acttttttca aagtagaaac agagaacaaa
ttctctttta tgacatgtcc ctttatattg
aatgctgtca caaagaattt gggattatat
tatgacaata gaattcgcat gtacagtgaa
cgaagaatca ctgttctcta cagcttagtt
caaggacagc agttgaatcc atatttgaga
ctcaaagtta gacgtgacca tatcatagat
gatgcacttg tccggctaga gatgatcgct
atggaaaatc ctgcagactt gaagaagcag
ttgtatgtgg aatttgaagg agaacaagga
gttgatgagg gaggtgtttc caaagaattt
tttcagctgg ttgtggagga aatcttcaat
ccagatattg gtatgttcac atacgatAaC
tctacaaaat tgttttggtt taatccatct
tcttttgaaa ctgagggtca gtttactctg
attggcatag tactgggtct ggctatttac
aataacACta tactggatgt acattttccc
atggttgtct acaggaagct aatggggaaa
aaaggaactt ttcgtgactt gggagactct
cacccagttc tatatcagag tttaaaagat
ttattggagt atgaagggaa tgtggaagat
gacatgatga tcactttcca gatatcacag
acagatcttt ttggtaaccc aatgatgtat
gatctaaagg aaaatggtga taaaattcca
attacaaatg aaaacaggaa ggaatttgtc
aatctttatt ctgactacat tctcaataaa
tcagtagaaa aacagttcaa ggcttttcgg
agaggttttc atatggtgac caatgaatct
cccttaaagt acttattcag accagaagaa
attgaattgc ttatatgtgg aagccggaat
ctagatttcc aagcactaga agaaactaca
gaatatgacg gtggctatac cagggactct
gttctgatta gggagttctg ggaaatcgtt
cattcattta cagatgaaca gaaaagactc
ttcttgcagA AtaATacggg cacagacaga
gcacctgtgg gaggactagg aaaattaaag
atgattatag ccaaaaatgg cccagacaca
gaaaggttac ctacatctca tacttgcttt
aatgtgcttt tacttccgga atactcaagc
aaagaaaaac ttaaagagag attgttgaag
gccatcacgt atgccaaagg atttggcatg
ctgtaa-2688

SEQ ID NO: 16, Human Ube3a isoform 2 with 8×N-glycan sites (bolded) and IL2 secretion signal (underlined) aa sequence

1-MYRMQLLSCIALSLALVTNSMEKLHQCYWKSGE
PQSDDIEASRMKRAAAKHLIERYYHQLTEGCGNEA
CTNEFCASCPTFLRMDNNTAAIKALELYKINAKLC
DPHPSKKGASSAYLENSTGAPNNTCSEIKMNKKGA
RIDFKDVTYLTEEKVYEILELCREREDYSPLIRVI
GRVFSSAEALVQSFRKVKQHTKEELKSLQAKDEDK
DEDEKEKAACSAAAMEEDSEASSSRIGDSSQGDNN
TQKLGPDDVSVDIDAIRRVYTRLLSNEKIETAFLN
ALVYLSPNVECDLTYHNVYSRDPNYLNLFIIVMEN
RNLHSPEYLEMALPLFCKAMSKLPLAAQGKLIRLW
SKYNADQIRRMMETFQQLITYKVISNEFNSTNLVN
DDDAIVAASKCLKMVYYANVVGGEVDTNHNEEDDE
EPIPESSELTLQELLGEERRNKKGPRVDPLETELG
VKTLDCRKPLIPFEEFINEPLNEVLEMDKDYTFFK
VETENKFSFMTCPFILNAVTKNLGLYYDNRIRMYS
ERRITVLYSLVQGQQLNPYLRLKVRRDHIIDDALV
RLEMIAMENPADLKKQLYVEFEGEQGVDEGGVSKE
FFQLVVEEIFNPDIGMFTYDNSTKLFWFNPSSFET
EGQFTLIGIVLGLAIYNNTILDVHFPMVVYRKLMG
KKGTFRDLGDSHPVLYQSLKDLLEYEGNVEDDMMI
TFQISQTDLFGNPMMYDLKENGDKEPITNENRKEF
VNLYSDYTLNKSVEKQFKAFRRGFHMVTNESPLKY
LFRPEEIELLICGSRNLDFQALEETTEYDGGYTRD
SVLIREFWEIVHSFTDEQKRLFLQNNTGTDRAPVG
GLGKLKMIIAKNGPDTERLPTSHTCFNVLLLPEYS
SKEKLKERLLKAITYAKGFGML-895

### Modified Human Isoform #3

Nucleotide positions 250, 353, 370, 721, 722, 1126, 1127, 1831, 1833, 1930, 1931, 2473, 2474, 2477, and 2478
AA positions 84, 118, 124, 241, 376, 611, 644, 825, and 826
SEQ ID NO: 17, Human Ube3a isoform 3 with 8×N-glycan sites (bolded) and IL2 secretion signal (underlined). Bold and capitalized font provides examples of mutations to create a glycosylation site.

1-atgtacaggatgcaa ctcctgtctt gcattgcact
aagtcttgca cttgtcacaa acagt
atggccaca gcttgtaaaagatcaggaga
acctcagtct gacgacattg aagctagccg
aatgaagcga gcagctgcaa agcatctaat
agaacgctac taccaccagt taactgaggg
ctgtggaaat gaagcctgca cgaatgagtt
ttgtgcttcc tgtccaactt ttcttcgtat
ggataataat Acagcagcta ttaaagccct
cgagctttat aagattaatg caaaactctg
tgatcctcat ccctccaaga aaggagcaag
ctcagcttac cttgagaact cgaCaggtgc
ccccaacaac Acctgctctg agataaaaat
gaacaagaaa ggcgctagaa ttgattttaa
agatgtgact tacttaacag aagagaaggt
atatgaaatt cttgaattat gtagagaaag
agaggattat tcccctttaa tccgtgttat
tggaagagtt ttttctagtg ctgaggcatt
ggtacagagc ttccggaaag ttaaacaaca
caccaaggaa gaactgaaat ctcttcaagc
aaaagatgaa gacaaagatg aagatgaaaa
ggaaaaagct gcatgttctg ctgctgctat
ggaagaagac tcagaagcat cttcctcaag
gataggtgat agctcacagg gagacaacaa
tACgcaaaaa ttaggccctg atgatgtgtc
tgtggatatt gatgccatta gaagggtcta
caccagattg ctctctaatg aaaaaattga
aactgccttt ctcaatgcac ttgtatattt
gtcacctaac gtggaatgtg acttgacgta
tcacaatgta tactctcgag atcctaatta
tctgaatttg ttcattatcg taatggagaa
tagaaatctc cacagtcctg aatatctgga
aatggctttg ccattatttt gcaaagcgat
gagcaagcta ccccttgcag cccaaggaaa
actgatcaga ctgtggtcta aatacaatgc
agaccagatt cggagaatga tggagacatt
tcagcaactt attacttata aagtcataag
caatgaattt aacagtACaa atctagtgaa
tgatgatgat gccattgttg ctgcttcgaa
gtgcttgaaa atggtttact atgcaaatgt
agtgggaggg gaagtggaca caaatcacaa
tgaagaagat gatgaagagc ccatccctga
gtccagcgag ctgacacttc aggaactttt
gggagaagaa agaagaaaca agaaaggtcc
tcgagtggac cccctggaaa ctgaacttgg
tgttaaaacc ctggattgtc gaaaaccact
tatccctttt gaagagttta ttaatgaacc
actgaatgag gttctagaaa tggataaaga
ttatactttt ttcaaagtag aaacagagaa
caaattctct tttatgacat gtccctttat
attgaatgct gtcacaaaga atttgggatt
atattatgac aatagaattc gcatgtacag
tgaacgaaga atcactgttc tctacagctt
agttcaagga cagcagttga atccatattt
gagactcaaa gttagacgtg accatatcat
agatgatgca cttgtccggc tagagatgat
cgctatggaa aatcctgcag acttgaagaa
gcagttgtat gtggaatttg aaggagaaca
aggagttgat gagggaggtg tttccaaaga
attttttcag ctggttgtgg aggaaatctt
caatccagat attggtatgt tcacatacga
tAaCtctaca aaattgtttt ggtttaatcc
atcttctttt gaaactgagg gtcagtttac
tctgattggc atagtactgg gtctggctat
ttacaataac ACtatactgg atgtacattt
tcccatggtt gtctacagga agctaatggg
gaaaaaagga acttttcgtg acttgggaga
ctctcaccca gttctatatc agagtttaaa
agatttattg gagtatgaag ggaatgtgga
agatgacatg atgatcactt tccagatatc
acagacagat ctttttggta acccaatgat
gtatgatcta aaggaaaatg gtgataaaat
tccaattaca aatgaaaaca ggaaggaatt
tgtcaatctt tattctgact acattctcaa
taaatcagta gaaaaacagt tcaaggcttt
tcggagaggt tttcatatgg tgaccaatga
atctccctta aagtacttat tcagaccaga
agaaattgaa ttgcttatat gtggaagccg
gaatctagat ttccaagcac tagaagaaac
tacagaatat gacggtggct ataccaggga
ctctgttctg attagggagt tctgggaaat
cgttcattca tttacagatg aacagaaaag
actcttcttg cagAAtaATa cgggcacaga
cagagcacct gtgggaggac taggaaaatt
aaagatgatt atagccaaaa atggcccaga
cacagaaagg ttacctacat ctcatacttg
ctttaatgtg cttttacttc cggaatactc
aagcaaagaa aaacttaaag agagattgtt
gaaggccatc acgtatgcca aaggatttgg
catgctgtaa-2679

SEQ ID NO: 18, Human Ube3a isoform 3 with 8×N-glycan sites (bolded) and IL2 secretion signal (underlined) aa sequence

1-MYRMQLLSCIALSLALVTNSMATACKRSGEP
QSDDIEASRMKRAAAKHLIERYYHQLTEGCGNE
ACTNEFCASCPTFLRMDNNTAAIKALELYKINA
KLCDPHPSKKGASSAYLENSTGAPNNTCSEIKM
NKKGARIDFKDVTYLTEEKVYEILELCREREDY
SPLIRVIGRVFSSAEALVQSFRKVKQHTKEELK
SLQAKDEDKDEDEKEKAACSAAAMEEDSEASSS
RIGDSSQGDNNTQKLGPDDVSVDIDAIRRVYTR
LLSNEKIETAFLNALVYLSPNVECDLTYHNVYS
RDPNYLNLFIIVMENRNLHSPEYLEMALPLFCK
AMSKLPLAAQGKLIRLWSKYNADQIRRMMETFQ
QLITYKVISNEFNSTNLVNDDDAIVAASKCLKM
VYYANVVGGEVDTNHNEEDDEEPIPESSELTLQ
ELLGEERRNKKGPRVDPLETELGVKTLDCRKPL
IPFEEFINEPLNEVLEMDKDYTFFKVETENKFS
FMTCPFILNAVTKNLGLYYDNRIRMYSERRITV
LYSLVQGQQLNPYLRLKVRRDHIIDDALVRLEM
IAMENPADLKKQLYVEFEGEQGVDEGGVSKEFF
QLVVEEIFNPDIGMFTYDNSTKLFWFNPSSFET
EGQFTLIGIVLGLAIYNNTILDVHFPMVVYRKL
MGKKGTFRDLGDSHPVLYQSLKDLLEYEGNVED
DMMITFQISQTDLFGNPMMYDLKENGDKIPITN
ENRKEFVNLYSDYILNKSVEKQFKAFRRGFHMV
TNESPLKYLFRPEEIELLICGSRNLDFQALEET
TEYDGGYTRDSVLIREFWEIVHSFTDEQKRLFL
QNNTGTDRAPVGGLGKLKMIIAKNGPDTERLPT
SHTCFNVLLLPEYSSKEKLKERLLKAITYAKGF
GML-892
WT mouse Ube3a isoform 1
SEQ ID NO: 19
1-atggccacagct tgtaaaagat caccaggaga
atcccagtct gaggacattg aagctagccg
aatgaagcga gcagctgcaa agcatctaat
agaacgctac taccatcagt taactgaggg
ctgtggaaat gaggcctgca cgaatgagtt
ttgtgcttcc tgtccaactt ttcttcgtat
ggataacaat gcagcagcta ttaaagccct
tgagctttat aaaattaatg caaaactctg
tgatcctcat ccctccaaga aaggagcaag
ctcagcttac cttgagaact caaaaggtgc
atctaacaac tcagagataa aaatgaacaa
gaaggaagga aaagatttta aagatgtgat
ttacctaact gaagagaaag tatatgaaat
ttatgaattt tgtagagaga gtgaggatta
ttccccttta attcgtgtaa ttggaagaat
attttctagt gctgaggcac tggttctgag
ctttcggaaa gtcaaacagc acacaaagga
ggaattgaaa tctcttcaag aaaaggatga
agacaaggat gaagatgaaa aggaaaaagc
tgcatgttct gctgctgcta tggaagaaga
ctcagaagca tcttcttcaa ggatgggtga
tagttcacag ggagacaaca atgtacaaaa
attaggtcct gatgatgtga ctgtggatat
tgatgctatt agaagggtct acagcagttt
gctcgctaat gaaaaattag aaactgcctt
cctgaatgca cttgtatatc tgtcacctaa
cgtggaatgt gatttgacat atcataatgt
gtatactcga gatcctaatt atctcaattt
gttcattatt gtaatggaga atagtaatct
ccacagtcct gaatatctgg aaatggcgtt
gccattattt tgcaaagcta tgtgtaagct
accccttgaa gctcaaggaa aactgattag
gctgtggtct aaatacagtg ctgaccagat
tcggagaatg atggaaacat ttcagcaact
tattacctac aaagtcataa gcaatgaatt
taatagccga aatctagtga atgatgatga
tgccattgtt gctgcttcaa agtgtttgaa
aatggtttac tatgcaaatg tagtgggagg
ggatgtggac acaaatcata atgaggaaga
tgatgaagaa cccatacctg agtccagcga
attaacactt caggagcttc tgggagatga
aagaagaaat aagaaaggtc ctcgagtgga
tccactagaa accgaacttg gcgttaaaac
tctagactgt cgaaaaccac ttatctcctt
tgaagaattc attaatgaac cactgaatga
tgttctagaa atggacaaag attatacctt
tttcaaagtt gaaacagaga acaaattctc
ttttatgaca tgtcccttta tattgaatgc
tgtcacaaag aatctgggat tatattatga
caatagaatt cgcatgtaca gtgaaagaag
aatcactgtt ctttacagcc tagttcaagg
acagcagttg aatccgtatt tgagactcaa
agtcagacgt gaccatatta tagatgatgc
actggtccgg ctagagatga ttgctatgga
aaatcctgca gacttgaaga agcagttgta
tgtggaattt gaaggagaac aaggagtaga
tgagggaggc gtttccaaag agttttttca
gttggttgtg gaggaaattt ttaatccaga
tattggtatg ttcacatatg atgaagctac
gaaattattt tggtttaatc catcttcttt
tgaaactgag ggtcagttta ctctgattgg
catagtcctg ggtctggcta tttacaataa
ttgtatactg gatgtccatt ttcccatggt
tgtatacagg aagctaatgg ggaaaaaagg
aacctttcgt gacttgggag actctcaccc
agttttatat cagagtttaa aggatttatt
ggaatatgaa gggagtgtgg aagatgatat
gatgatcact ttccagatat cacagacaga
tctttttggt aacccaatga tgtatgatct
aaaagaaaat ggtgataaaa ttccaattac
aaatgaaaac aggaaggaat ttgtcaatct
ctattcagac tacattctca ataaatctgt
agaaaaacaa ttcaaggcat ttcgcagagg
ttttcatatg gtgactaatg aatcgccctt
aaaatactta ttcagaccag aagaaattga
attgcttata tgtggaagcc ggaatctaga
tttccaggca ctagaagaaa ctacagagta
tgacggtggc tatacgaggg aatctgttgt
gattagggag ttctgggaaa ttgttcattc
gtttacagat gaacagaaaa gactctttct
gcagtttaca acaggcacag acagagcacc
tgttggagga ctaggaaaat tgaagatgat
tatagccaaa aatggcccag acacagaaag
gttacctaca tctcatactt gctttaatgt
ccttttactt ccggaatatt caagcaaaga
aaaacttaaa gagagattgt tgaaggccat
cacatatgcc aaaggatttg gcatgctgta
a-2613,
WT mouse Ube3a isoform 1 aa sequence
SEQ ID NO: 20
1-MATACKRSPGESQSEDIEASRMKRAAAKHLI
ERYYHQLTEGCGNEACTNEFCASCPTFLRMDNN
AAAIKALELYKINAKLCDPHPSKKGASSAYLEN
SKGASNNSEIKMNKKEGKDFKDVIYLTEEKVYE
IYEFCRESEDYSPLIRVIGRIFSSAEALVLSFR
KVKQHTKEELKSLQEKDEDKDEDEKEKAACSAA
AMEEDSEASSSRMGDSSQGDNNVQKLGPDDVTV
DIDAIRRVYSSLLANEKLETAFLNALVYLSPNV
ECDLTYHNVYTRDPNYLNLFIIVMENSNLHSPE
YLEMALPLFCKAMCKLPLEAQGKLIRLWSKYSA
DQIRRMMETFQQLITYKVISNEFNSRNLVNDDD
AIVAASKCLKMVYYANVVGGDVDTNHNEEDDEE
PIPESSELTLQELLGDERRNKKGPRVDPLETEL
GVKTLDCRKPLISFEEFINEPLNDVLEMDKDYT
FFKVETENKFSFMTCPFILNAVTKNLGLYYDNR
IRMYSERRITVLYSLVQGQQLNPYLRLKVRRDH
IIDDALVRLEMIAMENPADLKKQLYVEFEGEQG
VDEGGVSKEFFQLVVEEIFNPDIGMFTYDEATK
LFWFNPSSFETEGQFTLIGIVLGLAIYNNCILD
VHFPMVVYRKLMGKKGTFRDLGDSHPVLYQSLK
DLLEYEGSVEDDMMITFQISQTDLFGNPMMYDL
KENGDKIPITNENRKEFVNLYSDYILNKSVEKQ
FKAFRRGFHMVTNESPLKYLFRPEEIELLICGS
RNLDFQALEETTEYDGGYTRESVVIREFWEIVH
SFTDEQKRLFLQFTTGTDRAPVGGLGKLKMIIA
KNGPDTERLPTSHTCFNVLLLPEYSSKEKLKER
LLKAITYAKGFGML-870,
WT mouse Ube3a isoform 2
SEQ ID NO: 21
1-atggccacagc ttgtaaaaga tcaccaggag
aatcccagtc tgaggacatt gaagctagcc
gaatgaagcg agcagctgca aagcatctaa
tagaacgcta ctaccatcag ttaactgagg
gctgtggaaa tgaggcctgc acgaatgagt
tttgtgcttc ctgtccaact tttcttcgta
tggataacaa tgcagcagct attaaagccc
ttgagcttta taaaattaat gcaaaactct
gtgatcctca tccctccaag aaaggagcaa
gctcagctta ccttgagaac tcaaaaggtg
catctaacaa ctcagagata aaaatgaaca
agaaggaagg aaaagatttt aaagatgtga
tttacctaac tgaagagaaa gtatatgaaa
tttatgaatt ttgtagagag agtgaggatt
attccccttt aattcgtgta attggaagaa
tattttctag tgctgaggca ctggttctga
gctttcggaa agtcaaacag cacacaaagg
aggaattgaa atctcttcaa gaaaaggatg
aagacaagga tgaagatgaa aaggaaaaag
ctgcatgttc tgctgctgct atggaagaag
actcagaagc atcttcttca aggatgggtg
atagttcaca gggagacaac aatgtacaaa
aattaggtcc tgatgatgtg actgtggata
ttgatgctat tagaagggtc tacagcagtt
tgctcgctaa tgaaaaatta gaaactgcct
tcctgaatgc acttgtatat ctgtcaccta
acgtggaatg tgatttgaca tatcataatg
tgtatactcg agatcctaat tatctcaatt
tgttcattat tgtaatggag aatagtaatc
tccacagtcc tgaatatctg gaaatggcgt
tgccattatt ttgcaaagct atgtgtaagc
taccccttga agctcaagga aaactgatta
ggctgtggtc taaatacagt gctgaccaga
ttcggagaat gatggaaaca tttcagcaac
ttattaccta caaagtcata agcaatgaat
ttaatagccg aaatctagtg aatgatgatg
atgccattgt tgctgcttca aagtgtttga
aaatggttta ctatgcaaat gtagtgggag
gggatgtgga cacaaatcat aatgaggaag
atgatgaaga acccatacct gagtccagcg
aattaacact tcaggagctt ctgggagatg
aaagaagaaa taagaaaggt cctcgagtgg
atccactaga aaccgaactt ggcgttaaaa
ctctagactg tcgaaaacca cttatctcct
ttgaagaatt cattaatgaa ccactgaatg
atgttctaga aatggacaaa gattatacct
ttttcaaagt tgaaacagag aacaaattct
cttttatgac atgtcccttt atattgaatg
ctgtcacaaa gaatctggga ttatattatg
acaatagaat tcgcatgtac agtgaaagaa
gaatcactgt tctttacagc ctagttcaag
gacagcagtt gaatccgtat ttgagactca
aagtcagacg tgaccatatt atagatgatg
cactggtccg gctagagatg attgctatgg
aaaatcctgc agacttgaag aagcagttgt
atgtggaatt tgaaggagaa caaggagtag
atgagggagg cgtttccaaa gagttttttc
agttggttgt ggaggaaatt tttaatccag
atattggtat gttcacatat gatgaagcta
cgaaattatt ttggtttaat ccatcttctt
ttgaaactga gggtcagttt actctgattg
gcatagtcct gggtctggct atttacaata
attgtatact ggatgtccat tttcccatgg
ttgtatacag gaagctaatg gggaaaaaag
gaacctttcg tgacttggga gactctcacc
cagttttata tcagagttta aaggatttat
tggaatatga agggagtgtg gaagatgata
tgatgatcac tttccagata tcacagacag
atctttttgg taacccaatg atgtatgatc
taaaagaaaa tggtgataaa attccaatta
caaatgaaaa caggaaggaa tttgtcaatc
tctattcaga ctacattctc aataaatctg
tagaaaaaca attcaaggca tttcgcagag
gttttcatat ggtgactaat gaatcgccct
taaaatactt attcagacca gaagaaattg
aattgcttat atgtggaagc cggaatctag
atttccaggc actagaagaa actacagagt
atgacggtgg ctatacgagg gaatctgttg
tgattaggga gttctgggaa attgttcatt
cgtttacaga tgaacagaaa agactctttc
tgcagtttac aacaggcaca gacagagcac
ctgttggagg actaggaaaa ttgaagatga
ttatagccaa aaatggccca gacacagaaa
ggttacctac atctcatact tgctttaatg
tccttttact tccggaatat tcaagcaaag
aaaaacttaa agagagattg ttgaaggcca
tcacatatgc caaaggattt ggcatgctgt
aa-2613,
WT mouse Ube3a isoform 2 aa sequence
SEQ ID NO: 22
1-MATACKRSPGESQSEDIEASRMKRAAAKHLI
ERYYHQLTEGCGNEACTNEFCASCPTFLRMDNN
AAAIKALELYKINAKLCDPHPSKKGASSAYLEN
SKGASNNSEDCMNKKEGKDFKDVIYLTEEKVYE
IYEFCRESEDYSPLIRVIGRIFSSAEALVLSFR
KVKQHTKEELKSLQEKDEDKDEDEKEKAACSAA
AMEEDSEASSSRMGDSSQGDNNVQKLGPDDVTV
DIDAIRRVYSSLLANEKLETAFLNALVYLSPNV
ECDLTYHNVYTRDPNYLNLFIIVMENSNLHSPE
YLEMALPLFCKAMCKLPLEAQGKLIRLWSKYSA
DQIRRMMETFQQLITYKVISNEFNSRNLVNDDD
AIVAASKCLKMVYYANVVGGDVDTNHNEEDDEE
PIPESSELTLQELLGDERRNKKGPRVDPLETEL
GVKTLDCRKPLISFEEFINEPLNDVLEMDKDYT
FFKVETENKFSFMTCPFILNAVTKNLGLYYDNR
IRMYSERRITVLYSLVQGQQLNPYLRLKVRRDH
IIDDALVRLEMIAMENPADLKKQLYVEFEGEQG
VDEGGVSKEFFQLVVEEIFNPDIGMFTYDEATK
LFWFNPSSFETEGQFTLIGIVLGLAIYNNCILD
VHFPMVVYRKLMGKKGTFRDLGDSHPVLYQSLK
DLLEYEGSVEDDMMITFQISQTDLFGNPMMYDL
KENGDKIPITNENRKEFVNLYSDYILNKSVEKQ
FKAFRRGFHMVTNESPLKYLFRPEEIELLICGS
RNLDFQALEETTEYDGGYTRESVVIREFWEIVH
SFTDEQKRLFLQFTTGTDRAPVGGLGKLKMIIA
KNGPDTERLPTSHTCFNVLLLPEYSSKEKLKER
LLKAITYAKGFGML-870,
WT mouse Ube3a isoform 3
SEQ ID NO: 23
1-atgaa gcgagcagct gcaaagcatc taatagaacg
ctactaccat cagttaactg agggctgtgg
aaatgaggcc tgcacgaatg agttttgtgc
ttcctgtcca acttttcttc gtatggataa
caatgcagca gctattaaag cccttgagct
ttataaaatt aatgcaaaac tctgtgatcc
tcatccctcc aagaaaggag caagctcagc
ttaccttgag aactcaaaag gtgcatctaa
caactcagag ataaaaatga acaagaagga
aggaaaagat tttaaagatg tgatttacct
aactgaagag aaagtatatg aaatttatga
attttgtaga gagagtgagg attattcccc
tttaattcgt gtaattggaa gaatattttc
tagtgctgag gcactggttc tgagctttcg
gaaagtcaaa cagcacacaa aggaggaatt
gaaatctctt caagaaaagg atgaagacaa
ggatgaagat gaaaaggaaa aagctgcatg
ttctgctgct gctatggaag aagactcaga
agcatcttct tcaaggatgg gtgatagttc
acagggagac aacaatgtac aaaaattagg
tcctgatgat gtgactgtgg atattgatgc
tattagaagg gtctacagca gtttgctcgc
taatgaaaaa ttagaaactg ccttcctgaa
tgcacttgta tatctgtcac ctaacgtgga
atgtgatttg acatatcata atgtgtatac
tcgagatcct aattatctca atttgttcat
tattgtaatg gagaatagta atctccacag
tcctgaatat ctggaaatgg cgttgccatt
attttgcaaa gctatgtgta agctacccct
tgaagctcaa ggaaaactga ttaggctgtg
gtctaaatac agtgctgacc agattcggag
aatgatggaa acatttcagc aacttattac
ctacaaagtc ataagcaatg aatttaatag
ccgaaatcta gtgaatgatg atgatgccat
tgttgctgct tcaaagtgtt tgaaaatggt
ttactatgca aatgtagtgg gaggggatgt
ggacacaaat cataatgagg aagatgatga
agaacccata cctgagtcca gcgaattaac
acttcaggag cttctgggag atgaaagaag
aaataagaaa ggtcctcgag tggatccact
agaaaccgaa cttggcgtta aaactctaga
ctgtcgaaaa ccacttatct cctttgaaga
attcattaat gaaccactga atgatgttct
agaaatggac aaagattata cctttttcaa
agttgaaaca gagaacaaat tctcttttat
gacatgtccc tttatattga atgctgtcac
aaagaatctg ggattatatt atgacaatag
aattcgcatg tacagtgaaa gaagaatcac
tgttctttac agcctagttc aaggacagca
gttgaatccg tatttgagac tcaaagtcag
acgtgaccat attatagatg atgcactggt
ccggctagag atgattgcta tggaaaatcc
tgcagacttg aagaagcagt tgtatgtgga
atttgaagga gaacaaggag tagatgaggg
aggcgtttcc aaagagtttt ttcagttggt
tgtggaggaa atttttaatc cagatattgg
tatgttcaca tatgatgaag ctacgaaatt
attttggttt aatccatctt cttttgaaac
tgagggtcag tttactctga ttggcatagt
cctgggtctg gctatttaca ataattgtat
actggatgtc cattttccca tggttgtata
caggaagcta atggggaaaa aaggaacctt
tcgtgacttg ggagactctc acccagtttt
atatcagagt ttaaaggatt tattggaata
tgaagggagt gtggaagatg atatgatgat
cactttccag atatcacaga cagatctttt
tggtaaccca atgatgtatg atctaaaaga
aaatggtgat aaaattccaa ttacaaatga
aaacaggaag gaatttgtca atctctattc
agactacatt ctcaataaat ctgtagaaaa
acaattcaag gcatttcgca gaggttttca
tatggtgact aatgaatcgc ccttaaaata
cttattcaga ccagaagaaa ttgaattgct
tatatgtgga agccggaatc tagatttcca
ggcactagaa gaaactacag agtatgacgg
tggctatacg agggaatctg ttgtgattag
ggagttctgg gaaattgttc attcgtttac
agatgaacag aaaagactct ttctgcagtt
tacaacaggc acagacagag cacctgttgg
aggactagga aaattgaaga tgattatagc
caaaaatggc ccagacacag aaaggttacc
tacatctcat acttgcttta atgtcctttt
acttccggaa tattcaagca aagaaaaact
taaagagaga ttgttgaagg ccatcacata
tgccaaagga tttggcatgc tgtaa-2550,
WT mouse Ube3a isoform 3 aa sequence
SEQ ID NO: 24
1-MKRAAAKHLIERYYHQLTEGCGNEACTNEFC
ASCPTFLRMDNNAAAIKALELYKINAKLCDPHP
SKKGASSAYLENSKGASNNSEIKMNKKEGKDFK
DVIYLTEEKVYEIYEFCRESEDYSPLIRVIGRI
FSSAEALVLSFRKVKQHTKEELKSLQEKDEDKD
EDEKEKAACSAAAMEEDSEASSSRMGDSSQGDN
NVQKLGPDDVTVDIDAIRRVYSSLLANEKLETA
FLNALVYLSPNVECDLTYHNVYTRDPNYLNLFI
IVMENSNLHSPEYLEMALPLFCKAMCKLPLEAQ
GKLIRLWSKYSADQIRRMMETFQQLITYKVISN
EFNSRNLVNDDDAIVAASKCLKMVYYANVVGGD
VDTNHNEEDDEEPIPESSELTLQELLGDERRNK
KGPRVDPLETELGVKTLDCRKPLISFEEFINEP
LNDVLEMDKDYTFFKVETENKFSFMTCPFDLNA
VTKNLGLYYDNRIRMYSERRITVLYSLVQGQQL
NPYLRLKVRRDHIIDDALVRLEMIAMENPADLK
KQLYVEFEGEQGVDEGGVSKEFFQLVVEEIFNP
DIGMFTYDEATKLFWFNPSSFETEGQFTLIGIV
LGLAIYNNCILDVHFPMVVYRKLMGKKGTFRDL
GDSHPVLYQSLKDLLEYEGSVEDDMMITFQISQ
TDLFGNPMMYDLKENGDKIPITNENRKEFVNLY
SDYILNKSVEKQFKAFRRGFHMVTNESPLKYLF
RPEEIELLICGSRNLDFQALEETTEYDGGYTRE
SVVIREFWEIVHSFTDEQKRLFLQFTTGTDRAP
VGGLGKLKMIIAKNGPDTERLPTSHTCFNVLLL
PEYSSKEKLKERLLKAITYAKGFGML-849,

### Modified Mouse Isoform #1

Nucleotide positions 253, 356, 373, 715, 716, 1120, 1121, 1825, 1827, 1828, 1829, 1924, 1925, 2467, and 2468
AA positions 85, 119, 125, 239, 374, 609, 610, 642, and 823
SEQ ID NO: 25, Mouse Ube3a isoform 1 with 8×N-glycan sites (bolded) and secretion signal (underlined). Bold and capitalized font provides examples of mutations to create a glycosylation site.

1-atgtacaggatgcaa ctcctgtctt
gcattgcact aagtcttgca cttgtcacaa acagt
at ggccacagct tgtaaaagat caccaggaga
atcccagtct gaggacattg aagctagccg
aatgaagcga gcagctgcaa agcatctaat
agaacgctac taccatcagt taactgaggg
ctgtggaaat gaggcctgca cgaatgagtt
ttgtgcttcc tgtccaactt ttcttcgtat
ggataacaat Acagcagcta ttaaagccct
tgagctttat aaaattaatg caaaactctg
tgatcctcat ccctccaaga aaggagcaag
ctcagcttac cttgagaact caaCaggtgc
atctaacaac Acagagataa aaatgaacaa
gaaggaagga aaagatttta aagatgtgat
ttacctaact gaagagaaag tatatgaaat
ttatgaattt tgtagagaga gtgaggatta
ttccccttta attcgtgtaa ttggaagaat
attttctagt gctgaggcac tggttctgag
ctttcggaaa gtcaaacagc acacaaagga
ggaattgaaa tctcttcaag aaaaggatga
agacaaggat gaagatgaaa aggaaaaagc
tgcatgttct gctgctgcta tggaagaaga
ctcagaagca tcttcttcaa ggatgggtga
tagttcacag ggagacaaca atACacaaaa
attaggtcct gatgatgtga ctgtggatat
tgatgctatt agaagggtct acagcagttt
gctcgctaat gaaaaattag aaactgcctt
cctgaatgca cttgtatatc tgtcacctaa
cgtggaatgt gatttgacat atcataatgt
gtatactcga gatcctaatt atctcaattt
gttcattatt gtaatggaga atagtaatct
ccacagtcct gaatatctgg aaatggcgtt
gccattattt tgcaaagcta tgtgtaagct
accccttgaa gctcaaggaa aactgattag
gctgtggtct aaatacagtg ctgaccagat
tcggagaatg atggaaacat ttcagcaact
tattacctac aaagtcataa gcaatgaatt
taatagcACa aatctagtga atgatgatga
tgccattgtt gctgcttcaa agtgtttgaa
aatggtttac tatgcaaatg tagtgggagg
ggatgtggac acaaatcata atgaggaaga
tgatgaagaa cccatacctg agtccagcga
attaacactt caggagcttc tgggagatga
aagaagaaat aagaaaggtc ctcgagtgga
tccactagaa accgaacttg gcgttaaaac
tctagactgt cgaaaaccac ttatctcctt
tgaagaattc attaatgaac cactgaatga
tgttctagaa atggacaaag attatacctt
tttcaaagtt gaaacagaga acaaattctc
ttttatgaca tgtcccttta tattgaatgc
tgtcacaaag aatctgggat tatattatga
caatagaatt cgcatgtaca gtgaaagaag
aatcactgtt ctttacagcc tagttcaagg
acagcagttg aatccgtatt tgagactcaa
agtcagacgt gaccatatta tagatgatgc
actggtccgg ctagagatga ttgctatgga
aaatcctgca gacttgaaga agcagttgta
tgtggaattt gaaggagaac aaggagtaga
tgagggaggc gtttccaaag agttttttca
gttggttgtg gaggaaattt ttaatccaga
tattggtatg ttcacatatg atAaTAAtac
gaaattattt tggtttaatc catcttcttt
tgaaactgag ggtcagttta ctctgattgg
catagtcctg ggtctggcta tttacaataa
tACtatactg gatgtccatt ttcccatggt
tgtatacagg aagctaatgg ggaaaaaagg
aacctttcgt gacttgggag actctcaccc
agttttatat cagagtttaa aggatttatt
ggaatatgaa gggagtgtgg aagatgatat
gatgatcact ttccagatat cacagacaga
tctttttggt aacccaatga tgtatgatct
aaaagaaaat ggtgataaaa ttccaattac
aaatgaaaac aggaaggaat ttgtcaatct
ctattcagac tacattctca ataaatctgt
agaaaaacaa ttcaaggcat ttcgcagagg
ttttcatatg gtgactaatg aatcgccctt
aaaatactta ttcagaccag aagaaattga
attgcttata tgtggaagcc ggaatctaga
tttccaggca ctagaagaaa ctacagagta
tgacggtggc tatacgaggg aatctgttgt
gattagggag ttctgggaaa ttgttcattc
gtttacagat gaacagaaaa gactctttct
gcagAAtaca acaggcacag acagagcacc
tgttggagga ctaggaaaat tgaagatgat
tatagccaaa aatggcccag acacagaaag
gttacctaca tctcatactt gctttaatgt
ccttttactt ccggaatatt caagcaaaga
aaaacttaaa gagagattgt tgaaggccat
cacatatgcc aaaggatttg gcatgctgta
a-2673

SEQ ID NO: 26, Mouse Ube3a isoform 1 with 8×N-glycan sites (bolded) and secretion signal (underlined) aa sequence

1-MYRMQLLSCIALSLALVTNSMATACKRSPGESQSE
DIEASRMKRAAAKHLIERYYHQLTEGCGNEACTNE
FCASCPTFLRMDNNTAAIKALELYKINAKLCDPHP
SKKGASSAYLENSTGASNNTEIKMNKKEGKDFKDV
IYLTEEKVYEIYEFCRESEDYSPLIRVIGRIFSSA
EALVLSFRKVKQHTKEELKSLQEKDEDKDEDEKEK
AACSAAAMEEDSEASSSRMGDSSQGDNNTQKLGPD
DVTVDIDAIRRVYSSLLANEKLETAFLNALVYLSP
NVECDLTYHNVYTRDPNYLNLFIIVMENSNLHSPE
YLEMALPLFCKAMCKLPLEAQGKLIRLWSKYSADQ
IRRMMETFQQLITYKVISNEFNSTNLVNDDDAIVA
ASKCLKMVYYANVVGGDVDTNHNEEDDEEPIPESS
ELTLQELLGDERRNKKGPRVDPLETELGVKTLDCR
KPLISFEEFINEPLNDVLEMDKDYTFFKVETENKF
SFMTCPFILNAVTKNLGLYYDNRIRMYSERRITVL
YSLVQGQQLNPYLRLKVRRDHIIDDALVRLEMIAM
ENPADLKKQLYVEFEGEQGVDEGGVSKEFFQLVVE
EIFNPDIGMFTYDNNTKLFWFNPSSFETEGQFTLI
GIVLGLAIYNNTILDVHFPMVVYRKLMGKKGTFRD
LGDSHPVLYQSLKDLLEYEGSVEDDMMITFQISQT
DLFGNPMMYDLKENGDKIPITNENRKEFVNLYSDY
ILNKSVEKQFKAFRRGFHMVTNESPLKYLFRPEEI
ELLICGSRNLDFQALEETTEYDGGYTRESVVIREF
WEIVHSFTDEQKRLFLQNTTGTDRAPVGGLGKLKM
IIAKNGPDTERLPTSHTCFNVLLLPEYSSKEKLKE
RLLKAITYAKGFGML-890

### Modified Mouse Isoform #2

Nucleotide positions 253, 356, 373, 715, 716, 1120, 1121, 1825, 1827, 1828, 1829, 1924, 1925, 2467, and 2468
AA positions 85, 119, 125, 239, 374, 609, 610, 642, and 823
SEQ ID NO: 27, Mouse Ube3a isoform 2 with 8×N-glycan sites (bolded) and secretion signal (underlined). Bold and capitalized font provides examples of mutations to create a glycosylation site.

1-atgtacaggatgcaa ctcctgtctt gcattgcact
aagtcttgca cttgtcacaa acagt a tggccacagc
ttgtaaaaga tcaccaggagaatcccagtc
tgaggacatt gaagctagcc gaatgaagcg agcagctgca
aagcatctaatagaacgcta ctaccatcag ttaactgagg
gctgtggaaa tgaggcctgc acgaatgagt
tttgtgcttc ctgtccaact tttcttcgta
tggataacaa tAcagcagct attaaagccc
ttgagcttta taaaattaat gcaaaactct
gtgatcctca tccctccaag aaaggagcaa
gctcagctta ccttgagaac tcaaCaggtg
catctaacaa cAcagagata aaaatgaaca
agaaggaagg aaaagatttt aaagatgtga
tttacctaac tgaagagaaa gtatatgaaa
tttatgaatt ttgtagagag agtgaggatt
attccccttt aattcgtgta attggaagaa
tattttctag tgctgaggca ctggttctga
gctttcggaa agtcaaacag cacacaaagg
aggaattgaa atctcttcaa gaaaaggatg
aagacaagga tgaagatgaa aaggaaaaag
ctgcatgttc tgctgctgct atggaagaag
actcagaagc atcttcttca aggatgggtg
atagttcaca gggagacaac aatACacaaa
aattaggtcc tgatgatgtg actgtggata
ttgatgctat tagaagggtc tacagcagtt
tgctcgctaa tgaaaaatta gaaactgcct
tcctgaatgc acttgtatat ctgtcaccta
acgtggaatg tgatttgaca tatcataatg
tgtatactcg agatcctaat tatctcaatt
tgttcattat tgtaatggag aatagtaatc
tccacagtcc tgaatatctg gaaatggcgt
tgccattatt ttgcaaagct atgtgtaagc
taccccttga agctcaagga aaactgatta
ggctgtggtc taaatacagt gctgaccaga
ttcggagaat gatggaaaca tttcagcaac
ttattaccta caaagtcata agcaatgaat
ttaatagcACaaatctagtg aatgatgatg
atgccattgt tgctgcttca aagtgtttga
aaatggttta ctatgcaaat gtagtgggag
gggatgtgga cacaaatcat aatgaggaag
atgatgaaga acccatacct gagtccagcg
aattaacact tcaggagctt ctgggagatg
aaagaagaaa taagaaaggt cctcgagtgg
atccactaga aaccgaactt ggcgttaaaa
ctctagactg tcgaaaacca cttatctcct
ttgaagaatt cattaatgaa ccactgaatg
atgttctaga aatggacaaa gattatacct
ttttcaaagt tgaaacagag aacaaattct
cttttatgac atgtcccttt atattgaatg
ctgtcacaaa gaatctggga ttatattatg
acaatagaat tcgcatgtac agtgaaagaa
gaatcactgt tctttacagc ctagttcaag
gacagcagtt gaatccgtat ttgagactca
aagtcagacg tgaccatatt atagatgatg
cactggtccg gctagagatg attgctatgg
aaaatcctgc agacttgaag aagcagttgt
atgtggaatt tgaaggagaa caaggagtag
atgagggagg cgtttccaaa gagttttttc
agttggttgt ggaggaaatt tttaatccag
atattggtat gttcacatat gatAaTAAta
cgaaattatt ttggtttaat ccatcttctt
ttgaaactga gggtcagttt actctgattg
gcatagtcct gggtctggct atttacaata
atACtatact ggatgtccat tttcccatgg
ttgtatacag gaagctaatg gggaaaaaag
gaacctttcg tgacttggga gactctcacc
cagttttata tcagagttta aaggatttat
tggaatatga agggagtgtg gaagatgata
tgatgatcac tttccagata tcacagacag
atctttttgg taacccaatg atgtatgatc
taaaagaaaa tggtgataaa attccaatta
caaatgaaaa caggaaggaa tttgtcaatc
tctattcaga ctacattctc aataaatctg
tagaaaaaca attcaaggca tttcgcagag
gttttcatat ggtgactaat gaatcgccct
taaaatactt attcagacca gaagaaattg
aattgcttat atgtggaagc cggaatctag
atttccaggc actagaagaa actacagagt
atgacggtgg ctatacgagg gaatctgttg
tgattaggga gttctgggaa attgttcatt
cgtttacaga tgaacagaaa agactctttc
tgcagAAtac aacaggcaca gacagagcac
ctgttggagg actaggaaaa ttgaagatga
ttatagccaa aaatggccca gacacagaaa
ggttacctac atctcatact tgctttaatg
tccttttact tccggaatat tcaagcaaag
aaaaacttaa agagagattg ttgaaggcca
tcacatatgc caaaggattt ggcatgctgta
a-2673

SEQ ID NO: 28, Mouse Ube3a isoform 2 with 8×N-glycan sites (bolded) and secretion signal (underlined) aa sequence

MYRMQLLSCIALSLALVTNSMATACKRSPGESQSE
DIEASRMKRAAAKHLIERYYHQLTEGCGNEACTNE
FCASCPTFLRMDNNTAAIKALELYKINAKLCDPHP
SKKGASSAYLENSTGASNNTEIKMNKKEGKDFKDV
IYLTEEKVYEIYEFCRESEDYSPLIRVIGRIFSSA
EALVLSFRKVKQHTKEELKSLQEKDEDKDEDEKEK
AACSAAAMEEDSEASSSRMGDSSQGDNNTQKLGPD
DVTVDIDAIRRVYSSLLANEKLETAFLNALVYLSP
NVECDLTYHNVYTRDPNYLNLFIIVMENSNLHSPE
YLEMALPLFCKAMCKLPLEAQGKLIRLWSKYSADQ
IRRMMETFQQLITYKVISNEFNSTNLVNDDDAIVA
ASKCLKMVYYANVVGGDVDTNHNEEDDEEPIPESS
ELTLQELLGDERRNKKGPRVDPLETELGVKTLDCR
KPLISFEEFINEPLNDVLEMDKDYTFFKVETENKF
SFMTCPFILNAVTKNLGLYYDNRIRMYSERRITVL
YSLVQGQQLNPYLRLKVRRDHIIDDALVRLEMIAM
ENPADLKKQLYVEFEGEQGVDEGGVSKEFFQLVVE
EIFNPDIGMFTYDNNTKLFWFNPSSFETEGQFTLI
GIVLGLAIYNNTILDVHFPMVVYRKLMGKKGTFRD
LGDSHPVLYQSLKDLLEYEGSVEDDMMITFQISQT
DLFGNPMMYDLKENGDKIPITNENRKEFVNLYSDY
ILNKSVEKQFKAFRRGFHMVTNESPLKYLFRPEEI
ELLICGSRNLDFQALEETTEYDGGYTRESVVIREF
WEIVHSFTDEQKRLFLQNTTGTDRAPVGGLGKLKM
IIAKNGPDTERLPTSHTCFNVLLLPEYSSKEKLKE
RLLKAITYAKGFGML-890

### Modified Mouse Isoform #3

Nucleotide positions 190, 293, 310, 652, 653, 1057, 1058, 1762, 1764, 1765, 1766, 1861, 1862, 2404, and 2405
AA positions 64, 98, 104, 218, 353, 588, 589, 621, and 802
SEQ ID NO: 29, Mouse Ube3a isoform 3 with 8×N-glycan sites (bolded) and secretion signal (underlined). Bold and capitalized font provides examples of mutations to create a glycosylation site.

1-atgtacaggatgcaa ctcctgtctt gcattgcact
aagtcttgca cttgtcacaa acagt atgaa
gcgagcagct gcaaagcatc taatagaacg
ctactaccat cagttaactg agggctgtgg
aaatgaggcc tgcacgaatg agttttgtgc
ttcctgtcca acttttcttc gtatggataa
caatAcagca gctattaaag cccttgagct
ttataaaatt aatgcaaaac tctgtgatcc
tcatccctcc aagaaaggag caagctcagc
ttaccttgag aactcaaCag gtgcatctaa
caacAcagag ataaaaatga acaagaagga
aggaaaagat tttaaagatg tgatttacct
aactgaagag aaagtatatg aaatttatga
attttgtaga gagagtgagg attattcccc
tttaattcgt gtaattggaa gaatattttc
tagtgctgag gcactggttc tgagctttcg
gaaagtcaaa cagcacacaa aggaggaatt
gaaatctctt caagaaaagg atgaagacaa
ggatgaagat gaaaaggaaa aagctgcatg
ttctgctgct gctatggaag aagactcaga
agcatcttct tcaaggatgg gtgatagttc
acagggagac aacaatACac aaaaattagg
tcctgatgat gtgactgtgg atattgatgc
tattagaagg gtctacagca gtttgctcgc
taatgaaaaa ttagaaactg ccttcctgaa
tgcacttgta tatctgtcac ctaacgtgga
atgtgatttg acatatcata atgtgtatac
tcgagatcct aattatctca atttgttcat
tattgtaatg gagaatagta atctccacag
tcctgaatat ctggaaatgg cgttgccatt
attttgcaaa gctatgtgta agctacccct
tgaagctcaa ggaaaactga ttaggctgtg
gtctaaatac agtgctgacc agattcggag
aatgatggaa acatttcagc aacttattac
ctacaaagtc ataagcaatg aatttaatag
cACaaatcta gtgaatgatg atgatgccat
tgttgctgct tcaaagtgtt tgaaaatggt
ttactatgca aatgtagtgg gaggggatgt
ggacacaaat cataatgagg aagatgatga
agaacccata cctgagtcca gcgaattaac
acttcaggag cttctgggag atgaaagaag
aaataagaaa ggtcctcgag tggatccact
agaaaccgaa cttggcgtta aaactctaga
ctgtcgaaaa ccacttatct cctttgaaga
attcattaat gaaccactga atgatgttct
agaaatggac aaagattata cctttttcaa
agttgaaaca gagaacaaat tctcttttat
gacatgtccc tttatattga atgctgtcac
aaagaatctg ggattatatt atgacaatag
aattcgcatg tacagtgaaa gaagaatcac
tgttctttac agcctagttc aaggacagca
gttgaatccg tatttgagac tcaaagtcag
acgtgaccat attatagatg atgcactggt
ccggctagag atgattgcta tggaaaatcc
tgcagacttg aagaagcagt tgtatgtgga
atttgaagga gaacaaggag tagatgaggg
aggcgtttcc aaagagtttt ttcagttggt
tgtggaggaa atttttaatc cagatattgg
tatgttcaca tatgatAaTAAtacgaaatt
attttggttt aatccatctt cttttgaaac
tgagggtcag tttactctga ttggcatagt
cctgggtctg gctatttaca ataatACtat
actggatgtc cattttccca tggttgtata
caggaagcta atggggaaaa aaggaacctt
tcgtgacttg ggagactctc acccagtttt
atatcagagt ttaaaggatt tattggaata
tgaagggagt gtggaagatg atatgatgat
cactttccag atatcacaga cagatctttt
tggtaaccca atgatgtatg atctaaaaga
aaatggtgat aaaattccaa ttacaaatga
aaacaggaag gaatttgtca atctctattc
agactacatt ctcaataaat ctgtagaaaa
acaattcaag gcatttcgca gaggttttca
tatggtgact aatgaatcgc ccttaaaata
cttattcaga ccagaagaaa ttgaattgct
tatatgtgga agccggaatc tagatttcca
ggcactagaa gaaactacag agtatgacgg
tggctatacg agggaatctg ttgtgattag
ggagttctgg gaaattgttc attcgtttac
agatgaacag aaaagactct ttctgcagAA
tacaacaggc acagacagag cacctgttgg
aggactagga aaattgaaga tgattatagc
caaaaatggc ccagacacag aaaggttacc
tacatctcat acttgcttta atgtcctttt
acttccggaa tattcaagca aagaaaaact
taaagagaga ttgttgaagg ccatcacata
tgccaaagga tttggcatgc tgtaa-2610

SEQ ID NO: 30, Mouse Ube3a isoform 3 with 8×N-glycan sites (bolded) and secretion signal (underlined) aa sequence

1-MYRMQLLSCIALSLALVTNSMKRAAAKHLIERY
YHQLTEGCGNEACTNEFCASCPTFLRMDNNTAAIK
ALELYKINAKLCDPHPSKKGASSAYLENSTGASNN
TEIKMNKKEGKDFKDVIYLTEEKVYEIYEFCRESE
DYSPLIRVIGRIFSSAEALVLSFRKVKQHTKEELK
SLQEKDEDKDEDEKEKAACSAAAMEEDSEASSSRM
GDSSQGDNNTQKLGPDDVTVDIDAIRRVYSSLLAN
EKLETAFLNALVYLSPNVECDLTYHNVYTRDPNYL
NLFIIVMENSNLHSPEYLEMALPLFCKAMCKLPLE
AQGKLIRLWSKYSADQIRRMMETFQQLITYKVISN
EFNSTNLVNDDDAIVAASKCLKMVYYANVVGGDVD
TNHNEEDDEEPIPESSELTLQELLGDERRNKKGPR
VDPLETELGVKTLDCRKPLISFEEFINEPLNDVLE
MDKDYTFFKVETENKFSFMTCPFILNAVTKNLGLY
YDNRIRMYSERRITVLYSLVQGQQLNPYLRLKVRR
DHIIDDALVRLEMIAMENPADLKKQLYVEFEGEQG
VDEGGVSKEFFQLVVEEIFNPDIGMFTYDNNTKLF
WFNPSSFETEGQFTLIGIVLGLAIYNNTILDVHFP
MVVYRKLMGKKGTFRDLGDSHPVLYQSLKDLLEYE
GSVEDDMMITFQISQTDLFGNPMMYDLKENGDKIP
ITNENRKEFVNLYSDYILNKSVEKQFKAFRRGFHM
VTNESPLKYLFRPEEIELLICGSRNLDFQALEETT
EYDGGYTRESVVIREFWEIVHSFTDEQKRLFLQNT
TGTDRAPVGGLGKLKMIIAKNGPDTERLPTSHTCF
NVLLLPEYSSKEKLKERLLKAITYAKGFGML-869

SEQ ID NO: 31, a nucleotide sequence shown in FIG. 16A, which is a fragment of Homo sapiens ubiquitin protein ligase E3A (UBE3A), transcript variant 5, having an NCBI Reference Number NM_001354506, and comprising a wildtype Ube3a CDS (i.e., SEQ ID NO: 7) starting from the capitalized ATG and ending at the capitalized TAA. Other capitalized nucleotide residues provide potential mutation sites to create glycosylation sites

ctg gtg gag acc aga tca gga gaa cct
cag tct gac gac att gaa gct agc cga
ATG aag cga gca gct gca aag cat cta
ata gaa cgc tac tac cac cag tta act
gag ggc tgt gga aat gaa gcc tgc acg
aat gag ttt tgt gct tcc tgt cca act
ttt ctt cgt atg gat aat aat Gca gca
gct att aaa gcc ctc gag ctt tat aag
att aat gca aaa ctc tgt gat cct cat
ccc tcc aag aaa gga gca agc tca gct
tac ctt gag aac tcg aAa ggt gcc ccc
aac aac Tcc tgc tct gag ata aaa atg
aac aag aaa ggc gct aga att gat ttt
aaa gat gtg act tac tta aca gaa gag
aag gta tat gaa att ctt gaa tta tgt
aga gaa aga gag gat tat tcc cct tta
atc cgt gtt att gga aga gtt ttt tct
agt gct gag gca ttg gta cag agc ttc
cgg aaa gtt aaa caa cac acc aag gaa
gaa ctg aaa tct ctt caa gca aaa gat
gaa gac aaa gat gaa gat gaa aag gaa
aaa gct gca tgt tct gct gct gct atg
gaa gaa gac tca gaa gca tct tcc tca
agg ata ggt gat agc tca cag gga gac
aac aat TTg caa aaa tta ggc cct gat
gat gtg tct gtg gat att gat gcc att
aga agg gtc tac acc aga ttg ctc tct
aat gaa aaa att gaa act gcc ttt ctc
aat gca ctt gta tat ttg tca cct aac
gtg gaa tgt gac ttg acg tat cac aat
gta tac tct cga gat cct aat tat ctg
aat ttg ttc att atc gta atg gag aat
aga aat ctc cac agt cct gaa tat ctg
gaa atg gct ttg cca tta ttt tgc aaa
gcg atg agc aag cta ccc ctt gca gcc
caa gga aaa ctg atc aga ctg tgg tct
aaa tac aat gca gac cag att cgg aga
atg atg gag aca ttt cag caa ctt att
act tat aaa gtc ata agc aat gaa ttt
aac agt CGa aat cta gtg aat gat gat
gat gcc att gtt gct gct tcg aag tgc
ttg aaa atg gtt tac tat gca aat gta
gtg gga ggg gaa gtg gac aca aat cac
aat gaa gaa gat gat gaa gag ccc atc
cct gag tcc agc gag ctg aca ctt cag
gaa ctt ttg gga gaa gaa aga aga aac
aag aaa ggt cct cga gtg gac ccc ctg
gaa act gaa ctt ggt gtt aaa acc ctg
gat tgt cga aaa cca ctt atc cct ttt
gaa gag ttt att aat gaa cca ctg aat
gag gtt cta gaa atg gat aaa gat tat
act ttt ttc aaa gta gaa aca gag aac
aaa ttc tct ttt atg aca tgt ccc ttt
ata ttg aat gct gtc aca aag aat ttg
gga tta tat tat gac aat aga att cgc
atg tac agt gaa cga aga atc act gtt
ctc tac agc tta gtt caa gga cag cag
ttg aat cca tat ttg aga ctc aaa gtt
aga cgt gac cat atc ata gat gat gca
ctt gtc cgg cta gag atg atc gct atg
gaa aat cct gca gac ttg aag aag cag
ttg tat gtg gaa ttt gaa gga gaa caa
gga gtt gat gag gga ggt gtt tcc aaa
gaa ttt ttt cag ctg gtt gtg gag gaa
atc ttc aat cca gat att ggt atg ttc
aca tac gat GaA tct aca aaa ttg ttt
tgg ttt aat cca tct tct ttt gaa act
gag ggt cag ttt act ctg att ggc ata
gta ctg ggt ctg gct att tac aat aac
TGt ata ctg gat gta cat ttt ccc atg
gtt gtc tac agg aag cta atg ggg aaa
aaa gga act ttt cgt gac ttg gga gac
tct cac cca gtt cta tat cag agt tta
aaa gat tta ttg gag tat gaa ggg aat
gtg gaa gat gac atg atg atc act ttc
cag ata tca cag aca gat ctt ttt ggt
aac cca atg atg tat gat cta aag gaa
aat ggt gat aaa att cca att aca aat
gaa aac agg aag gaa ttt gtc aat ctt
tat tct gac tac att ctc aat aaa tca
gta gaa aaa cag ttc aag gct ttt cgg
aga ggt ttt cat atg gtg acc aat gaa
tct ccc tta aag tac tta ttc aga cca
gaa gaa att gaa ttg ctt ata tgt gga
agc cgg aat cta gat ttc caa gca cta
gaa gaa act aca gaa tat gac ggt ggc
tat acc agg gac tct gtt ctg att agg
gag ttc tgg gaa atc gtt cat tca ttt
aca gat gaa cag aaa aga ctc ttc ttg
cag TTt aCA acg ggc aca gac aga gca
cct gtg gga gga cta gga aaa tta aag
atg att ata gcc aaa aat ggc cca gac
aca gaa agg tta cct aca tct cat act
tgc ttt aat gtg ctt tta ctt ccg gaa
tac tca agc aaa gaa aaa ctt aaa gag
aga ttg ttg aag gcc atc acg tat gcc
aaa gga ttt ggc atg ctg TAA aac aaa
aca aaa caa aat aaa aca aaa

SEQ ID NO: 32, an amino acid sequence shown in FIG. 16A comprising a wild type Ube3a protein of SEQ ID NO: 8. Non-capitalized font indicates NetNGlyC predicted N-glycosylation sites (marked in FIG. 16A with 82,579,700,719 provided as the residue number of the beginning of the glycosylation site in SEQ ID NO: 8). Bold font provides some potential glycosylation site that can be created by mutation as identified herein. Italic font indicates potential glycosylation site that can be created by mutations the two amino acids preceding an S or a T. Underlines denotes potential glycosylation sites that can be created by mutation the two amino acids succeeding an N.

LVETRSGEPQSDDIEASRMKRAAAKHLIERYYHQLT
EGCGNEACTNEFCASCPTFLRMDNNAAAIKALELY
KINAKLCDPHPSKKGASSAYLENSKGAPnnsCSEI
KMNKKGARIDFKDVTYLTEEKVYEILELCREREDY
SPLIRVIGRVFSSAEALVQSFRKVKQHTKEELKSL
QAKDEDKDEDEKEKAACSAAAMEEDSEASSSRIGD
SSQGDNNLQKLGPDDVSVDIDAIRRVYTRLLSNEK
IETAFLNALVYLSPNVECDLTYHNVYSRDPNYLNL
FIIVMENRNLHSPEYLEMALPLFCKAMSKLPLAAQ
GKLIRLWSKYNADQIRRMMETFQQLITYKVISNEF
NSRNLVNDDDAIVAASKCLKMVYYANVVGGEVDTN
HNEEDDEEPIPESSELTLQELLGEERRNKKGPRVD
PLETELGVKTLDCRKPLIPFEEFINEPLNEVLEMD
KDYTFFKVETENKFSFMTCPFILNAVTKNLGLYYD
NRIRMYSERRITVLYSLVQGQQLNPYLRLKVRRDH
IIDDALVRLEMIAMENPADLKKQLYVEFEGEQGVD
EGGVSKEFFQLVVEEIFNPDIGMFTYDESTKLFWF
npsSFETEGQFTLIGIVLGLAIYNNCILDVHFPMV
VYRKLMGKKGTFRDLGDSHPVLYQSLKDLLEYEGN
VEDDMMITFQISQTDLFGNPMMYDLKENGDKIPIT
NENRKEFVNLYSDYILnksVEKQFKAFRRGFHMVT
nesPLKYLFRPEEIELLICGSRNLDFQALEETTEY
DGGYTRDSVLIREFWEIVHSFTDEQKRLFLQFTTG
TDRAPVGGLGKLKMIIAKNGPDTERLPTSHTCFNV
LLLPEYSSKEKLKERLLKAITYAKGFGML

SEQ ID NO: 33, an amino acid sequence show in FIG. 16A.

### NKTKQNKTK

CMV promoter sequence 3.
SEQ ID NO: 34
Cgttacataacttacggtaaatggcccgcctggctg
accgcccaacgacccccgcccattgacgtcaataa
tgacgtatgttcccatagtaacgccaatagggact
ttccattgacgtcaatgggtggagtatttacggta
aactgcccacttggcagtacatcaagtgtatcata
tgccaagtacgccccctattgacgtcaatgacggt
aaatggcccgcctggcattatgcccagtacatgac
cttatgggactttcctacttggcagtacatctacg
tattagtcatcgctattaccatggtgatgcggttt
gtggcagtacatcaatgggcgtggatagcggtttg
actcacggggatttccaagtctccaccccattgac
gtcaatgggagtttgttttggcaccaaaatcaacg
ggactttccaaaatgtcgtaacaactccgccccat
tgacgcaaatgggcggtaggcgtgtacggtgggag
gtctatataagcagagctcgtttagtgaaccg,

SEQ ID NO: 35, CCLc-MNDU3-X vector, wherein a CMV promoter of SEQ ID NO: 34 is shown in bold, italic and capitalized font and a MNDU3 promoter of SEQ ID NO: 3 is shown in bold, italic and non-capitalized font.

1-caggtggcacttttcggggaaatgtgcgcggaa
cccctatttgtttatttttctaaatacattcaaat
atgtatccgctcatgagacaataaccctgataaat
gcttcaataatattgaaaaaggaagagtatgagta
ttcaacatttccgtgtcgcccttattccctttttt
gcggcattttgccttcctgtttttgctcacccaga
aacgctggtgaaagtaaaagatgctgaagatcagt
tgggtgcacgagtgggttacatcgaactggatctc
aacagcggtaagatccttgagagttttcgccccga
agaacgttttccaatgatgagcacttttaaagttc
tgctatgtggcgcggtattatcccgtattgacgcc
gggcaagagcaactcggtcgccgcatacactattc
tcagaatgacttggttgagtactcaccagtcacag
aaaagcatcttacggatggcatgacagtaagagaa
ttatgcagtgctgccataaccatgagtgataacac
tgcggccaacttacttctgacaacgatcggaggac
cgaaggagctaaccgcttttttgcacaacatgggg
gatcatgtaactcgccttgatcgttgggaaccgga
gctgaatgaagccataccaaacgacgagcgtgaca
ccacgatgcctgtagcaatggcaacaacgttgcgc
aaactattaactggcgaactacttactctagcttc
ccggcaacaattaatagactggatggaggcggata
aagttgcaggaccacttctgcgctcggcccttccg
gctggctggtttattgctgataaatctggagccgg
tgagcgtgggtctcgcggtatcattgcagcactgg
ggccagatggtaagccctcccgtatcgtagttatc
tacacgacggggagtcaggcaactatggatgaacg
aaatagacagatcgctgagataggtgcctcactga
ttaagcattggtaactgtcagaccaagtttactca
tatatactttagattgatttaaaacttcattttta
atttaaaaggatctaggtgaagatcctttttgata
atctcatgaccaaaatcccttaacgtgagttttcg
ttccactgagcgtcagaccccgtagaaaagatcaa
aggatcttcttgagatcctttttttctgcgcgtaa
tctgctgcttgcaaacaaaaaaaccaccgctacca
gcggtggtttgtttgccggatcaagagctaccaac
tctttttccgaaggtaactggcttcagcagagcgc
agataccaaatactgtccttctagtgtagccgtag
ttaggccaccacttcaagaactctgtagcaccgcc
tacatacctcgctctgctaatcctgttaccagtgg
ctgctgccagtggcgataagtcgtgtcttaccggg
ttggactcaagacgatagttaccggataaggcgca
gcggtcgggctgaacggggggttcgtgcacacagc
ccagcttggagcgaacgacctacaccgaactgaga
tacctacagcgtgagctatgagaaagcgccacgct
tcccgaagggagaaaggcggacaggtatccggtaa
gcggcagggtcggaacaggagagcgcacgagggag
cttccagggggaaacgcctggtatctttatagtcc
tgtcgggtttcgccacctctgacttgagcgtcgat
ttttgtgatgctcgtcaggggggcggagcctatgg
aaaaacgccagcaacgcggcctttttacggttcct
ggccttttgctggccttttgctcacatgttctttc
ctgcgttatcccctgattctgtggataaccgtatt
accgcctttgagtgagctgataccgctcgccgcag
ccgaacgaccgagcgcagcgagtcagtgagcgagg
aagcggaagagcgcccaatacgcaaaccgcctctc
cccgcgcgttggccgattcattaatgcagctggca
cgacaggtttcccgactggaaagcgggcagtgagc
gcaacgcaattaatgtgagttagctcactcattag
gcaccccaggctttacactttatgcttccggctcg
tatgttgtgtggaattgtgagcggataacaatttc
acacaggaaacagctatgaccatgattacgccaag
cgcgcaattaaccctcactaaagggaacaaaagct
ggagctgcaagcttggccattgcatacgttgtatc
catatcataatatgtacatttatattggctcatgt
ccaacattaccgccatgttgacattgattattgac
tagttattaatagtaatcaattacggggtcattag
ttcatagcccatatatggagttccg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gggtctctctggttagaccagatctgagcctgggag
ctctctggctaactagggaacccactgcttaagcc
tcaataaagcttgccttgagtgcttcaagtagtgt
gtgcccgtctgttgtgtgactctggtaactagaga
tccctcagacccttttagtcagtgtggaaaatctc
tagcagtggcgcccgaacagggacctgaaagcgaa
agggaaaccagaggagctctctcgacgcaggactc
ggcttgctgaagcgcgcacggcaagaggcgagggg
cggcgactggtgagtacgccaaaaattttgactag
cggaggctagaaggagagagatgggtgcgagagcg
tcagtattaagcgggggagaattagatcgcgatgg
gaaaaaattcggttaaggccagggggaaagaaaaa
atataaattaaaacatatagtatgggcaagcaggg
agctagaacgattcgcagttaatcctggcctgtta
gaaacatcagaaggctgtagacaaatactgggaca
gctacaaccatcccttcagacaggatcagaagaac
ttagatcattatataatacagtagcaaccctctat
tgtgtgcatcaaaggatagagataaaagacaccaa
ggaagctttagacaagatagaggaagagcaaaaca
aaagtaagaccaccgcacagcaagcggccgctgat
cttcagacctggaggaggagatatgagggacaatt
ggagaagtgaattatataaatataaagtagtaaaa
attgaaccattaggagtagcacccaccaaggcaaa
gagaagagtggtgcagagagaaaaaagagcagtgg
gaataggagctttgttccttgggttcttgggagca
gcaggaagcactatgggcgcagcgtcaatgacgct
gacggtacaggccagacaattattgtctggtatag
tgcagcagcagaacaatttgctgagggctattgag
gcgcaacagcatctgttgcaactcacagtctgggg
catcaagcagctccaggcaagaatcctggctgtgg
aaagatacctaaaggatcaacagctcctggggatt
tggggttgctctggaaaactcatttgcaccactgc
tgtgccttggaatgctagttggagtaataaatctc
tggaacagattggaatcacacgacctggatggagt
gggacagagaaattaacaattacacaagcttaata
cactccttaattgaagaatcgcaaaaccagcaaga
aaagaatgaacaagaattattggaattagataaat
gggcaagtttgtggaattggtttaacataacaaat
tggctgtggtatataaaattattcataatgatagt
aggaggcttggtaggtttaagaatagtttttgctg
tactttctatagtgaatagagttaggcagggatat
tcaccattatcgtttcagacccacctcccaacccc
gaggggacccgacaggcccgaaggaatagaagaag
aaggtggagagagagacagagacagatccattcga
ttagtgaacggatctcgacggtatcgataagctaa
ttcacaaatggcagtattcatccacaattttaaaa
gaaaaggggggattggggggtacagtgcaggggaa
agaatagtagacataatagcaacagacatacaaac
taaagaattacaaaaacaaattacaaaaattcaaa
attttcgggtttattacagggacagcagagatcca
gtttgggaattagcttgat

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GTTAACAGATCCCCCGGGt
cgactgatcaaattcgagctcggtacctttaagac
caatgacttacaaggcagctgtagatcttagccac
tttttaaaagaaaaggggggactggaagggctaat
tcactcccaacgaagacaagatctgctttttgctt
gtactgggtctctctggttagaccagatctgagcc
tgggagctctctggctaactagggaacccactgct
taagcctcaataaagcttgccttgagtgcttcaag
tagtgtgtgcccgtctgttgtgtgactctggtaac
tagagatccctcagacccttttagtcagtgtggaa
aatctctagcagtagtagttcatgtcatcttatta
ttcagtatttataacttgcaaagaaatgaatatca
gagagtgagaggaacttgtttattgcagcttataa
tggttacaaataaagcaatagcatcacaaatttca
caaataaagcatttttttcactgcattctagttgt
ggtttgtccaaactcatcaatgtatcttatcatgt
ctggctctagctatcccgcccctaactccgcccat
cccgcccctaactccgcccagttccgcccattctc
cgccccatggctgactaattttttttatttatgca
gaggccgaggccgcctcggcctctgagctattcca
gaagtagtgaggaggcttttttggaggcctaggct
tttgcgtcgagacgtacccaattcgccctatagtg
agtcgtattacgcgcgctcactggccgtcgtttta
caacgtcgtgactgggaaaaccctggcgttaccca
acttaatcgccttgcagcacatccccctttcgcca
gctggcgtaatagcgaagaggcccgcaccgatcgc
ccttcccaacagttgcgcagcctgaatggcgaatg
gcgcgacgcgccctgtagcggcgcattaagcgcgg
cgggtgtggtggttacgcgcagcgtgaccgctaca
cttgccagcgccctagcgcccgctcctttcgcttt
cttcccttcctttctcgccacgttcgccggctttc
cccgtcaagctctaaatcgggggctccctttaggg
ttccgatttagtgctttacggcacctcgaccccaa
aaaacttgattagggtgatggttcacgtagtgggc
catcgccctgatagacggtttttcgccctttgacg
ttggagtccacgttctttaatagtggactcttgtt
ccaaactggaacaacactcaaccctatctcggtct
attcttttgatttataagggattttgccgatttcg
gcctattggttaaaaaatgagctgatttaacaaaa
atttaacgcgaattttaacaaaatattaacgttta
caatttcc-6612

1. A polynucleotide encoding the Ube3a polypeptide the biological equivalent thereof of claim 35.

2.-16. (canceled)

17. The polynucleotide of claim 1, comprising a nucleotide (nt) sequence selected from any one of the following: nt61 to nt2619 of SEQ ID NO: 13, nt61 to nt2688 of SEQ ID NO: 15, nt61 to nt2679 of SEQ ID NO: 17, nt61 to nt2673 of SEQ ID NO: 25, nt61 to nt2673 of SEQ ID NO: 27, nt61 to nt2610 of SEQ ID NO: 29; SEQ ID NO: 13, SEQ ID NO: 15, SEQ ID NO: 17, SEQ ID NO: 25, SEQ ID NO: 27, SEQ ID NO: 29, or a sequence having at least about 90%, or at least about 95%, or at least about 99% identity to each thereof.

18. The polynucleotide of claim 1, wherein the Ube3a polypeptide or the biological equivalent thereof is derived from a wildtype human Ube3a protein or a wildtype murine Ube3a protein.

19. The polynucleotide of claim 18, wherein the wildtype human Ube3a protein comprises an amino acid sequence of any one of SEQ ID NOs: 8, 10, or 12, and wherein the wildtype murine Ube3a protein comprises an amino acid sequence of any one of SEQ ID NOs: 20, 22 or 24.

20.-25. (canceled)

26. A polynucleotide that is reverse or complement or reverse-complement to the polynucleotide of claim 1.

27. A vector comprising the polynucleotide of claim 1.

28.-33. (canceled)

34. The vector of claim 27, further comprising a regulatory sequence operatively linked to the polynucleotide and directing the replication of the polynucleotide.

35. A recombinant Ubiquitin Protein Ligase E3A (Ube3a) polypeptide or a biological equivalent thereof whose amino acid sequence differs from a wildtype Ube3a protein at least by addition of one or more glycosylation sites.

36. The recombinant Ube3a polypeptide or the biological equivalent thereof of claim 35, wherein the glycosylation is an N-linked glycosylation and wherein the glycosylation site comprise a consensus sequence of NXaaT or NXaaS, wherein Xaa is any amino acid residue optionally except proline (P).

37. The recombinant Ube3a polypeptide or the biological equivalent thereof of claim 35, wherein the glycosylation sites are at amino acid (aa) positions of the polypeptide or protein corresponding to one or more of those selected from the following:
aa62 to aa64 of SEQ ID NO: 14, aa96 to aa98 of SEQ ID NO: 14, aa102 to aa104 of
SEQ ID NO: 14, aa219 to aa221 of SEQ ID NO: 14, aa354 to aa356 of SEQ ID NO: 14, aa591 to aa593 of SEQ ID NO: 14, aa622 to aa624 of SEQ ID NO: 14, aa805 to aa807 of SEQ ID NO: 14;
aa85 to aa87 of SEQ ID NO: 16, aa119 to aa121 of SEQ ID NO: 16, aa125 to aa127 of SEQ ID NO: 16, aa242 to aa244 of SEQ ID NO: 16, aa377 to aa379 of SEQ ID NO: 16, aa614 to aa616 of SEQ ID NO: 16, aa645 to aa647 of SEQ ID NO: 16, aa828 to aa830 of SEQ ID NO: 16;
aa82 to aa84 of SEQ ID NO: 18, aa116 to aa118 of SEQ ID NO: 18, aa 122 to aa124 of SEQ ID NO: 18, aa239 to aa241 of SEQ ID NO: 18, aa374 to aa376 of SEQ ID NO: 18, aa611 to aa613 of SEQ ID NO: 18, aa642 to aa644 of SEQ ID NO: 18, aa825 to aa827 of SEQ ID NO: 18;
aa83 to aa85 of SEQ ID NO: 26 or 28, aa117 to aa119 of SEQ ID NO: 26 or 28, aa123 to aa125 of SEQ ID NO: 26 or 28, aa 237 to aa239 of SEQ ID NO: 26 or 28, aa372 to aa374 of SEQ ID NO: 26 or 28, aa609 to aa611 of SEQ ID NO: 26 or 28, aa640 to aa642 of SEQ ID NO: 26 or 28, aa823 to aa825 of SEQ ID NO: 26 or 28;
aa62 to aa64 of SEQ ID NO: 30, aa96 to aa98 of SEQ ID NO: 30, aa102 to aa104 of SEQ ID NO: 30, aa216 to aa218 of SEQ ID NO: 30, aa351 to aa353 of SEQ ID NO: 30, aa588 to aa590 of SEQ ID NO: 30, aa619 to aa621 of SEQ ID NO: 30, or aa802 to aa804 of SEQ ID NO: 30.

38. The recombinant Ube3a polypeptide or the biological equivalent thereof of claim 35, wherein the Ube3a polypeptide or a biological equivalent thereof comprises eight glycosylation sites at an amino acid (aa) positions corresponding to the eight aa positions as identified in any one of the following (a) to (e):
(a) aa62 to aa64 of SEQ ID NO: 14, aa96 to aa98 of SEQ ID NO: 14, aa102 to aa104 of SEQ ID NO: 14, aa219 to aa221 of SEQ ID NO: 14, aa354 to aa356 of SEQ ID NO: 14, aa591 to aa 593 of SEQ ID NO: 14, aa622 to aa624 of SEQ ID NO: 14, aa 805 to aa 807 of SEQ ID NO: 14;
(b) aa85 to aa87 of SEQ ID NO: 16, aa119 to aa121 of SEQ ID NO: 16, aa125 to aa127 of SEQ ID NO: 16, aa242 to aa244 of SEQ ID NO: 16, aa377 to aa379 of SEQ ID NO: 16, aa614 to aa616 of SEQ ID NO: 16, aa645 to aa647 of SEQ ID NO: 16, aa828 to aa830 of SEQ ID NO: 16;
(c) aa82 to aa84 of SEQ ID NO: 18, aa116 to aa118 of SEQ ID NO: 18, aa 122 to aa124 of SEQ ID NO: 18, aa239 to aa241 of SEQ ID NO: 18, aa374 to aa376 of SEQ ID NO: 18, aa611 to aa613 of SEQ ID NO: 18, aa642 to aa644 of SEQ ID NO: 18, aa825 to aa827 of SEQ ID NO: 18;
(d) aa83 to aa85 of SEQ ID NO: 26 or 28, aa117 to aa119 of SEQ ID NO: 26 or 28, aa123 to aa125 of SEQ ID NO: 26 or 28, aa 237 to aa239 of SEQ ID NO: 26 or 28, aa372 to aa374 of SEQ ID NO: 26 or 28, aa609 to aa611 of SEQ ID NO: 26 or 28, aa640 to aa642 of SEQ ID NO: 26 or 28, aa823 to aa825 of SEQ ID NO: 26 or 28;
(e) aa62 to aa64 of SEQ ID NO: 30, aa96 to aa98 of SEQ ID NO: 30, aa102 to aa104 of SEQ ID NO: 30, aa216 to aa218 of SEQ ID NO: 30, aa351 to aa353 of SEQ ID NO: 30, aa588 to aa590 of SEQ ID NO: 30, aa619 to aa621 of SEQ ID NO: 30, or aa802 to aa804 of SEQ ID NO: 30.

39. The recombinant Ube3a polypeptide or the biological equivalent thereof of claim 35, comprising one or more of mutated amino acid residue(s) at aa position(s) corresponding to one or more of those selected from the following, thereby forming one or more of glycosylation site(s):
aa64 of SEQ ID NO: 14, aa98 of SEQ ID NO: 14, aa104 of SEQ ID NO: 14, aa221 of SEQ ID NO: 14, aa356 of SEQ ID NO: 14, aa591 of SEQ ID NO: 14, aa624 of SEQ ID NO: 14, aa805 of SEQ ID NO: 14, aa 806 of SEQ ID NO: 14;
aa87 of SEQ ID NO: 16, aa121 of SEQ ID NO: 16, aa127 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, aa829 of SEQ ID NO: 16;
aa84 of SEQ ID NO: 18, aa118 of SEQ ID NO: 18, aa124 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, aa826 of SEQ ID NO: 18;
aa85 of SEQ ID NO: 26 or 28, aa119 of SEQ ID NO: 26 or 28, aa125 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, aa823 of SEQ ID NO: 26 or 28;
aa64 of SEQ ID NO: 30, aa98 of SEQ ID NO: 30, aa104 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, or aa802 of SEQ ID NO: 30,
optionally wherein any one or any two or all three amino acid residue(s) in at least one of the glycosylation sites is or are mutated compared to a wild type Ube3a polypeptide or protein, thereby constituting the glycosylation site.

40. The recombinant Ube3a polypeptide or the biological equivalent thereof of claim 35, comprising nine mutated amino acid residues at aa positions corresponding to those identified in any one of the following (a) to (e), thereby forming eight glycosylation sites:
(a) aa64 of SEQ ID NO: 14, aa98 of SEQ ID NO: 14, aa104 of SEQ ID NO: 14, aa221 of SEQ ID NO: 14, aa356 of SEQ ID NO: 14, aa591 of SEQ ID NO: 14, aa624 of SEQ ID NO: 14, aa805 of SEQ ID NO: 14, and aa806 of SEQ ID NO: 14;
(b) aa87 of SEQ ID NO: 16, aa121 of SEQ ID NO: 16, aa127 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, and aa829 of SEQ ID NO: 16;
(c) aa84 of SEQ ID NO: 18, aa118 of SEQ ID NO: 18, aa124 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, and aa826 of SEQ ID NO: 18;
(d) aa85 of SEQ ID NO: 26 or 28, aa119 of SEQ ID NO: 26 or 28, aa125 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, and aa823 of SEQ ID NO: 26 or 28; or
(e) aa64 of SEQ ID NO: 30, aa98 of SEQ ID NO: 30, aa104 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, and aa802 of SEQ ID NO: 30.

41. The recombinant Ube3a polypeptide or the biological equivalent thereof of claim 35, comprising eight mutated amino acid residues at aa positions corresponding to those identified in any one of the following (a) to (e), thereby forming seven glycosylation sites:
(a) aa64 of SEQ ID NO: 14, aa98 of SEQ ID NO: 14, aa221 of SEQ ID NO: 14, aa356 of SEQ ID NO: 14, aa591 of SEQ ID NO: 14, aa624 of SEQ ID NO: 14, aa805 of SEQ ID NO: 14, and aa806 of SEQ ID NO: 14;
(b) aa87 of SEQ ID NO: 16, aa121 of SEQ ID NO: 16, aa244 of SEQ ID NO: 16, aa379 of SEQ ID NO: 16, aa614 of SEQ ID NO: 16, aa647 of SEQ ID NO: 16, aa828 of SEQ ID NO: 16, and aa829 of SEQ ID NO: 16;
(c) aa84 of SEQ ID NO: 18, aa118 of SEQ ID NO: 18, aa241 of SEQ ID NO: 18, aa376 of SEQ ID NO: 18, aa611 of SEQ ID NO: 18, aa644 of SEQ ID NO: 18, aa825 of SEQ ID NO: 18, and aa826 of SEQ ID NO: 18;
(d) aa85 of SEQ ID NO: 26 or 28, aa119 of SEQ ID NO: 26 or 28, aa239 of SEQ ID NO: 26 or 28, aa374 of SEQ ID NO: 26 or 28, aa609 of SEQ ID NO: 26 or 28, aa610 of SEQ ID NO: 26 or 28, aa642 of SEQ ID NO: 26 or 28, and aa823 of SEQ ID NO: 26 or 28; or
(e) aa64 of SEQ ID NO: 30, aa98 of SEQ ID NO: 30, aa218 of SEQ ID NO: 30, aa353 of SEQ ID NO: 30, aa588 of SEQ ID NO: 30, aa589 of SEQ ID NO: 30, aa621 of SEQ ID NO: 30, and aa802 of SEQ ID NO: 30.

42. The recombinant Ube3a polypeptide or the biological equivalent thereof of claim 39, wherein the formed glycosylation site comprises a consensus sequence of NXaaT or NXaaS, wherein Xaa is any amino acid residue optionally except proline (P).

43. The recombinant Ube3a polypeptide or the biological equivalent thereof of claim 39, wherein the mutated amino acid residue(s) is/are selected from one or more of:
T or S at an aa position corresponding to aa64 of SEQ ID NO: 14, T or S at an aa position corresponding to aa98 of SEQ ID NO: 14, T or S at an aa position corresponding to aa104 of SEQ ID NO: 14, T or S at an aa position corresponding to aa221 of SEQ ID NO: 14, T or S at an aa position corresponding to aa356 of SEQ ID NO: 14, N at an aa position corresponding to aa591 of SEQ ID NO: 14, T or S at an aa position corresponding to aa624 of SEQ ID NO: 14, N at an aa position corresponding to aa805 of SEQ ID NO: 14, and N at an aa position corresponding to aa806 of SEQ ID NO: 14;
T or S at an aa position corresponding to aa87 of SEQ ID NO: 16, T or S at an aa position corresponding to aa121 of SEQ ID NO: 16, T or S at an aa position corresponding to aa127 of SEQ ID NO: 16, T or S at an aa position corresponding to aa244 of SEQ ID NO: 16, T or S at an aa position corresponding to aa379 of SEQ ID NO: 16, N at an aa position corresponding to aa614 of SEQ ID NO: 16, T or S at an aa position corresponding to aa647 of SEQ ID NO: 16, N at an aa position corresponding to aa828 of SEQ ID NO: 16, and N at an aa position corresponding to aa829 of SEQ ID NO: 16;
T or S at an aa position corresponding to aa84 of SEQ ID NO: 18, T or S at an aa position corresponding to aa118 of SEQ ID NO: 18, T or S at an aa position corresponding to aa124 of SEQ ID NO: 18, T or S at an aa position corresponding to aa241 of SEQ ID NO: 18, T or S at an aa position corresponding to aa376 of SEQ ID NO: 18, N at an aa position corresponding to aa611 of SEQ ID NO: 18, T or S at an aa position corresponding to aa644 of SEQ ID NO: 18, N at an aa position corresponding to aa825 of SEQ ID NO: 18, and N at an aa position corresponding to aa826 of SEQ ID NO: 18;
T or S at an aa position corresponding to aa85 of SEQ ID NO: 26 or 28, T or S at an aa position corresponding to aa119 of SEQ ID NO: 26 or 28, T or S at an aa position corresponding to aa125 of SEQ ID NO: 26 or 28, T or S at an aa position corresponding to aa239 of SEQ ID NO: 26 or 28, T or S at an aa position corresponding to aa374 of SEQ ID NO: 26 or 28, N at an aa position corresponding to aa609 of SEQ ID NO: 26 or 28, N at an aa position corresponding to aa610 of SEQ ID NO: 26 or 28, T or S at an aa position corresponding to aa642 of SEQ ID NO: 26 or 28, and N at an aa position corresponding to aa823 of SEQ ID NO: 26 or 28; or
T or S at an aa position corresponding to aa64 of SEQ ID NO: 30, T or S at an aa position corresponding to aa98 of SEQ ID NO: 30, T or S at an aa position corresponding to aa104 of SEQ ID NO: 30, T or S at an aa position corresponding to aa218 of SEQ ID NO: 30, T or S at an aa position corresponding to aa353 of SEQ ID NO: 30, N at an aa position corresponding to aa588 of SEQ ID NO: 30, N at an aa position corresponding to aa589 of SEQ ID NO: 30, T or S at an aa position corresponding to aa621 of SEQ ID NO: 30, and N at an aa position corresponding to aa802 of SEQ ID NO: 30.

44. The recombinant Ube3a polypeptide or the biological equivalent thereof of claim 35, comprising one or more non-naturally occurring glycosylation sites.

45. The recombinant Ube3a protein, polypeptide or the biological equivalent thereof of claim 35, comprising an amino acid sequence selected from the following:
aa21 to aa 872 of SEQ ID NO: 14, aa21 to aa 895 of SEQ ID NO: 16, aa21 to aa 892 of SEQ ID NO: 18, aa21 to aa 890 of SEQ ID NO: 26, aa21 to aa 890 of SEQ ID NO: 28, aa21 to aa 869 of SEQ ID NO: 30, or a sequence having at least about 90%, or at least about 95%, or at least about 99% identity to each thereof.

46.-48. (canceled)

49. The recombinant Ube3a polypeptide or the biological equivalent thereof of claim 35, wherein the signal peptide comprises an amino acid sequence of aa1 to aa20 of SEQ ID NO: 14.

50. The recombinant Ube3a polypeptide or the biological equivalent thereof of claim 35, wherein the Ube3a polypeptide or a biological equivalent thereof comprises an amino acid sequence selected from any one of SEQ ID NOs: 14, 16, 18, 26, 28 and 30, or a sequence having at least about 90%, or at least about 95%, or at least about 99% identity to each thereof.

51. The recombinant Ube3a polypeptide or the biological equivalent thereof of claim 35, further comprising a self-cleaving peptide and a detectable or selection polypeptide.

52. An isolated or engineered cell comprising one or more of the following: a polynucleotide of claim 1.

53.-58. (canceled)

59. An isolated cell population comprising a cell of claim 52 or a progeny thereof.

60.-65. (canceled)

66. A method to treat, prevent, halt or reverse Angelman syndrome in a subject carrying a defective Ube3a allele, comprising locally or systemically administering to the subject a polynucleotide of claim 1.

67. A method to express a Ube3a protein or polypeptide or a biological equivalent thereof in a subject, comprising administering to the subject a polynucleotide of claim 1, thereby expressing Ube3a in the subject.

68. The method of claim 67, wherein the subject carries a defective Ube3a gene.

69. (canceled)

70. The method of claim 66, wherein the subject is symptomatic or asymptomatic for Angelman syndrome.

71.-75. (canceled)

76. An isolated or engineered cell comprising a polynucleotide of claim 1.

77.-79. (canceled)

80. A viral packaging system comprising: (a) the vector of claim 27; (b) one or more packaging plasmids; and (c) an envelope plasmid.

81.-82. (canceled)

83. A method for producing a viral particle, comprising transducing a packaging cell line with the system of claim 80 under conditions suitable to package the viral particle.

84. (canceled)

85. A method to express a secreted Ube3a polypeptide or the biological equivalent thereof comprising growing a cell of claim 52 under conditions that allow for the expression of the recombinant Ube3a polypeptide or a biological equivalent thereof.

86. (canceled)

87. A composition comprising a carrier and a polynucleotide of claim 1.

88. (canceled)

89. A kit comprising a polynucleotide of claim 1.
